(19) United States
(12) Patent Application Publication
(10) Pub. No.: US 2014/0127749 A1
(43) Pub. Date: May 8, 2014

(54) METHODS OF PROTEIN PRODUCTION AND COMPOSITIONS THEREOF

(71) Applicants: Hugh S. Mason, Phoenix, AZ (US); Fan Hong, Dallas, TX (US); George Bjorklund, Chandler, AZ (US)

(72) Inventors: Hugh S. Mason, Phoenix, AZ (US); Fan Hong, Dallas, TX (US); George Bjorklund, Chandler, AZ (US)

(73) Assignee: ARIZONA BORAD OF REGENTS, A BODY CORPORATE OF THE STATE OF ARIZONA ACTING FOR AND ON BEHALF OF ARIZO, Scottsdale, AZ (US)

(21) Appl. No.: 14/113,201

(22) PCT Filed: Apr. 23, 2012
(86) PCT No.: PCT/US12/34707

§ 371 Date: Oct. 21, 2013

Related U.S. Application Data
(60) Provisional application No. 61/478,019, filed on Apr. 21, 2011.

(51) Int. Cl. C07K 14/005

(52) U.S. Cl. CPC C07K 14/005
USPC 435/69.1; 435/419; 435/414; 435/411; 435/320.1; 435/252.3

(57) ABSTRACT

The invention provides methods for making a target protein in a plant cell, and compositions thereof, wherein the target protein is a recombinant viral glycoprotein.

### RELATED APPLICATION

This application claims priority under 35 U.S.C. 119(e) to provisional application U.S. Ser. No. 61/478,019, filed Apr. 21, 2011, which application is incorporated hereby by reference.

### GOVERNMENT FUNDING

The invention described herein was made with government support under Grant Number NIH-U19-A1-066332 awarded by the National Institutes of Health. The United States Government has certain rights in the invention.

### BACKGROUND OF THE INVENTION

Vaccination is currently regarded as the most effective way of preventing infectious diseases. Therefore, efforts are being made to develop new effective and safe vaccines against a variety of infectious diseases. A recombinant viral protein vaccine is one type of vaccine that uses the protein components of a virus, which are immunogenic but not infectious, to induce immune responses in a host. This type of vaccine is considered safer than live or killed virus vaccines because it lacks the viral nucleic acids which are responsible for viral replication. However, large recombinant proteins often fold poorly in the Endoplasmic Reticulum (ER), reducing the yield of the native form of the protein, thereby impeding vaccine development and increasing vaccine production costs. Therefore, new methods are needed to enhance the ER's ability to fold newly synthesized or misfolded polypeptides into native proteins.

### SUMMARY OF THE INVENTION

Accordingly, as described herein are methods to enhance the ER's ability to fold newly synthesized or misfolded polypeptides into native proteins, and compositions thereof.

Certain embodiments of the invention provide a plant cell comprising a first vector comprising two expression cassettes, a first expression cassette comprising a nucleic acid encoding a target protein, wherein the target protein is a recombinant viral glycoprotein, and a second expression cassette comprising a nucleic acid encoding a plant endoplasmic reticulum (ER) protein.

Certain embodiments of the invention provide a plant cell comprising a first vector comprising an expression cassette comprising a nucleic acid encoding a target protein, wherein the target protein is a recombinant viral glycoprotein, and a second vector comprising an expression cassette comprising a nucleic acid encoding a plant endoplasmic reticulum (ER) protein.

Certain embodiments of the invention provide a method of making a target protein comprising isolating the target protein from a plant cell that has been inoculated with a first stain of Agrobacterium (e.g., Agrobacterium tumefaciens) comprising a first vector comprising two expression cassettes wherein, a first expression cassette comprises a nucleic acid encoding a target protein and a second expression cassette comprises a nucleic acid encoding a plant endoplasmic reticulum (ER) protein, and wherein the target protein is a recombinant viral glycoprotein.

Certain embodiments of the invention provide a method of making a target protein comprising isolating the target protein from a plant cell that has been inoculated with a first strain of Agrobacterium (e.g., Agrobacterium tumefaciens) comprising a first vector comprising an expression cassette comprising a nucleic acid encoding a target protein and that has been inoculated with a second strain of Agrobacterium (e.g., Agrobacterium tumefaciens) comprising a second vector comprising an expression cassette comprising a nucleic acid encoding a plant endoplasmic reticulum (ER) protein, wherein the target protein is a recombinant viral glycoprotein.

Certain embodiments of the invention provide a vector comprising two expression cassettes, a first expression cassette comprising a nucleic acid encoding a target protein, wherein the target protein is a recombinant viral glycoprotein, and a second expression cassette comprising a nucleic acid encoding a plant endoplasmic reticulum (ER) protein.

Certain embodiments of the invention provide a strain of Agrobacterium (e.g. Agrobacterium tumefaciens) comprising a vector as described herein.

Certain embodiments of the invention provide a composition comprising a target protein as described herein and a physiologically-acceptable, non-toxic vehicle.

Certain embodiments of the invention provide a method of eliciting an immune response in an animal comprising introducing into the animal the composition as described herein.

Certain embodiments of the invention provide a method of generating antibodies specific for an antigen in an animal, comprising introducing into the animal a composition as described herein.

Certain embodiments of the invention provide a method of treating or preventing a viral infection in a patient in need of such treatment, comprising administering to the patient a composition as described herein.

Certain embodiments of the invention provide a composition as described in herein for use in medical treatment.

Certain embodiments of the invention provide a use of a composition as described herein to prepare a medicament useful for treating or preventing a viral infection in an animal. Certain embodiments of the invention provide a composition as described herein for use in treating or preventing a viral infection.

### BRIEF DESCRIPTION OF THE FIGURES

FIG. 1. A. Schematic representation of the T-DNA region of the vectors used in Example 1. 35S/TEV5′: CaMV 35S promoter with tobacco etch virus 5′UTR; VSP3′: soybean vspB gene 3′ element; 35S/TMV5′: CaMV 35S promoter with tobacco mosaic virus 5′UTR; Ext 3′: tobacco extensin 3′ UTR; p NOS: nopaline synthase promoter; NOS 3′: nopaline synthase 3′ UTR; NPT II: expression cassette encoding nptII gene for kanamycin resistance; LIR: long intergenic region of bean yellow dwarf geminivirus (BeYDV) genome; SIR: short intergenic region of BeYDV genome; C1/C2: BeYDV ORFs C1 and C2, encoding Rep and RepA for viral replication; LB and RB: the left and right border of the T-DNA region. B. HCV gpE2 plant-optimized coding sequence in pBYRsE2TR. C. Map of psNV120 and nucleotide sequence thereof. The coding sequence of AtbZIP60. E. The coding sequence of AtbZIP60ΔC.

FIG. 2. Phenotype observation of a leaf spot expressing soluble HCV E2 at 4, 6, 8, and 10 days post infiltration. The upper leaf spot was infiltrated with an empty viral vector to be set as a negative control for determination of the phytotoxic effect.

FIG. 3. Western blot analysis of soluble forms of HCV E2 production in N. benthamiana leaves. (A) Analysis of denatured sE2 using a mouse linear E2 antibody. Lane 1 to 4: denatured total soluble proteins of crude leaf extracts from samples harvested at 4, 8, 10 and 12 dpi. Lane 5: purified E2-IgG heavy chain fusion protein, denatured (positive control). (B) Analysis of conformational sE2 using a mouse conformational E2 antibody. Lane 1 to 4: total soluble proteins of crude leaf extracts from samples harvested at 4, 8, 10 and 12 dpi. Lane 5: purified E2-IgG heavy chain fusion protein (positive control).

FIG. 4. RT-PCR showing the abundance of AtCNX and AtCRT transcripts in samples infiltrated with psAtCRT-ext or psAtCNX-ext at 2 dpi. Lane 1: leaf infiltrated with psAtCNX-ext; Lane 2: pBYR-AtCNX plasmid, positive control; Lane 3: wild type leaf; Lane 4: negative control. Lane 5: leaf infiltrated with psAtCRT-ext; Lane 6: pBYR-AtCRT plasmid, positive control; Lane 7: wild type leaf; Lane 8: negative control. AtCNX was amplified using primers AtCNX-Xba-F and AtCNX-Kpn-R; AtCRT was amplified using primers AtCRT-Xba-F and AtCRT-Kpn-R.

FIG. 5. Phenotype observation of leaf spots at 3, 4 and 6 days post infiltration expressing soluble HCV E2 alone (upper left), soluble HCV E2 and calreticulin (upper right), and negative control (bottom). The upper left spot was co-infiltrated with pBYRsE2-711H and pPS1 at 1:1 ratio, the upper right spot was co-infiltrated with pBYRsE2-711H and psAtCRT-ext at 1:1 ratio, and the bottom spot was infiltrated with pPS1. The total OD₆₀₀ for infiltration was 0.2.

FIG. 6. Western blot analysis of soluble form of HCV E2 production with or without co-expression of Arabidopsis calreticulin in N. benthamiana leaves. (A) Reducing Western blots comparing denatured sE2 levels in sE2/pPS1 samples and sE2/calreticulin samples from same leaves harvested at 4 dpi and 8 dpi, using mouse anti-linear E2 antibodies. Protein samples were denatured in SDS sample buffer containing 150 Mm DTT and boiling. (B) Non-reducing Western blots comparing correct folded sE2 levels in sE2/pPS1 samples and sE2/calreticulin samples from same leaves harvested at 4 dpi and 8 dpi, using mouse anti-conformational E2 antibodies. Protein samples were mixed with SDS sample buffer without DTT and were not boiled. Lane 1 and 2: soluble protein extracts from two different sE2/pPS1 samples. Lane 3 and 4: soluble protein extracts from two different sE2/calreticulin samples. Lane 5: purified E2-IgG heavy chain fusion protein (positive control).

FIG. 7. Phenotype observation of leaf spots expressing membrane bound HCV E2 alone (upper left), membrane bound HCV E2 and calnexin (upper right), empty vector pPS1 (bottom left), and calnexin (bottom right) at 5 dpi. The upper left spot was co-infiltrated with pBYRsE2TR and pPS1, the upper right spot was co-infiltrated with pBYRsE2TR and psAtCNX-ext, the bottom left spot was infiltrated with pPS1, the bottom right spot was infiltrated with psAtCNX-ext. The total OD₆₀₀ for infiltration was 0.2, therefore the OD₆₀₀ of each construct is 0.1.

FIG. 8. Western blot analysis of the membrane bound HCV E2 production with or without co-expression of Arabidopsis calnexin in N. benthamiana leaves. (A) A reducing Western blot comparing denatured mE2 levels in mE2/pPS1 samples and mE2/calnexin samples from same leaves harvested at 5 dpi, using mouse anti-linear E2 antibodies. Protein samples were denatured in SDS sample buffer containing 150 Mm DTT and boiled. (B) A non-reducing Western blot comparing correct folded sE2 levels in mE2/pPS1 samples and mE2/calnexin samples from same leaves harvested at 5 dpi, using mouse anti-conformational E2 antibodies. Protein samples were mixed with SDS sample buffer without DTT and were not boiled. Lane 1: supernatant of leaf crude extract from mE2/pPS1 sample. Lane 2: pellet of leaf crude extract from mE2/pPS1 sample. Lane 3: supernatant of leaf crude extract from sE2/calnexin sample. Lane 4: pellet of leaf crude extract from sE2/calnexin sample. Lane 5: purified E2-IgG heavy chain fusion protein (positive control).

FIG. 9. Phenotype observation of leaf spots expressing membrane bound HCV E2, calreticulin and calnexin (upper left), membrane bound HCV E2 alone (upper right), calreticulin and calnexin (bottom left), and empty vector pPS1 (bottom right) at 5 dpi. The upper left spot was co-infiltrated with pBYRsE2TR, psAtCRT-ext and psAtCNX-ext, the upper right spot was co-infiltrated with pBYRsE2TR and pPS1, the bottom left spot was infiltrated with psAtCRT-ext and psAtCNX-ext, and the bottom right spot was infiltrated with pPS1. The total OD₆₀₀ for infiltration was 0.3.

FIG. 10. Western blot analysis of membrane bound HCV E2 production with or without co-expression of Arabidopsis calnexin and calreticulin in N. benthamiana leaves. (A) A reducing western blot comparing denatured mE2 levels in mE2/pPS1 samples and mE2/calnexin/calreticulin samples from same leaves, using mouse anti-linear E2 antibodies. Protein samples were denatured in SDS sample buffer containing 150 Mm DTT and boiled. (B) A non-reducing Western blot comparing correct folded sE2 levels in mE2/pPS1 samples and mE2/calnexin/calreticulin samples from same leaves, using mouse anti-conformational E2 antibodies. Protein samples were not denatured and boiled. Lane 1: supernatant of leaf crude extract of mE2/pPS1 sample. Lane 2: pellet of leaf crude extract of mE2/pPS1 sample. Lane 3: supernatant of leaf crude extract of sE2/calnexin sample. Lane 4: pellet of leaf crude extract of sE2/calnexin sample. Lane 5: purified E2-IgG heavy chain fusion protein (positive control).

FIG. 11. RT-PCR products amplified from samples expressing (A) NbbZIP60, (B) NbbZIP60ΔC, (C) AtbZIP60 or AtbZIP60ΔC. Constitutively expressed N. benthamiana EF1α was used as the internal control and was amplified using primers EF1α-F and EF1α-R. WT stands for wild type sample. (−) stands for negative control. In (A), NbbZIP60 was amplified using primers NbbZIP60-Nco-F and NbbZIP60-Sac-R. In (B), NbbZIP60ΔC was amplified using primers NbbZIP60-Nco-F and NbbZIP60-S212. In (C), AtbZIP60 was amplified using primers pUni51-F and AtbZIP60-Kpn-R; AtbZIP60ΔC was amplified using primers pUni51-F and AtbZIP60-S216-K.

FIG. 12. Phenotype observation of a leaf expressing soluble HCV E2 and NbbZIP60 (spot 1), soluble HCV E2 alone (spot 2), HCV E2 and NbbZIP60ΔC (spot 3), and HCV E2 and AtbZIP60ΔC (spot 4) at 4, 6 and 8 days post infiltration. Another leaf expressing HCV E2 alone (spot 5) and HCV E2 and AtbZIP60 (spot 6) at 8 dpi was also shown on the right. Spot 1 was co-infiltrated with pBYRsE2-711H and NosNbZ60, spot 2 and 5 were co-infiltrated with pBYRsE2-711H and pPS1, spot 3 was co-infiltrated with pBYRsE2-711H and NosNbZS212, spot 4 was co-infiltrated with pBYRsE2-711H and psAtbZIP60 and spot 6 was co-infiltrated with pBYRsE2-711H and psAtbZIP60-S216. The total OD₆₀₀ for infiltration was 0.2, so that the OD₆₀₀ of each construct is 0.1.

FIG. 13. Western blot analysis of expression of soluble form of HCV E2 with bZIP60 or bZIP60ΔC treatments at 4 and 8 dpi. (A) Reducing Western blots comparing denatured sE2 levels in sE2/pPS1 samples and sE2/treatment samples from same leaves harvested at 4 dpi and 8 dpi, using mouse anti-linear E2 antibodies. Protein samples were denatured in SDS sample buffer containing 150 mM DTT and boiled. (B) Non-reducing Western blot comparing correct folded sE2 levels in sE2/pPS1 samples and sE2/treatment samples from same leaves harvested at 4 dpi and 8 dpi, using mouse anti-conformational E2 antibodies. Protein samples were mixed with SDS sample buffer without DTT and were not boiled. Lane 1: sE2/NbbZIP60 sample. Lane 2: sE2/AtbZIP60 sample. Lane 3: sE2/AtbZIP60ΔC sample. Lane 4: sE2/NbbZIP60ΔC sample. Lane 5: sE2/pPS1 sample. Lane 6: purified E2-IgG heavy chain fusion protein (positive control).

FIG. 14. Western blot analysis of expression of soluble form of HCV E2 with NbbZIP60 or AtbZIP60ΔC treatments at 4 and 8 dpi. (A) Reducing Western blots comparing denatured sE2 levels in sE2/pPS1 samples and sE2/treatment samples from same leaves harvested at 4 dpi and 8 dpi, using mouse anti-linear E2 antibodies. Protein samples were denatured in SDS sample buffer containing 150 Mm DTT and boiled. (B) Non-reducing Western blot comparing correctly folded sE2 levels in sE2/pPS1 samples and sE2/treatment samples from same leaves harvested at 4 dpi and 8 dpi, using mouse anti-conformational E2 antibodies. Protein samples were mixed with SDS sample buffer without DTT and were not boiled. Lane 1 and 2: two different sE2/pPS1 samples. Lane 3 and 4: two different sE2/NbbZIP60 samples. Lane 5 and 6: two different sE2/AtbZIP60ΔC samples. Lane 7: purified E2-IgG heavy chain fusion protein (positive control).

FIG. 15. RT-PCR showing the abundance of Blp1, Blp2, Blp4 and Blp8 transcripts from samples with indicated treatment harvested at 2 dpi. Constitutively expressed N. benthamiana EF1α was used as the internal control and was amplified using primers EF1α-F and EF1α-R. Blp1 was amplified using primers NtBlp1-F and NtBlp1-R; Blp2 was amplified using primers NtBlp2-F and NtBlp2-R; Blp4 was amplified using primers NtBlp4-F and NtBlp4-R; Blp8 was amplified using primers NtBlp8-F and NtBlp8-R.

FIG. 16. Coding sequence of Nicotiana tabacum bZip60 gene.

FIG. 17. Representations of the T-DNA vectors used in the analysis of the Ebola GP1 protein expression with and without calreticulin co-expression. LB, T-DNA left border; RB, T-DNA right border; LIR, geminiviral long intergenic region; SIR, geminiviral short intergenic region; P19, expression cassette for gene silencing suppressor p19 driven by nopaline synthase promoter; expression cassette for GP1/H2, Ebola GP1-heavy chain fusion driven by CaMV 35S promoter; C1/R1, geminiviral Rep/RepA gene; Calreticulin, expression cassette for calreticulin driven by nopaline synthase promoter. The geminiviral replicon lies between the 2 LIR elements.

FIG. 18. Calreticulin enhancement of Ebola GP1-H2 fusion protein expression. Two different leaves were agroinoculated with T-DNA vectors pBYR-P-gp1dH2-C (CRT+p19) or pBYR-P-gp1dH2 (p19, no CRT) shown in FIG. 17. Both vectors were inoculated on either side of the same leaves in order to minimize experimental variance. Soluble (S) and insoluble pellet (P) fractions were electrophoresed in a 4-12% SDS-PAGE gradient gel under non-reducing conditions, then blotted to a PVDF membrane. GP1 proteins were probed using conformation-dependent anti-GP1 mouse monoclonal antibody 13C6, and detected using a goat anti-mouse IgG-HRP conjugate.

FIG. 19. A. Nucleotide sequence of N. benthamiana bZIP60 cDNA (nt 1-3, start codon; nt 898-900, stop codon). B. N. benthamiana bZIP60 spliced cDNA (nt 1-3, start codon; nt 769-771, stop codon).

FIG. 20. Map of pZIP60sfv.

FIG. 21. Map of pNTNbbZ60sf.

FIG. 22. Map of pZIP60sf120.

FIG. 23. Map of pBYRbZ60-gpldH2.

FIG. 24. A. Gene sequence for A/California/07/2009(H1N1) hemagglutinin gene (nt 1-99=signal peptide; nt 100-1746=HA; nt 1747-1749=stop codon). B. Gene sequence for C-terminal truncated A/California/07/2009(H1N1) hemagglutinin gene (nt 1-99=signal peptide; nt 100-1626=HA ectodomain; nt 1627-1629=stop codon).

FIG. 25. Map of pBYR2efb-HA.

FIG. 26. Map of pBYR2fc-HA.

FIG. 27. Map of pBYR2fb-cH106.

FIG. 28. Map of pBYR2fper-cH106.

FIG. 29. Map of pBYR2fd-cH106.

### DETAILED DESCRIPTION

Newly synthesized polypeptides must undergo folding and assembly in the endoplasmic reticulum (ER) to obtain a unique native structure. This process is usually coordinated with post-translational modifications such as N-linked glycosylation and disulfide bond formation.

Nascent polypeptide chains may misfold and aggregate in the ER, especially those proteins whose mature structures are complex (e.g., viral glycoproteins). Incorrect folding may result in the inhibition of interactions with other molecules and/or disrupt protein function. However, the ER contains molecular chaperones which are designed to facilitate protein folding by increasing the efficiency of the folding process. These chaperones transiently bind to nascent and incompletely folded polypeptide chains and are thought to have functions in preventing intramolecular or intermolecular aggregation, suppressing pre-matured protein degradation, and facilitating ER folding factors to catalyze protein folding.

Accordingly, certain embodiments of the present invention provide a plant cell comprising a first vector comprising two expression cassettes, a first expression cassette comprising a nucleic acid encoding a target protein, wherein the target protein is a recombinant viral glycoprotein, and a second expression cassette comprising a nucleic acid encoding a plant endoplasmic reticulum (ER) protein.

In certain embodiments, the vector further comprises a third expression cassette comprising a nucleic acid encoding a plant endoplasmic reticulum protein.

In certain embodiments, the nucleic acid in the second expression cassette encodes an ER protein that is different than the ER protein encoded by the nucleic acid in the third expression cassette.

In certain embodiments, the cell further comprises a second vector comprising an expression cassette comprising a nucleic acid encoding a plant endoplasmic reticulum protein.

In certain embodiments, the ER protein of the first vector is different than the ER protein of the second vector.

Certain embodiments of the invention provide a plant cell comprising a first vector comprising an expression cassette comprising a nucleic acid encoding a target protein, wherein the target protein is a recombinant viral glycoprotein, and a second vector comprising an expression cassette comprising a nucleic acid encoding a plant endoplasmic reticulum (ER) protein.

In certain embodiments, the first vector or the second vector comprises a second expression cassette comprising a nucleic acid encoding a plant ER protein.

Certain embodiments further comprise a third vector comprising an expression cassette comprising a nucleic acid encoding a plant ER protein.

In certain embodiments, the ER protein of the second vector is different than the ER protein of the second expression cassette or the ER protein of the third vector.

In certain embodiments, the ER protein is an Arabidopsis thaliana, Nicotiana tabacum, Solanum lycopersicum or Nicotiana benthamiana protein.

As used herein, an ER protein may be a protein that is localized in the ER. In certain embodiments, the ER protein is localized only in the ER. In certain embodiments, the ER protein may serve a specialized function in the ER (e.g., promoting protein folding) or may function in an ER associated pathway (e.g., activation of gene expression, for example, in the unfolded protein response (UPR) pathway).

In certain embodiments, the ER protein is a molecular chaperone or a transcription factor.

In certain embodiments, the ER protein is a molecular chaperone.

In certain embodiments, the molecular chaperone is selected from a general chaperone, a lectin chaperone and a non-classical chaperone.

In certain embodiments, the molecular chaperone is a lectin chaperone.

In certain embodiments, the lectin chaperone is calnexin or calreticulin.

In certain embodiments, the lectin chaperone is calnexin.

In certain embodiments, the lectin chaperone is calreticulin.

In certain embodiments, the chaperone is a general chaperone.

In certain embodiments, the general chaperone is a Binding immunoglobulin protein (BiP).

In certain embodiments, the ER protein is a transcription factor.

In certain embodiments, the transcription factor induces expression of an unfolded protein response (UPR) gene.

In certain embodiments, the transcription factor induces expression of a binding immunoglobulin protein (BiP) gene.

In certain embodiments, the BiP gene is a luminal binding protein (Blp) gene (e.g., Blp1, Blp2, Blp4, or Blp8).

In certain embodiments, the transcription factor binds to a stress response element (ERSE) or UPR element in the promoter of the UPR gene.

In certain embodiments, the transcription factor is basic leucine zipper transcription factor 60 (bZIP60) or basic leucine zipper transcription factor 28 (bZIP28).

In certain embodiments, the transcription factor is bZIP60.

In Arabidopsis thaliana, heat stress induces the cytoplasmic splicing of bZIP60 mRNA to create a coding sequence that includes a C-terminal nuclear targeting signal (Deng et al., (2011) Proc Natl Acad Sci USA. 2011 Apr. 26; 108(17):7247-52). Transport of the protein product of the spliced bZIP60 mRNA into the nucleus results in upregulation of genes related to ER stress, including chaperones.

Accordingly, in certain embodiments, bZIP60 comprises a nuclear targeting signal (e.g., C-terminal).

In certain embodiments, the transcription factor is bZIP28.

In certain embodiments, the glycoprotein is selected from a HCV protein, an Ebola virus protein and an influenza protein.

In certain embodiments, the glycoprotein is an HCV protein.

In certain embodiments, the HCV protein is HCV envelope protein 2 (E2).

In certain embodiments, the glycoprotein is an Ebola virus protein.

In certain embodiments, the Ebola virus protein comprises an Ebola virus glycoprotein (GP1).

In certain embodiments, the GP1 protein is operably linked to the heavy chain of anti-GP1 monoclonal antibody 6D8.

In certain embodiments, the glycoprotein is an influenza protein.

In certain embodiments, the influenza protein is Influenza virus hemagglutinin (HA).

In certain embodiments, the vector is a geminiviral vector or a non-replicating binary vector.

In certain embodiments, the cell is an Arabidopsis thaliana, Nicotiana tabacum, Solanum lycopersicum, or Nicotiana benthamiana cell.

Certain embodiments provide a method of making a target protein comprising isolating the target protein from a plant cell that has been inoculated with a first stain of Agrobacterium (e.g., Agrobacterium tumefaciens) comprising a first vector comprising two expression cassettes wherein, a first expression cassette comprises a nucleic acid encoding a target protein and a second expression cassette comprises a nucleic acid encoding a plant endoplasmic reticulum (ER) protein, and wherein the target protein is a recombinant viral glycoprotein.

In certain embodiments, the vector further comprises a third expression cassette comprising a nucleic acid encoding a plant endoplasmic reticulum protein.

In certain embodiments, the nucleic acid in the second expression cassette encodes an ER protein that is different than the ER protein encoded by the nucleic acid in the third expression cassette.

In certain embodiments, the cell has further been inoculated with a second strain of Agrobacterium (e.g., Agrobacterium tumefaciens) comprising a second vector comprising an expression cassette comprising a nucleic acid encoding a plant endoplasmic reticulum protein.

In certain embodiments, the ER protein of the first vector is different than the ER protein of the second vector.

Certain embodiments of the invention provide a method of making a target protein comprising isolating the target protein from a plant cell that has been inoculated with a first strain of Agrobacterium (e.g., Agrobacterium tumefaciens) comprising a first vector comprising an expression cassette comprising a nucleic acid encoding a target protein and that has been inoculated with a second strain of Agrobacterium (e.g., Agrobacterium tumefaciens) comprising a second vector comprising an expression cassette comprising a nucleic acid encoding a plant endoplasmic reticulum (ER) protein, wherein the target protein is a recombinant viral glycoprotein.

In certain embodiments, the first vector or the second vector comprises a second expression cassette comprising a nucleic acid encoding a plant ER protein.

In certain embodiments, the cell has further been inoculated with a third strain of Agrobacterium (e.g., Agrobacterium tumefaciens) comprising third vector comprising an expression cassette comprising a nucleic acid encoding a plant ER protein.

In certain embodiments, the ER protein of the second vector is different than the ER protein of the second expression cassette or the ER protein of the third vector.

In certain embodiments, the target protein is isolated, e.g, 1, 2, 3, 4, 5, 6, 7, 8, 9, 10, 11, 12, 13, 14, 15 days post-infiltration (dpi). In certain embodiments, the target protein is isolated 3 dpi. In certain embodiments, the target protein is isolated 4 dpi. In certain embodiments, the target protein is isolated 5 dpi. In certain embodiments, the target protein is isolated 6 dpi. In certain embodiments, the target protein is isolated 8 dpi. In certain embodiments, the target protein is isolated 10 dpi. In certain embodiments, the target protein is isolated 12 dpi.

In certain embodiments, an increased amount of properly folded target protein is isolated from the plant cell as compared to an amount of properly folded glycoprotein isolated from a plant cell that was not inoculated with a strain of Agrobacterium (e.g., Agrobacterium tumefaciens) comprising a vector comprising an expression cassette comprising a nucleic acid encoding a plant ER protein.

In certain embodiments, the amount of properly folded target protein isolated from the plant cell is increased by, e.g., about 10%, 20%, 30%, 40%, 50%, 60%, 70%, 80%, 90%, 100%, 110%, 120%, 130%, 140%, 150%, 175%, 200%, 250%, 300%, 350%, 400%, 450%, 500%, 600%, 700%, 800%, 900%, 1,000%, 1,100%, 1,200%, 1,300%, 1,400%, 1,500%, 1,600%, 1,700%, 1,800%, 1,900% or 2,000% over an amount of properly folded glycoprotein isolated from a plant cell that was not inoculated with a strain of Agrobacterium (e.g., Agrobacterium tumefaciens) comprising a vector comprising an expression cassette comprising a nucleic acid encoding a plant ER protein.

In certain embodiments, the Agrobacterium is Agrobacterium tumefaciens.

In certain embodiments the Agrobacterium tumefaciens strain is GV3101.

In certain embodiments, the ER protein is an Arabidopsis thaliana, Nicotiana tabacum, Solanum lycopersicum or Nicotiana benthamiana protein.

In certain embodiments, the ER protein is a molecular chaperone or a transcription factor.

In certain embodiments, the ER protein is a molecular chaperone.

In certain embodiments, the molecular chaperone is selected from a general chaperone, a lectin chaperone and a non-classical chaperone.

In certain embodiments, the molecular chaperone is a lectin chaperone.

In certain embodiments, the lectin chaperone is calnexin or calreticulin.

In certain embodiments, the lectin chaperone is calnexin.

In certain embodiments, the lectin chaperone is calreticulin.

In certain embodiments, the chaperone is a general chaperone.

In certain embodiments, the general chaperone is a Binding immunoglobulin protein (BiP).

In certain embodiments, the ER protein is a transcription factor.

In certain embodiments, the transcription factor induces expression of an unfolded protein response (UPR) gene.

In certain embodiments, the transcription factor induces expression of a binding immunoglobulin protein (BiP) gene.

In certain embodiments, the BiP gene is a luminal binding protein (Blp) gene (e.g., Blp1, Blp2, Blp4, or Blp8).

In certain embodiments, the transcription factor binds to a stress response element (ERSE) or UPR element in the promoter of the UPR gene.

In certain embodiments, the transcription factor is basic leucine zipper transcription factor 60 (bZIP60) or basic leucine zipper transcription factor 28 (bZIP28).

In certain embodiments, the transcription factor is bZIP60.

In certain embodiments, bZIP60 comprises a nuclear targeting signal (e.g., C-terminal).

In certain embodiments, the transcription factor is bZIP28.

In certain embodiments, the glycoprotein is selected from a HCV protein, an Ebola virus protein and an influenza protein.

In certain embodiments, the glycoprotein is an HCV protein.

In certain embodiments, the HCV protein is HCV envelope protein 2 (E2).

In certain embodiments, more of the E2 protein binds to a conformation-sensitive anti-E2 antibody as compared to an E2 protein isolated from a plant cell that was not inoculated with a strain of Agrobacterium (e.g., Agrobacterium tumefaciens) comprising a vector comprising an expression cassette comprising a nucleic acid encoding a plant ER protein.

In certain embodiments, the conformation-sensitive anti-E2 antibody is mouse monoclonal antibody 5E5H7 (Novartis).

In certain embodiments, more of the E2 protein binds to cell receptor CD81 as compared to an E2 protein isolated from a plant cell that was not inoculated with a strain of Agrobacterium (e.g., Agrobacterium tumefaciens) comprising a vector comprising an expression cassette comprising a nucleic acid encoding a plant ER protein.

In certain embodiments, the glycoprotein is an Ebola virus protein.

In certain embodiments, the Ebola virus protein comprises an Ebola virus glycoprotein (GP1).

In certain embodiments, the GP1 protein is operably linked to the heavy chain of anti-GP1 monoclonal antibody 6D8.

In certain embodiments, more of the GP1 protein binds to a conformation-sensitive anti-GP1 antibody as compared to a GP1 protein isolated from a plant cell that was not inoculated with a strain of Agrobacterium (e.g., Agrobacterium tumefaciens) comprising a vector comprising an expression cassette comprising a nucleic acid encoding a plant ER protein.

In certain embodiments, the conformation-sensitive anti-GP1 antibody is conformation-sensitive anti-GP1 mouse monoclonal antibody 13C6.

In certain embodiments, the glycoprotein is an influenza protein.

In certain embodiments, the influenza protein is Influenza virus hemagglutinin (HA).

In certain embodiments, more of the HA protein binds to a conformation-sensitive anti-HA antibody as compared to a HA protein isolated from a plant cell that was not inoculated with a strain of Agrobacterium (e.g., Agrobacterium tumefaciens) comprising a vector comprising an expression cassette comprising a nucleic acid encoding a plant ER protein.

In certain embodiments, the conformation-sensitive anti-HA antibody is SEK001 (Sinobiologicals).

In certain embodiments, the vector is a geminiviral vector or a non-replicating binary vector.

In certain embodiments, the cell is an Arabidopsis thaliana, Nicotiana tabacum, Solanum lycopersicum, or Nicotiana benthamiana cell.

Certain embodiments provide a vector comprising two expression cassettes, a first expression cassette comprising a nucleic acid encoding a target protein, wherein the target protein is a recombinant viral glycoprotein, and a second expression cassette comprising a nucleic acid encoding a plant endoplasmic reticulum (ER) protein.

In certain embodiments, the ER protein is a molecular chaperone or a transcription factor.

In certain embodiments, the ER protein is a molecular chaperone.

In certain embodiments, the molecular chaperone is selected from a general chaperone, a lectin chaperone and a non-classical chaperone.

In certain embodiments, the molecular chaperone is a lectin chaperone.

In certain embodiments, the lectin chaperone is calnexin or calreticulin.

In certain embodiments, the lectin chaperone is calnexin.

In certain embodiments, the lectin chaperone is calreticulin.

In certain embodiments, the chaperone is a general chaperone.

In certain embodiments, the general chaperone is a Binding immunoglobulin protein (BiP).

In certain embodiments, the ER protein is a transcription factor.

In certain embodiments, the transcription factor induces expression of an unfolded protein response (UPR) gene.

In certain embodiments, the transcription factor induces expression of a binding immunoglobulin protein (BiP) gene.

In certain embodiments, the BiP gene is a luminal binding protein (Blp) gene (e.g., Blp1, Blp2, Blp4, or Blp8).

In certain embodiments, the transcription factor binds to a stress response element (ERSE) or UPR element in the promoter of the UPR gene.

In certain embodiments, the transcription factor is basic leucine zipper transcription factor 60 (bZIP60) or basic leucine zipper transcription factor 28 (bZIP28).

In certain embodiments, the transcription factor is bZIP60.

In certain embodiments, bZIP60 comprises a nuclear targeting signal (e.g., C-terminal).

In certain embodiments, the transcription factor is bZIP28.

In certain embodiments, the glycoprotein is selected from a HCV protein, an Ebola virus protein and an influenza protein.

In certain embodiments, the glycoprotein is an HCV protein.

In certain embodiments, the HCV protein is HCV envelope protein 2 (E2).

In certain embodiments, the glycoprotein is an Ebola virus protein.

In certain embodiments, the Ebola virus protein comprises an Ebola virus glycoprotein (GP1).

In certain embodiments, the GP1 protein is operably linked to the heavy chain of anti-GP1 monoclonal antibody 6D8.

In certain embodiments, the glycoprotein is an influenza protein.

In certain embodiments, the influenza protein is Influenza virus hemagglutinin (HA).

In certain embodiments, the vector is a geminiviral vector.

Certain embodiments of the invention provide a strain of Agrobacterium (e.g., Agrobacterium tumefaciens) comprising a vector as described herein.

Certain embodiments of the invention provide a composition comprising a target protein as described herein and a physiologically-acceptable, non-toxic vehicle.

In certain embodiments, the composition further comprises an adjuvant.

Certain embodiments of the invention provide a method of eliciting an immune response in an animal comprising introducing into the animal a composition as described herein.

Certain embodiments of the invention provide a method of generating antibodies specific for an antigen in an animal, comprising introducing into the animal a composition as described herein.

In certain embodiments, the animal is a human.

Certain embodiments of the invention provide a method of treating or preventing a viral infection in a patient in need of such treatment, comprising administering to the patient a composition as described herein.

Certain embodiments of the invention provide a composition as described herein for use in medical treatment.

Certain embodiments of the invention provide the use of a composition as described herein to prepare a medicament useful for treating or preventing a viral infection in an animal.

Certain embodiments of the invention provide a composition as described herein for use in treating or preventing a viral infection.

Certain embodiments of the invention provide a method for improving the recombinant production or yield of protein from a recombinant production in plant cells, which method comprises transiently overexpressing in a host plant cell a molecular chaperone of the endoplasmic reticulum, wherein the plant host cell expresses the recombinant protein.

In certain embodiments, said recombinant protein is a HCV protein.

In certain embodiments, said HCV protein is HCV envelope protein 2.

In certain embodiments, said molecular chaperone is calnexin, calreticulin or both calnexin and calreticulin.

In certain embodiments, said transient overexpression of said molecular chaperone of the endoplasmic reticulum in said host plant cell increases the efficiency of said recombinant protein folding as compared to the folding of said recombinant protein in the absence of transient overexpression of said molecular chaperone.

In certain embodiments, said transient overexpression of said molecular chaperone of the endoplasmic reticulum in said host plant cell reduces aggregation of said recombinant protein as compared to the aggregation of said recombinant protein in the absence of transient overexpression of said molecular chaperone.

In certain embodiments, the methods described herein further comprising overexpressing bZIP60 in said host plant cell.

In certain embodiments, said plant cell is a N. benthamiana cell.

Certain embodiments of the invention provide a method for improving the production of a recombinant HCV E2 protein, which method comprises transfecting a host N. benthamiana cell expressing the recombinant HCV protein with an expression construct wherein expression of said construct in said N. benthamiana that produces a transient expression of at least one or both of the molecular chaperones calnexin, calreticulin and said transient expression of the molecular chaperones leads to an improvement in the production or yield of recombinant HCV E2 protein from said N. benthamiana cell as compared to the production or yield of recombinant HCV E2 protein from said N. benthamiana cell that has not been transfected with such an expression construct.

### DEFINITIONS

The terms “protein,” “peptide” and “polypeptide” are used interchangeably herein.

The term “amino acid” includes the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also includes peptides with reduced peptide bonds, which will prevent proteolytic degradation of the peptide. Also, the term includes the amino acid analog α-amino-isobutyric acid. The term also includes natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

In certain embodiments, the peptides are modified by C-terminal amidation, head to tail cyclic peptides, or containing Cys residues for disulfide cyclization, siderophore modification, or N-terminal acetylation.

The term “peptide” describes a sequence of 7 to 50 amino acids or peptidyl residues. Preferably a peptide comprises 7 to 25, or 7 to 15 or 7 to 13 amino acids. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right.

By “variant” peptide is intended a peptide derived from the native peptide by deletion (so-called truncation) or addition of one or more amino acids to the N-terminal and/or C-terminal end of the native peptide; deletion or addition of one or more amino acids at one or more sites in the native peptide; or substitution of one or more amino acids at one or more sites in the native peptide. The peptides of the invention may be altered in various ways including amino acid substitutions, deletions, truncations, and insertions. Methods for such manipulations are generally known in the art. For example, amino acid sequence variants of the peptides can be prepared by mutations in the DNA. Methods for mutagenesis and nucleotide sequence alterations are well known in the art. The substitution may be a conserved substitution. A “conserved substitution” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall peptide retains its spatial conformation but has altered biological activity. For example, common conserved changes might be Asp to Glu, Asn or Gln; His to Lys, Arg or Phe; Asn to Gln, Asp or Glu and Ser to Cys, Thr or Gly Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cystine, tyrosine, asparagine and glutamine having uncharged polar side chains; aspartate and glutamate having acidic side chains; and lysine, arginine, and histidine having basic side chains.

As used herein, the term “glycoprotein” refers to a protein that contains oligosaccharide chains (glycans) covalently attached to polypeptide side-chains. The carbohydrate may be attached to the protein in a cotranslational or posttranslational modification.

Nucleic Acids of the Present Invention

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell or bacteriophage. For example, an “isolated” or “purified” nucleic acid molecule or protein, or biologically active portion is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals.

Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. A fragment of a nucleic acid sequence or protein may result from deletion (so-called truncation) of one or more nucleotides or amino acids from the N-terminal and/or C-terminal end of the native sequence/peptide; or modification (e.g., deletion, addition or substitution) of one or more nucleotides/amino acids at one or more sites in the native sequence or peptide. Generally, fragments and variants of the disclosed proteins or partial length proteins will retain native function or partial native function and fragments and variants of the disclosed nucleotide sequences will encode proteins or partial length proteins that retain native function or partial native function.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

The term “chimeric” refers to any gene or DNA that contains 1) DNA sequences, including regulatory and coding sequences that are not found together in nature or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, DNA that is either heterologous or homologous to the DNA of a particular cell to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, N.Y.: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, viral vectors, phage, or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

A “functional RNA” refers to an antisense RNA, ribozyme, or other RNA that is not translated.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency.

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. “Operably-linked” may also refer to the association of nucleic acids or proteins that are linked directly or indirectly (e.g, a nucleic acid encoding a fusion protein, or a fusion protein).

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a known mathematical algorithm. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (available on the world wide web at This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world-wide-web at ncbi.nlm.nih.gov. Alignment may also be performed manually by visual inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (TO for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tₘ) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl: Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

A “transgenic” organism is an organism having one or more cells that contain an expression vector.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

“Antigen” refers to a molecule capable of being bound by an antibody. An antigen is additionally capable of being recognized by the immune system and/or being capable of inducing a humoral immune response and/or cellular immune response leading to the activation of B- and/or T-lymphocytes. An antigen can have one or more epitopes (B- and/or T-cell epitopes). Antigens as used herein may also be mixtures of several individual antigens. “Antigenic determinant” refers to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

As used herein, the term “antibody” refers to molecules capable of binding an epitope or antigenic determinant. This term includes whole antibodies and antigen-binding fragments thereof, including single-chain antibodies. In certain embodiments, the antibodies are human antigen binding antibody fragments and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. The antibodies can be from any animal origin including birds (e.g. chicken) and mammals (e.g., human, murine, rabbit, goat, guinea pig, camel, horse and the like). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods (Kohler and Milstein, Nature 256:495, 1975) or recombination methods (U.S. Pat. No. 4,816,567). The monoclonal antibodies used in the present invention can be also isolated from a phage antibody library (Clackson et al., Nature 352:624-628, 1991; Marks et al., J Mol. Biol. 222:581-597, 1991). The monoclonal antibodies of the present invention particularly comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention (U.S. Pat. No. 4,816,567; Morrison et al., Proc. Natl. Acad. Sci. USA 81:6851-6855, 1984).

As used herein, the term “mutant antibody” refers to an antibody comprising a variant amino acid sequence in which one or more amino acid residues have been altered. For example, the variable region of an antibody can be modified to improve its biological properties, such as antigen binding. Such modifications can be achieved by site-directed mutagenesis (see Kunkel, Proc. Natl. Acad. Sci. USA 82: 488 (1985)), PCR-based mutagenesis, cassette mutagenesis, and the like. Such mutants comprise an amino acid sequence which is at least 70% identical to the amino acid sequence of a heavy or light chain variable region of the antibody, more preferably at least 75%, even more preferably at least 80%, still more preferably at least 85%, yet more preferably at least 90%, and most preferably at least 95% identical. As used herein, the term “sequence identity” is defined as the percentage of residues identical to those in the antibody's original amino acid sequence, determined after the sequences are aligned and gaps are appropriately introduced to maximize the sequence identity as necessary.

Vaccines of the Invention

The present invention provides a vaccine for use to protect mammals against the colonization and/or infection of certain viruses (e.g., HCV, Ebola or influenza). In one embodiment of this invention, a target protein of the invention can be delivered to a mammal in a pharmacologically acceptable vehicle. As one skilled in the art will appreciate, it is not necessary to use the entire target protein. A selected portion of the target protein can be used, for example the epitope of E2 that specifically binds to CD81.

As one skilled in the art will also appreciate, it is not necessary to use a target protein that is identical to the native target protein. The modified target protein can correspond essentially to the corresponding native protein. As used herein “correspond essentially to” refers to a target protein epitope that will elicit a immunological response at least substantially equivalent to the response generated by a native target protein. An immunological response to a composition or vaccine is the development in the host of a cellular and/or antibody-mediated immune response to the polypeptide or vaccine of interest. Usually, such a response consists of the subject producing antibodies, B cell, helper T cells, suppressor T cells, and/or cytotoxic T cells directed specifically to an antigen or antigens included in the composition or vaccine of interest. Vaccines of the present invention can also include effective amounts of immunological adjuvants, known to enhance an immune response.

To immunize a subject, the target protein, or an immunologically active fragment, variant or mutant thereof, is administered parenterally, usually by intramuscular or subcutaneous injection in an appropriate vehicle. Other modes of administration, however, such as oral, intranasal or intradermal delivery, are also acceptable.

Vaccine formulations will contain an effective amount of the active ingredient in a vehicle, the effective amount being readily determined by one skilled in the art. The active ingredient may typically range from about 1% to about 95% (w/w) of the composition, or even higher or lower if appropriate. The quantity to be administered depends upon factors such as the age, weight and physical condition of the animal or the human subject considered for vaccination. The quantity also depends upon the capacity of the animal's immune system to synthesize antibodies, and the degree of protection desired. Effective dosages can be readily established by one of ordinary skill in the art through routine trials establishing dose response curves. The subject is immunized by administration of the biofilm peptide or fragment thereof in one or more doses. Multiple doses may be administered as is required to maintain a state of immunity to the virus of interest, e.g., HCV, Ebola or influenza.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

To prepare a vaccine, the purified target protein, fragment, or variant thereof, can be isolated, lyophilized and stabilized, as described above. The target protein may then be adjusted to an appropriate concentration, optionally combined with a suitable vaccine adjuvant, and packaged for use. Suitable adjuvants include but are not limited to surfactants, e.g., hexadecylamine, octadecylamine, lysolecithin, dimethyldioctadecylammonium bromide, N,N-dioctadecyl-N′—N-bis(2-hydroxyethyl-propane di-amine), methoxyhexadecyl-glycerol, and pluronic polyols; polanions, e.g., pyran, dextran sulfate, poly IC, polyacrylic acid, carbopol; peptides, e.g., muramyl dipeptide, aimethylglycine, tuftsin, oil emulsions, alum, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993). Finally, the immunogenic product may be incorporated into liposomes for use in a vaccine formulation, or may be conjugated to proteins such as keyhole limpet hemocyanin (KLH) or human serum albumin (HSA) or other polymers.

The application of a target protein described herein or variant thereof, for vaccination of a mammal against certain viruses (e.g., HCV, Ebola or influenza) offers advantages over other vaccine candidates.

Formulations and Methods of Administration

The compositions of the invention may be formulated as pharmaceutical compositions and administered to a mammalian host, such as a human patient, in a variety of forms adapted to the chosen route of administration, i.e., orally, intranasally, intradermally or parenterally, by intravenous, intramuscular, or subcutaneous routes.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound (i.e., target proteins of the present invention) may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use.

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compositions of the present invention in a liquid composition will be from about 0.1-25 wt-%, preferably from about 0.5-10 wt-%.

The amount of the active compound required for use in treatment will vary with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The active compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day.

The invention will now be illustrated by the following non-limiting Examples.

### Example 1

### Molecular Chaperones of the Endoplasmic Reticulum to Promote Recombinant Protein Production in Plants

Infections caused by the Hepatitis C Virus (HCV) are very common worldwide, affecting up to 3% of the population. Chronic infection of HCV may develop into liver cirrhosis and liver cancer, which is among the top five of the most common cancers. Therefore, vaccines against HCV are under intense study in order to prevent HCV from harming people's health. The envelope protein 2 (E2) of HCV is thought to be a promising vaccine candidate because it can directly bind to a human cell receptor and plays a role in viral entry. However, the E2 protein production in cells is inefficient due to its complicated matured structure. Folding of E2 in the endoplasmic reticulum (ER) is often error-prone, resulting in production of aggregates and misfolded proteins. These incorrect forms of E2 are not functional because they are not able to bind to human cells and stimulate antibody response to inhibit this binding.

Described herein are studies aimed at overcoming the difficulties of HCV E2 production in a plant system. Protein folding in the ER requires great assistance from molecular chaperones. Thus, in this study, two molecular chaperones in the ER, calreticulin and calnexin, were transiently overexpressed in plant leaves in order to facilitate E2 folding and production. Both of them showed benefits in increasing the yield of E2 and improving the quality of E2. In addition, poorly folded E2 accumulated in the ER may cause stress in the ER and trigger transcriptional activation of ER molecular chaperones. Therefore, a transcription factor involved in this pathway, named bZIP60, was also overexpressed in plant leaves, aiming at up-regulating a major family of molecular chaperones called BiP to assist protein folding. However, the results described herein showed that BiP mRNA levels were not up-regulated by bZIP60, but they increased in response to E2 expression. The Western blot analysis also showed that overexpression of bZIP60 had a small effect on promoting E2 folding. Overall, this study suggested that increasing the level of specific ER molecular chaperones was an effective way to promote HCV E2 protein production and maturation.

The following abbreviations are used herein: activating transcription factor 6 (ATF6); binding immunoglobulin protein (BiP); luminal binding protein (Blp); basic Leucine Zipper protein (bZIP); C terminal truncated basic Leucine Zipper protein 60 (bZIP60ΔC); Cluster of Differentiation 81 (CD81); cauliflower mosaic virus (CaMV); calnexin (CNX); calreticulin (CRT); days post-infiltration (dpi); Dithiothreitol (DTT); HCV envelope protein 1 (E1); HCV envelope protein 2 (E2); Ethylenediaminetetraacetic acid (EDTA); elongation factor 1α (EF1α); eukaryotic translation initiation factor 2 (eIF2); endoplasmic reticulum (ER); endoplasmic reticulum stress response element (ERSE); glycoprotein (GP); Hepatitis C virus (HCV); horseradish peroxidase (HRP); immunoglobulin G (IgG); inositol-requiring enzyme 1 (IRE1); kilo Dalton (kDa); left border (LB); long intergenic region (LIR); membrane bound envelope protein 2 (mE2); messenger ribonucleic acid (mRNA); nopaline synthase (NOS); open reading frame (ORF); phosphate-buffered saline containing 0.05% Tween 20 (PBST); kinase R-like ER kinase (PERK); kinase R (PKR); plant—unfolded protein response element (p-UPRE); right border (RB); replication initiator protein (Rep); sodium dodecyl sulfate polyacrylamide gel electrophoresis (SDS-PAGE); soluble form of envelope protein 2 (sE2); short intergenic region (SIR); transferred deoxyribonucleic acid (T-DNA); tobacco etch virus (TEV); tobacco mosaic virus (TMV); unfolded protein response (UPR).

Vaccination is currently regarded as the most effective way of preventing infectious diseases by the public. Indeed, many good vaccines such as influenza vaccines and the Hepatitis B virus vaccine work very well in preventing their targeted viral infections, saving thousands of peoples' lives. Therefore, in order to better protect the public from infectious diseases, more efforts are being made to develop new effective and safe vaccines against more infectious diseases, especially those that are lethal but lack prevention methods. A recombinant viral protein vaccine is one type of vaccine that uses protein components of a virus which are immunogenic but not infectious to induce immune responses in the host. They are considered safer than live or killed virus vaccines because they lack the viral nucleic acid which is responsible for viral replication. Recombinant viral proteins are mostly produced in bacteria, yeast or mammalian cells. However, sometimes these systems have their own shortages. For example, bacteria cannot produce glycosylated proteins because they lack this post-translational modification process. But a large portion of viral proteins used as vaccines are glycosylated, such as viral envelope proteins found on the outmost surface of viruses. Plants are a relatively new system used for recombinant protein vaccine production. The advantages of using a plant expression system include rapid protein expression, easy and safe manipulation, and low cost of vaccine production and manufacturing.

The goal of the studies described herein is to efficiently produce the functional envelope protein E2 of HCV using a plant expression system, in order to help the development of a recombinant protein vaccine against HCV. Previous studies have showed that E2 protein is often folded poorly in the Endoplasmic Reticulum (ER), reducing the yield of native form of E2. The strategy used in this study is to increase the levels of several molecular chaperones in the ER which are responsible for helping glycoprotein to fold, thereby enhancing the ER's ability to fold newly synthesized or misfolded E2 polypeptides into native proteins. The ER molecular chaperones are thought to have functions in preventing intramolecular or intermolecular aggregation, suppressing pre-matured protein degradation, and facilitating ER folding factors to catalyze protein folding. As described herein, increasing ER molecular chaperone levels can help to improve the quality and the quantity of HCV E2 produced in this plant system. The experiments discussed below involve either overexpressing specific ER molecular chaperones involved in glycoprotein folding, or overexpressing a transcription factor that is thought to activate several genes encoding ER chaperones and ER folding factors. The resulting effects on HCV E2 folding and production were tested, and the results indicated that they did promote HCV E2 production in the ER.

Though HCV E2 is likely to be a potent HCV vaccine candidate, inefficient folding of E2 in the ER has become a big problem in recombinant E2 vaccine development. Experiments on recombinant E2 expression have shown that E2 expression often results in significant intermolecular aggregation which is stabilized by intermolecular disulfide bond (1). Those high-molecular-weight E2 aggregates are not the active form of E2, but they occupy a large portion of the products. Studies on their structure and functions are very limited, and whether or not they can induce an antibody response in the host is unknown. But, at least it is known that E2 aggregates bind poorly to CD81, the putative receptor of E2 broadly expressed on human cells (2). This means that those aggregates do not have the binding site to CD81 on the cell surface. Therefore, even if there are antibodies against E2 aggregates, they cannot effectively block the entry of HCV into human cells. In a word, aggregation is not desired in recombinant E2 protein production; new methods are needed to improve the E2 folding pathway in cells, so that a more correct form of E2 to make vaccines can be acquired.

An aim of this work is to increase the production of properly folded HCV E2 proteins in a plant system. Accordingly, several ER molecular chaperones were overexpressed in plants to promote HCV E2 folding. These experiments identified ER molecular chaperones that are important for facilitating HCV E2 folding, and enabled a better understanding of their roles in E2 processing. Improved folding of the E2 protein could greatly benefit HCV E2 vaccine development because it would save time and labor, and therefore, would lower the total cost for HCV E2 vaccine production. This method of improving protein folding by increasing ER chaperone levels may also be applied to other viral glycoprotein productions in plants as well. Since many viral envelope proteins are glycoproteins and also major antigens to the host during infection, this strategy may benefit vaccine development against other viruses as well.

### Hepatitis C Virus and its Envelope Protein Vaccine Development

Hepatitis C Virus (HCV) is a single-stranded, positive-sense RNA virus that causes infection in the liver, leading to strong inflammation. Chronic HCV infection may develop into liver cirrhosis and hepatocellular carcinoma (liver cancer) (3, 4). It affects about 200 million people worldwide with 3 to 4 million new cases per year, as reported by World Health Organization. HCV is mainly transmitted by exposure to contaminated blood, and more than 60% of the infected people are not able to fully recover from infection and become chronic carriers. Unfortunately, no vaccines against HCV are available for prevention and treatment so far. Therefore, HCV vaccines are currently in urgent need, and different strategies are being tried for HCV vaccine development.

To date, primary results from animal studies show that recombinant HCV envelope proteins are promising HCV vaccine candidates because some of them can induce relatively strong antibody responses which are able to protect the host from subsequent challenge with the homologous virus (4, 5). HCV genome encodes 2 envelope proteins named E1 (gp31) and E2 (gp70). E1 and E2 are both Asparagine-linked glycoproteins (N-linked glycoproteins) and they form heterodimers on the surface of HCV, serving as major antigens which can be recognized by the immune system of the host. E1 or E2 protein alone is also antigenic. Studies have shown that the E2 protein can bind to a cell receptor called CD81 on human cells, and this interaction can be blocked by anti-E2 antibodies produced from sera of animal model in vitro (6). This suggested that binding of E2 to CD81 may be relevant to HCV infection. Hence, E2 becomes a promising vaccine candidate to prevent HCV infection since it is likely to induce generation of naturalizing antibodies that can protect the host by inhibiting HCV's entry to host cells. However, a big challenge to develop HCV E2 vaccine is that it is difficult to produce sufficient amount of properly folded E2 proteins, mainly because of their complicated mature structure and heavy glycosylation modifications. It is known that an antigen with incorrect structure may lose its ability to stimulate host immune cells to produce antibodies against it. Thereby, researchers are making effort to create several truncated forms of E2 in order to simplify the structure of E2 while maintaining its immunogenicity (2). Optimization of the expression systems that express E2 is another strategy to increase the yield of correctly folded E2.

### Roles of ER Chaperons in Glycoprotein Folding

In order to become a functionally active protein, newly synthesized polypeptides must undergo folding and assembly in the endoplasmic reticulum (ER) to obtain a unique native structure. This process is usually coordinated with post-translational modifications such as N-linked glycosylation and disulfide bond formation. An incorrect structure of a protein may disable the protein to interact with other molecules and play its function. Therefore, efficient protein folding is significantly essential to produce a functional protein. However, in the ER, nascent polypeptide chains are very likely to misfold and aggregate themselves, especially for those proteins whose mature structures are very complex. A big reason for that is protein folding is coupled with protein synthesis. Since synthesis of protein is a sequential process (from N-terminus to C-terminus), it is possible that polypeptides near the N-terminus already folded into an incorrect structure before the complete folding information encoded in a polypeptide chain is available. Besides the inherent complicated structure of protein, the efficiency of protein folding can also be strongly reduced by high concentration of macromolecules in the ER after protein translation, leading to a crowded environment which favors intermolecular associations among polypeptide chains. This hypothesis has already been confirmed experimentally; the result indicated that the folding rate of protein decreased and the danger of aggregation increased (7). Fortunately, the lumen of the ER contains many molecular chaperones which are designed to facilitate protein folding by increasing the efficiency of the folding process. They transiently bind to nascent and incompletely folded polypeptide chains, and release them in a regulated manner, preventing them from incorrect interactions. Therefore, the role of ER chaperones is thought to be preventing the tendency of aggregation between non-native polypeptide chains, thereby ensuring efficient protein folding (8).

In the ER, there are different types of molecular chaperones that help protein folding, including general chaperones, lectin chaperones and non-classical chaperones. Among them, two lectin chaperons, calnexin and calreticulin, are major chaperones specifically facilitating glycoprotein folding. Calnexin is an ER resident membrane protein, and calreticulin is its soluble homolog in the ER lumen (9, 10). They preferentially and transiently associate with newly synthesized N-linked glycoproteins in a regulated manner, mainly due to their lectin-like affinity for monoglucosylated oligosaccharides (Glc1Man9GlcNAc2) found on pre-mature N-linked glycoproteins (11). Calnexin and calreticulin do not have a binding site on the correctly folded matured glycoprotein. After proteins are synthesized in the ER, glycans with three external glucose residues are linked to the asparagine residues of nascent proteins. The three glucose molecules are then trimmed by ER located enzyme glucosidase I and glucosidase II sequentially to make a mature glycoprotein that will be exported from the ER. Therefore, only the processing intermediate containing one glucose molecule can be recognized by calnexin and calreticulin. If a glycoprotein is misfolded, an ER-resident enzyme called UDP-glucose:glycoprotein glucosyltransferase (UGGT) can reglucosylate the N-linked glycan so that the glycoprotein can be re-associated with calnexin and calreticulin. How this binding cycle promotes glycoprotein folding is yet to be studied. Some studies on folding of influenza hemagglutinin (HA), which is also an N-linked glycoprotein, demonstrated that calnexin and calreticulin bound to different but overlapping folding intermediates of influenza HA, slowing down the protein folding and assembly process, but increased the overall efficiency of HA maturation because of less aggregation and degradation of HA. They suggested that calnexin and calreticulin promoted protein folding by facilitating retention of misfolded proteins in the ER, and by preventing aggregation and degradation of incompletely folded proteins (12, 13).

Another important ER chaperon that helps glycoprotein folding is called Binding immunoglobulin protein (BiP), which is also called 78 kDa glucose-regulated protein (GRP-78) or heat shock 70 kDa protein 5. It is a general ER chaperone, so it does not specifically modulate glycoprotein folding. However, BiP is a central stress regulator of the ER. The expression of BiP protein can be remarkably induced by accumulation of unfolded or misfolded proteins in the ER, in order to promote protein folding and oligomerization. As other heat shock 70 kDa proteins, BiP is an ATPase which couples ATP hydrolysis to the binding and release of proteins. BiP has a peptide binding domain at C-terminus and an ATPase domain at N-terminus. When the ATPase domain of BiP interacts with ATP and triggers hydrolysis, a conformational change occurs at its C-terminal peptide binding domain and allows it to bind to unfolded or misfolded proteins. BiPs are thought to have high affinity binding to hydrophobic regions that are exposed by non-native proteins. Under this condition, protein disulfide isomerase in the ER can come to catalyze incorrect disulfide bond reduction and correct disulfide bond formation of the trapped protein. After that, exchange from ADP to ATP at the N-terminus of BiP results in releasing the refolded protein to the ER environment (14). A corrected folded protein will no longer be targeted by BiP proteins. Hence, in regards to helping protein folding, the function of BiP is to stabilize the non-native structures of proteins until they can undergo subsequent folding, and to minimize incorrect interaction between molecules by shielding exposed hydrophobic regions of polypeptides.

Overall, calnexin, calreticulin and BiP have their own roles to promote glycoprotein folding in the ER. Particularly, the lectin binding site of calnexin was shown to have a significant advantage over BiP in suppressing aggregation of glycoproteins, indicating the importance of lectin-glycan binding in facilitating glycoprotein folding (15).

### ER Stress Response in Plants

ER stress response, also known as unfolded protein response (UPR), is a conserved mechanism used by all eukaryotic cells to relieve the “ER stress” caused by accumulation of unfolded or misfolded proteins in the ER lumen (16). It triggers the protein quality control system to attenuate global protein translation and degrade proteins in the ER. It also induces a signaling pathway that result in up-regulation of ER molecular chaperones to promote protein refolding. If the ER stress cannot be relieved by these actions, the programmed cell death will be activated. In mammalian cells, ER stress is generally sensed by three transmembrane proteins located in the ER: protein kinase R (PKR)-like ER kinase (PERK), activating transcription factor 6 (ATF6), and inositol-requiring enzyme 1 (IRE1) (17, 18). Under stress condition, the three sensors are activated to perform their functions in ER stress response. PERK acts on the eukaryotic translation initiation factor 2 (eIF2), leading to attenuation of protein translation (19). ATF6 moves to Golgi bodies and is cleaved by S₁P and S₂P proteases there, releasing its N-terminal domain to the nucleus to activate UPR genes such as genes encoding ER chaperone (20). IRE1 not only directs UPR genes activation but also specifically induces protein degradation (21).

In plants, although little information about ER stress response is available, two IRE1-like proteins and two ATF6-like stress transducers bZIP60 and bZIP28 have been identified (22). The function of plant IRE 1 proteins as transcription inducer is yet to be determined, but the functions of basic leucine zipper (bZIP) transcription factor 60 and 28 have recently been characterized in Arabidopsis (23, 24). Both Arabidopsis bZIP60 (AtbZIP60) and bZIP28 (AtbZIP28) are type II transmembrane proteins localized in the ER membrane when they are inactive. When cells are stressed, they are activated by proteolytic cleavage of N-terminal domain at cytoplasmic side, and the free active forms of AtbZIP60 and AtbZIP28 are then translocated to the nucleus, at where they function as transcription factors to induce expression of multiple UPR genes, including BiP, by binding to the ER stress response element (ERSE) or plant UPR element (p-UPRE) in their promoters (23, 24). Although both AtbZIP60 and AtbZIP28 are activated in response to ER stress, the activation of AtbZIP60 is much stronger than AtbZIP28. In addition, AtbZIP60 and AtbZIP28 proteins show little homology. Furthermore, AtbZIP28 contains S₁P and S₂P protease sites in its protein sequence, which suggests that it is cleaved by S₁P/S₂P system in the Golgi apparatus. Nevertheless, bZIP60 does not contain S₁P and S₂P sites, and its cleavage is not affected by mutations in the genes encoding Arabidopsis S₁P and S₂P proteases, indicating a different cleavage mechanism (25). Recently, Chika Tateda (2008) and his group reported a homolog of AtbZIP60 found in Nicotiana tabacum, named NtbZIP60 (26). The study showed that it had similar functions to AtbZIP60. Activated by ER stress, its N-terminal domain was cleaved and released from ER membrane, targeting to the nucleus. It could also transactivate the reporter gene containing p-UPRE cis-elements.

### HCV Envelope Protein E2 Production in the ER

HCV envelope protein E2 is an N-linked glycoprotein with 11 glycosylation sites. It interacts non-covalently with the other HCV envelope protein E1 to form a heterodimer on the surface of HCV (27). Due to the complicated 3D structures of E2, its folding process and the subsequent E1-E2 complex assembly process in the ER is rather slow and error-prone. Generally, significant aggregation and consequent degradation of unfolded and misfolded E2 proteins are shown during E2 maturation, which reduces the folding efficiency and increases the ER stress. Some studies suggested that this tendency of aggregation was intrinsic, not because of over-production of E2 proteins in the ER (28). Interaction of E2 with calnexin, calreticulin and BiP has already been reported in mammalian cells, suggesting important roles of the three ER chaperones in helping E2 folding (29). However, over-expression of each chaperone did not increase the level of native E1-E2 complexes in that study. Another research showed that high level of E2 could modulate the ER stress response by inhibiting the PERK pathway induced protein translational attenuation, so that it could promote its own synthesis (19). As a result, large amounts of non-native proteins together with inefficient protein folding make HCV envelope protein E2 very toxic to host cells.

HCV E2 protein is a slow-folded glycoprotein and seems to have an intrinsic tendency of aggregation. Hence, finding ways to increase its folding efficiency so that more non-aggregated and functional E2 proteins can be acquired when it is expressed in plants is desired. Since folding of proteins requires important assistance from the ER molecular chaperones, it is hypothesized that overexpression of the ER molecular chaperones that are particularly essential to help glycoprotein folding will help to express more functional HCV E2 by increasing the efficiency of protein folding. Such molecular chaperones in the ER include calnexin and calreticulin. These chaperones are advantageous over other ER molecular chaperones for the facilitation of glycoprotein folding mainly due to their lectin sites with a high affinity for the N-linked glycan side chains of glycoproteins. This interaction allows calnexin and calreticulin to suppress aggregation of some glycoproteins more effectively than other general molecular chaperones (15). Therefore, according to this hypothesis, it is predicted that overexpression of calnexin, calreticulin or both of them in the plant expression system will increase the yield of E2 and improve the quality of E2.

In addition to the overexpression of those two particular molecular chaperones, induction of several chaperons expressions involved in UPR by bZIP60 may also have a significant effect on folding nascent polypeptide chains and refolding the misfolded proteins. This is because bZIP60 can activate many ER molecules responsible for protein folding at the same time, including BiP and calnexin, according to the studies done in Arabidopsis (30). These molecules can work together to promote protein folding to reduce the stress in cells. Hence, a second hypothesis is that overexpression of bZIP60 in plant cells will make those cells more sensitive to the ER stress generated by E2 expression, and immediately induce more UPR genes expression such as BiP to participate the folding of newly synthesized and misfolded E2, therefore increasing the amount of properly folded E2 proteins.

### Materials and Methodology

### Research Design

Although calnexin and calreticulin share functions in glycoprotein folding, a major distinction between them is that calnexin is a membrane protein but calreticulin is soluble in the ER lumen. This may have an effect on the type of protein they interact with, because some studies indicated that calnexin preferentially associated with membrane bound protein-folding intermediates rather than their truncated soluble forms (31). Therefore, it was decided to test the first hypothesis on two versions of recombinant E2 proteins, one is a truncated soluble version lacking the transmembrane domain (sE2), and the other is a full-length insoluble version containing the membrane anchor (mE2). It was planned to co-express Arabidopsis calreticulin and sE2 in leaves of Nicotiana benthamiana, which is the plant model that was used to express HCV E2 by transient transformation, and compare the resulting amount of total sE2 and properly folded sE2 to that from plant leaves expressing sE2 alone by Western blot. The same strategy also applied to co-expression of Arabidopsis calnexin and mE2, and the level of total and non-aggregated mE2 were measured and compared to that from leaves that only express mE2.

To test the second hypothesis, bZIP60 cDNA was cloned from wild type N. benthamiana leaves to make the DNA construct overexpressing N. benthamiana bZIP60 (NbbZIP60). The same method was also used to generate a DNA construct overexpressing the putative active form of NbbZIP60 which did not have the transmembrane domain and the C-terminal domain. The rationale is that the truncated NbbZIP60 (NbbZIP60ΔC) may activate the targeted UPR genes more efficiently because they are free to enter the nucleus, independent of ER stress. The NbbZIP60 or NbbZIP60ΔC construct was then transformed into plant leaves together with the construct expressing soluble form of E2. The leaves expressing these transgenes would transiently have an increased level of NbbZIP60 or NbbZIP60ΔC in the ER and also high level of the soluble HCV E2 proteins. Then it could be determine the effect of overexpression of NbbZIP60 or NbbZIP60ΔC on E2 folding and production by comparing E2 protein level in NbbZIP60 overexpressed and normally expressed leaves. The quantity and quality of E2 produced in NbbZIP60 overexpressed leaves could also be compared to that produced in NbbZIP60ΔC overexpressed leaves, so that it could be determined whether their effects on HCV E2 folding and accumulation are different. In addition to the usage of NbbZIP60 and NbbZIP60ΔC, AtbZIP60 and AtbZIP60ΔC were also tested in the same way to examine the effects on HCV E2 folding and production.

Construction of Expression Vectors Used in this Study

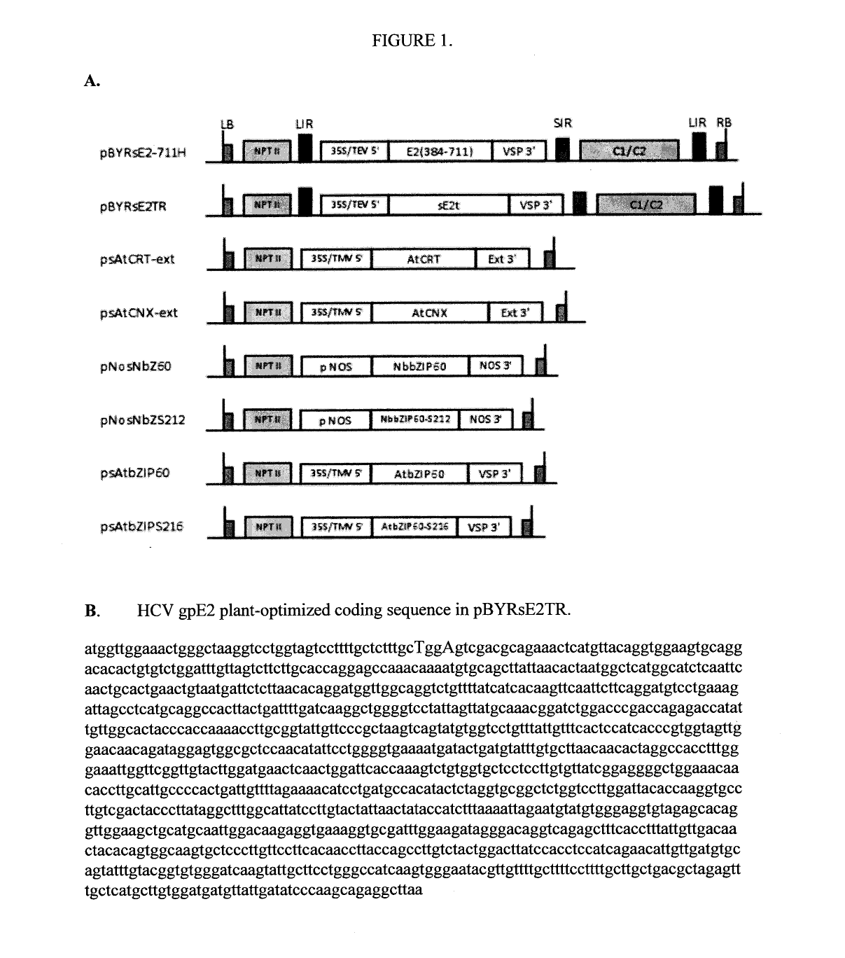

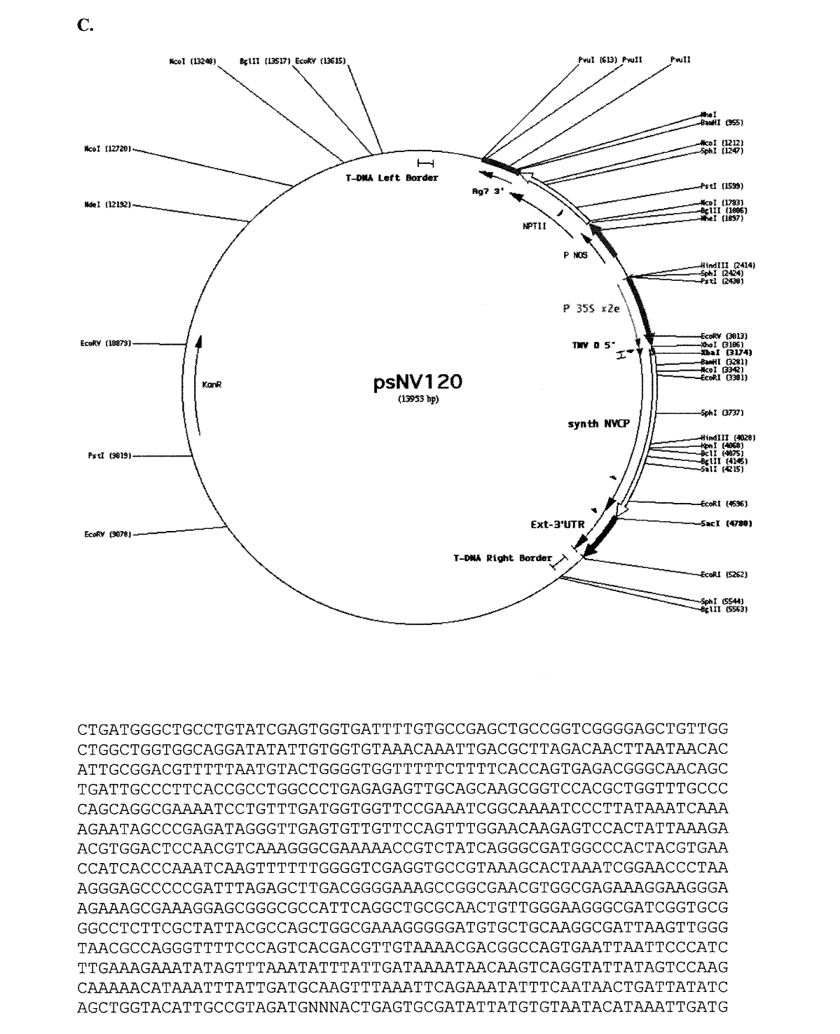

In this study, a soluble form of HCV E2 and a membrane anchored HCV E2 were constructed in germiniviral replicon vectors. Calreticulin, calnexin, bZIP60 and bZIP60ΔC from Arabidopsis, and bZIP60 and bZIP60ΔC from N. benthamiana were constructed in non-viral vectors. A schematic representation of the T-DNA region of the vectors was shown in FIG. 1A.

Construction of Germiniviral vectors.

pBYRsE2-711H contains the HCV E2 coding sequence truncated to use residues 384-711 of the HCV polyprotein, with a sequence encoding the peptide “HHHHHHDEL” added to its C-terminus. It contains the native HCV E2 signal peptide at its N-terminus The plant-optimized coding sequence was based upon the native HCV sequence (Genbank accession M62321) and designed to use codons preferred by N. tabacum and to remove spurious mRNA processing signals. The coding sequence was amplified by high-fidelity PCR using primers sE2-Xba-F (5′-agcttctagaacaatggttggaaactggg) and sE2-711-Nhe-R (5′-cccgctagcaatacttgatcccacac) to create XbaI at 5′ and NheI at 3′, and ligated with annealed oligonucleotides Nhe-6HDEL-Sac-F (5′-ctagccaccatcaccatcaccatgacgagctttaagagct) and Nhe-6HDEL-Sac-R (5′-cttaaagctcgtcatggtgatggtgatggtgg). The resulting coding sequence was inserted into a geminiviral replicon pBYR1 similar to pBYGFP.R (32).

pBYRsE2TR contains the full-length HCV E2 coding sequence for residues 384-746 of the HCV polyprotein, including the C-terminal membrane anchor domain. It contains the native HCV E2 signal peptide at its N-terminus. The plant-optimized coding sequence (FIG. 1B) was inserted into a geminiviral replicon vector pBYR1 to produce pBYRsE2TR.

Construction of ER Charperone Vectors.

The coding sequence of Arabidopsis calreticulin (AtCRT, Genebank accession number NM_{—}104513) was amplified by high-fidelity PCR using primers AtCRT-Xba-F (5′-cctctagaacaatggcgaaactaaaccctaaa) and AtCRT-Kpn-R (5′-ggGGTACCttaaagctegtcatgggcg) on the template pUNI-15759 (obtained from The Arabidopsis Information Resource Center, http://www.arabidopsis.org/index.jsp, stock U15759), digested with XbaI and KpnI and inserted into a geminiviral vector pBYR2, to yield pBYR-AtCRT. The coding sequence was released from pBYR-AtCRT by digestion with XbaI and SacI restriction enzymes, and inserted into the binary vector psNV 120 (FIG. 1C) to yield the vector psAtCRT-ext. The resulting AtCRT expression cassette contained the double enhancer cauliflower mosaic virus (CAMV) 35S promoter (2×35S) with tobacco mosaic virus (TMV) 5′UTR, AtCRT coding sequence and tobacco extensin 3′ UTR.

To obtain Arabidopsis calnexin (AtCNX) expression vector, the cDNA region of AtCNX (Genebank accession number NM_{—}120816) was amplified from pENTR-(TAIR stock U16625, Genbank accession AY059880) with primers AtCNX-Xba-F (5′-cctctagaacaatgagacaacggcaactattttc) and AtCNX-Kpn-R (5′-ggggtaccttgttctaattatcacgtctcg), digested with XbaI and KpnI, and inserted into the geminiviral replicon vector pBYR2, to yield pBYR-AtCNX. The coding seqeunce was released from pBYR-AtCNX by digestion with XbaI and KpnI, and the resulting fragment was inserted into the psAtCRT-ext vector at the corresponding digestion sites, replacing the AtCRT fragment to yield psAtCNX-ext.

For overexpression of NbbZIP60 and NbbZIP60ΔC, cDNA regions encoding full length of NbbZIP60 and truncated NbbZIP60 (amino acid positions 1-212) were amplified by Phusion® high-fidelity DNA polymerase (FINNZYMES) in PCR from total cDNA of wild type N. benthamiana leaves. The primers (see Table 1, primer list) for NbbZIP60 amplification were NbbZIP60-Nco-F which added an NcoI site at the 5′ end, and NbbZIP60-SacI-R which added a SacI site at the 3′ end. The primers for NbbZIP60ΔC amplification were NbbZIP60-Nco-F and NbbZIP60-S212 which added a SacI site at the 3′ end. Since the coding sequence of NbbZIP60 is not deposited in the Genebank, the primers were designed according to the NtbZIP60 cDNA sequence (Genebank accession number AB281271) which was thought to share more than 96% sequence homology with NbbZIP60 (26). The PCR products were digested by NcoI and SacI restriction enzymes and then inserted into pIBT210.3 (33) at NcoI and SacI sites respectively. The resulting constructs were sperately transformed into E. coli DH5α competent cells by electroporation to confirm the insertion and send for sequencing. Sequencing result showed that the NbbZIP60 that was obtained had a 95% homology to the N. tobaccum bZIP60 cDNA sequence. The constructs were then digested with NcoI and SacI restriction enzymes, releasing the NbbZIP60 and NbbZIP60ΔC fragments. The binary vector pGPTV-Kan (34) was digested with BamHI (blunted by filling in with Klenow enzyme) and SacI restriction enzymes, and ligated with the NbbZIP60 or NbbZIP60ΔC NcoI-SacI fragment and the PvuII-NcoI fragment from pGPTV-Kan containing the nopaline synthase (Nos) promoter, thus yielding plasmids with the coding sequences between Nos promoter and Nos 3′ UTR, and they were called pNosNbZ60 and pNosNbZS212.

The coding sequences of AtbZIP60 and AtbZIP60ΔC (amino acid 1-216) are shown in FIGS. 1D and 1E, respectively. For AtbZIP60 and AtbZIP60ΔC expression vectors, cDNA regions encoding AtbZIP60 (full length) and AtbZIP60ΔC (truncated, 1-216) were amplified from the vector pUni51-AtbZIP60 (TAIR stock number 4775801) by Phusion® high-fidelity DNA polymerase in PCR, using the primer pUni51-F and AtbZIP60-Kpn-R which added a KpnI site at the 3′ end for AtbZIP60, and the primer pUni51-F and AtbZIP60-S216-K which added a KpnI site at the 3′ end for AtbZIP60ΔC. The vector pIBT210.3 and the PCR products were respectively digested by NcoI and KpnI restriction enzymes, and the resulting AtbZIP60 and AtbZIP60ΔC fragments were separately ligated to pIBT210.3. The resulting constructs were transformed to E. coli DH5α competent cells and verified by PCR and digestion by NcoI and KpnI enzymes. After that, the AtbZIP60 and AtbZIP60ΔC fragments were released from the constructs by NcoI and KpnI restriction digestion and inserted into a binary vector pPS1 respectively at the corresponding restriction sites, yielding psAtbZIP60 and psAtbZIPS216. The expression cassette contained the double enhancer cauliflower mosaic virus (CAMV) 35S promoter (2×35S), tobacco mosaic virus (TMV) 5′UTR, AtbZIP60 or AtbZIP60ΔC cDNA sequence, and soybean vspB gene 3′ element.

### Plant Materials and Agroinfiltration of Expression Vectors

Germiniviral vectors and non-replicating binary vectors were separately introduced into Agrobacterium tumefaciens strain GV3101 by electroporation. The resulting strains were verified by colony screening using PCR and restriction digestion of plasmids. Then they were grown in liquid culture medium for 1 to 2 days to be ready for agroinfiltration. 6 to 7 weeks old greenhouse-grown N. benthamiana plants were used as the expression host. For infiltration, the Agrobacteria were spun down by centrifugation at 5000 rpm for 6 min and resuspended in infiltration buffer (10 mM 2-(N-morpholino)ethanesulfonic acid (MES), pH 5.5 and 10 mM MgSO₄) to OD₆₀₀=0.2. The plant leaves were then inoculated with one or mixed Agrobacterium strains by needle infiltration. The agroinfiltration procedure was performed as previously described (32). Infiltrated plants were maintained in a growth chamber for several days to allow transgene expression.

### RNA Extraction and Reverse Transcription Polymerase Chain Reaction (RT-PCR)

Total RNA were extracted from infiltrated plant leaves 48 h after infiltration, using a plant RNA purification reagent (invitrogen) and chloroform:isoamyl-alcohol (24:1). Then the RNA was precipitated in isopropyl alcohol at room temperature for 10 min. The RNA pellet was washed with 75% ethanol and resuspended in 50 μl DEPC-treated water. The residual DNA in the RNA sample could be removed by DNase included in the TURBO DNA-free™ system (Ambion) according to the manufacturer's instruction.

To perform RT-PCR, first-strand cDNA were synthesized from 1 μg purified total RNA using Oligo(dT)₂₀ primer included in the SuperScript™ III First-Strand Synthesis System for RT-PCR (invitrogen), according to the manufacturer's instruction. 2 μl of cDNA sample were directly used as templates in the PCR to amplify desired transcripts using gene-specific primer sets. RNA without reverse transcriptase was also amplified by PCR to confirm no genomic DNA contamination in samples.

### Protein Extraction and Western Blot

Soluble proteins were extracted by grinding 100 to 200 mg of leaf sample in 0.5 ml extraction buffer (20 mM Tris(pH8.0), 20 mM KCl, 1 mM EDTA, 0.1% Triton X-100, 50 mM Sodium Ascorbate, 10 μg/ml Leupeptin) using the bullet Blender® (Next Advance). The resulting leaf crude extracts were held on ice for 1 h to allow full extraction. Then they were centrifuged at 12,000 rpm and 4° C. for 15 min and the supernatants were transferred to new tubes for subsequent analysis by Western blot. To extract proteins in the pellet of leaf crude extracts, the same amount of the extraction buffer was added to the pellet to resuspend it. Total protein amount in a sample was determined by the Bradford assay (BIO-RAD). Usually, 15 μg of proteins per sample were added in the SDS-PAGE sample buffer either with 150 mM DTT reducing reagent or without it, and then loaded onto 4-15% gradient polyacrylamide gels for separation. Equivalent loading of total proteins in each sample to the gel was determined by Coomassie blue staining of the gel, and proteins separated on the gel could also be transferred to a polyvinlidene difluoride (PVDF) membrane (Amersham, N.J.) for Western blot analysis. To detect denatured E2 proteins, the membrane was incubated with mouse monoclonal anti-E2 antibody against a linear epitope (Chiron/Novartis) diluted at 1:10000 in 1% skim milk in PBST at 37° C. for 1 h, after washing the membrane with PBST for 4 times, the membrane was then incubated with goat anti-mouse IgG-horseradish peroxidase (HRP) conjugate (Sigma) diluted at 1:5000 in 1% skim milk in PBST at 37° C. for another hour. To detect conformational E2 proteins, the membrane was probed with mouse monoclonal anti-E2 antibody against a conformational epitope (Chiron/Novartis) diluted at 1:5000 in 1% skim milk in PBST at 37° C. for 1 h, and then they were washed with PBST for 4 times and detected with goat anti-mouse IgG-HRP conjugate diluted at 1:5000 in 1% skim milk in PBST at 37° C. for another hour. Finally, the membranes were washed again with PBST for 4 times and developed by chemiluminescence using ECL plus detection reagent (Amersham, N.J.).

HCV E2 Transient Expression in Nicotiana benthamiana Leaves

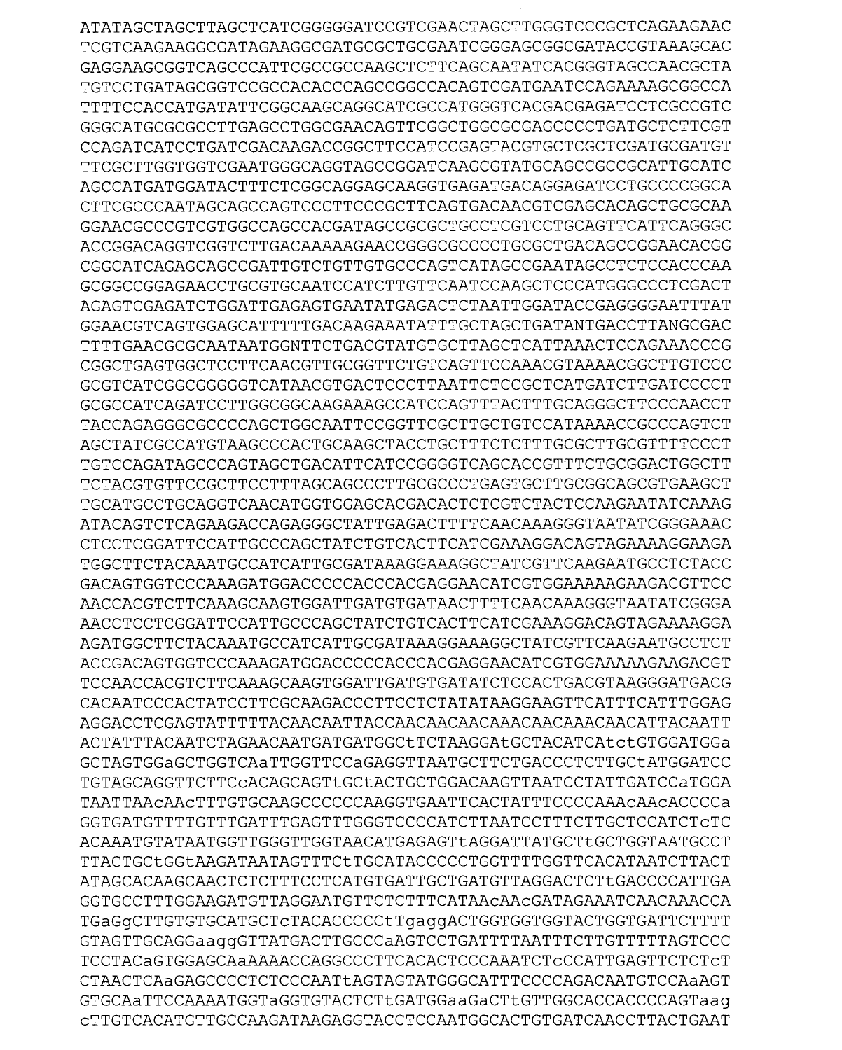

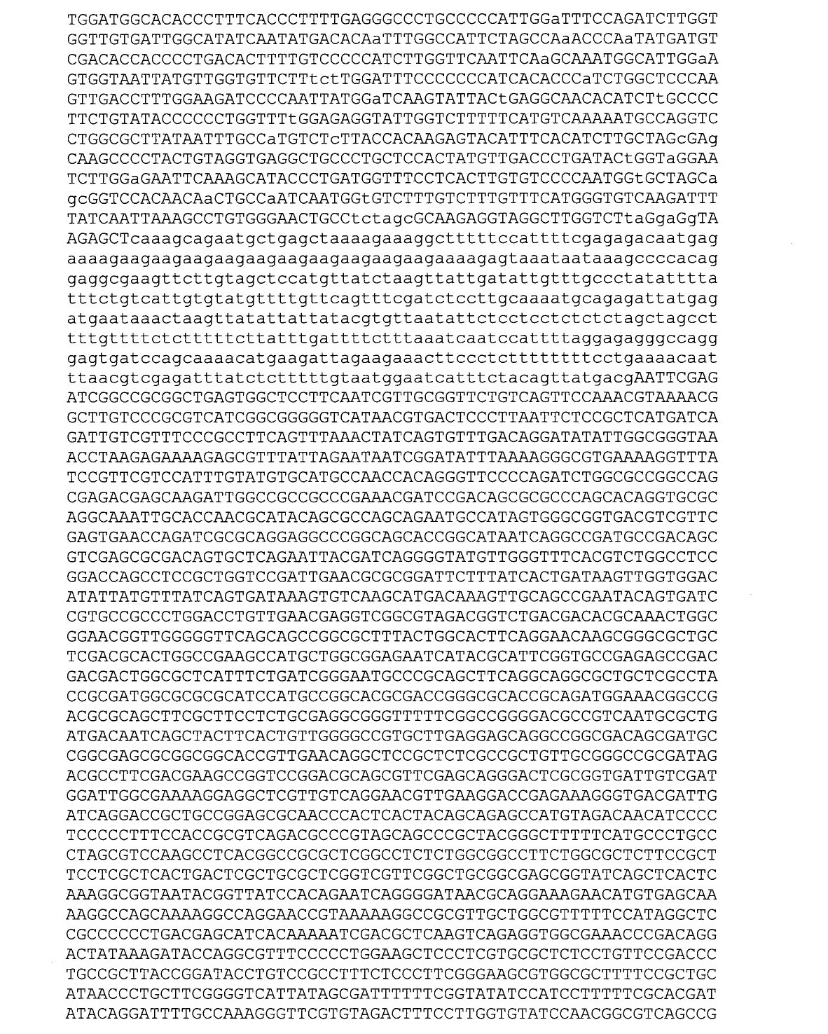

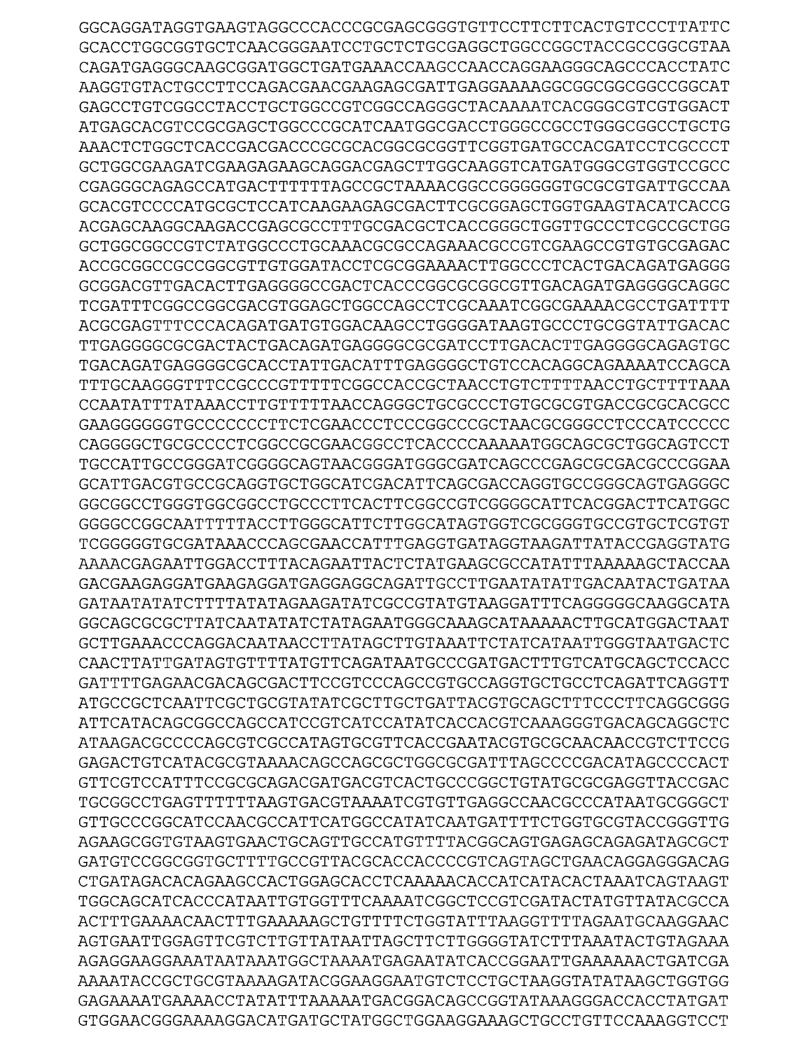

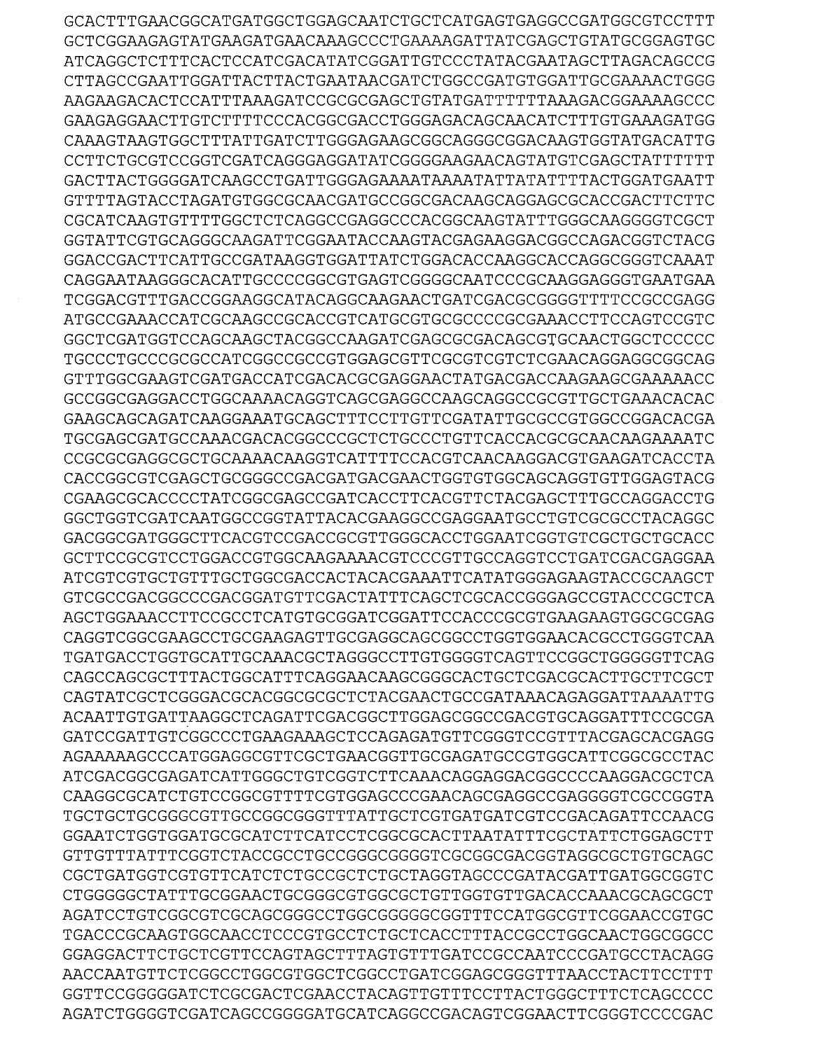

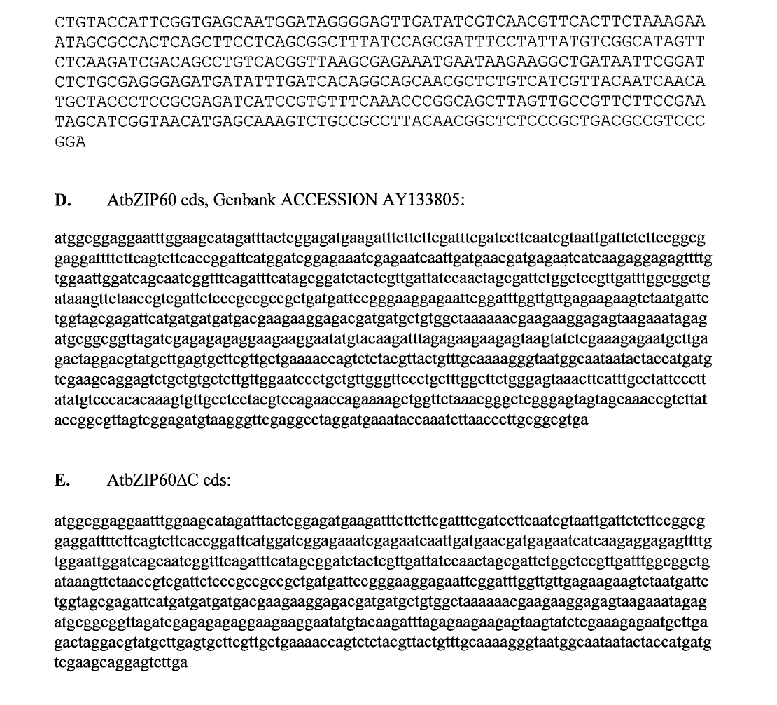

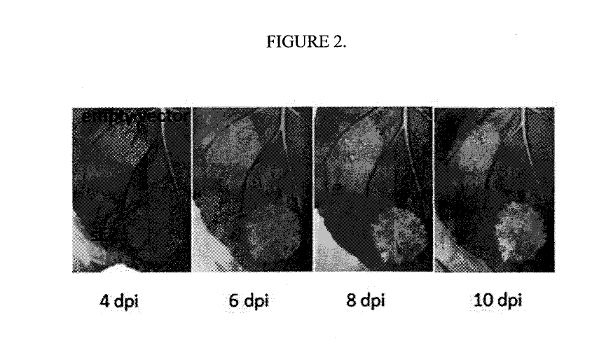

Since no studies about HCV E2 expression in plants have been reported, a time course study was done to examine the expression of the soluble form of HCV E2 (sE2) in N. benthamiana leaves. The germiniviral vector pBYRsE2-711H containing the sE2 coding sequence was introduced into Agrobacterium GV3101 which was later infiltrated into 6 weeks old benthamina leaves at an concentration of OD₆₀₀ 0.2. The procedure of infiltration was previously described (32). An empty germiniviral vector without E2 DNA called BYR1 was treated at the same way and was used as a negative control. The germiniviral vector contains the viral Rep protein (C1/C2 gene) cassette which is required for viral replicon amplification (35). The sE2 expression cassette, driven by the dual-enhancer CaMV 35S promoter, is inserted between the long intergenic region (LIR) and the short intergenic region (SIR) in the viral-sense orientation, replacing the viral movement and coat protein genes. When delivered into plant host, the viral vector can self-splice and become a viral replicon to highly express sE2 protein. Three plants were included in the experiment to average the variability effects from plants. The expression of sE2 was monitored until 20 days post-infiltration (dpi). Leaf samples were harvested at 4, 8, 10, and 12 dpi and used for protein analysis. As shown in FIG. 2, it was observed that necrosis occurred in N. benthamiana leaves after 3 dpi and become pretty strong from day 4 or 5, depending on the growth condition of plants. This indicated that expression of the soluble form of HCV E2 was very toxic to plant leaves and also indicated that the leaf samples should be harvested at an early time before global protein degradation occurred.

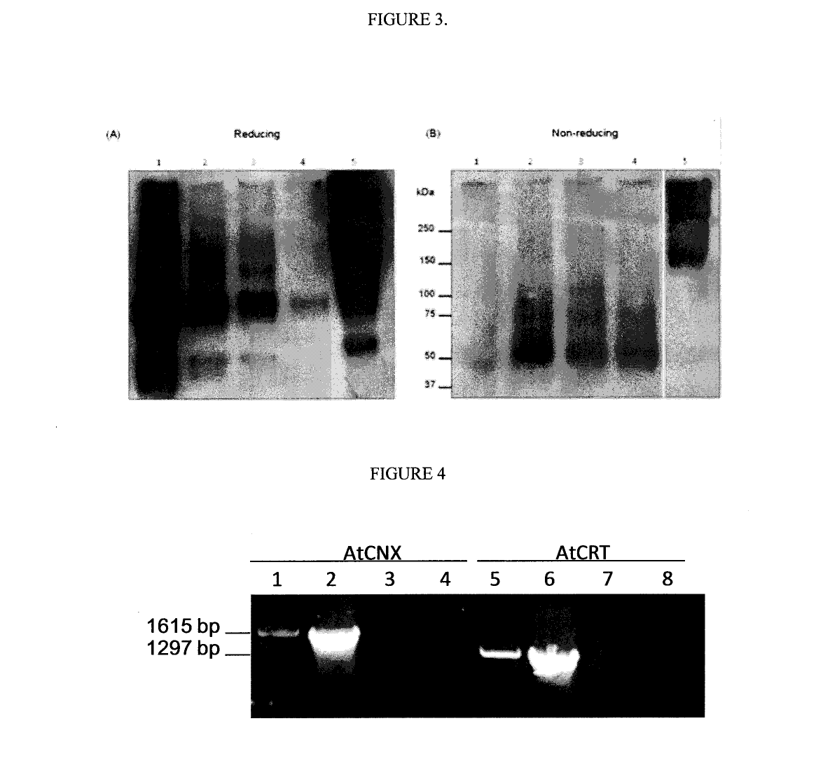

To determine the amount and confirmation of plant-derived sE2, the method of Western blot was used to detect denatured sE2 and conformational sE2 respectively. Total soluble proteins were extracted from leaf samples at day 4, 8, 10 and 12 after infiltration and used 15 μg of total soluble proteins from each sample for Western blot analysis. Correctly folded sE2 can be detected from total soluble protein samples by a conformation-sensitive mouse anti-E2 antibody (anti-conformational E2 antibody). The total amount of sE2 in the samples, including those unfolded and misfolded ones, can be determined by a mouse antibody targeting a linear epitope on E2 (anti-linear E2 antibody), but this required the protein samples to be denatured by DTT and boiling in order to expose the linear epitope. The results of Western blot for denatured sE2 and conformational sE2 are shown in FIG. 3. On the blot for detecting denatured sE2, a decrease of sE2 signal over time could be seen, indicating that protein degradation occurred at sometime between 4 and 8 dpi. The predicted weight of monomeric sE2 is about 50 kDa, but a large portion of high-molecular-weight aggregates could be seen at 4 dpi, which suggested low quality of sE2 production. Those high-molecular-weight aggregates seemed to be not as stable as sE2 dimers and trimers because they degraded faster than the dimers and trimers. In contrast, on the blot for detecting conformational sE2, an increase of sE2 signal was seen at 8, 10, and 12 dpi compared to that at 4 dpi, although the total sE2 signal intensity was weaker than that of denatured sE2. This result firstly showed that only a small portion of sE2 produced in leaves were folded into their correct structures. In other words, the folding efficiency of sE2 is low. Secondly, the result indicated that sE2 folding was slow in the ER because the sE2 signal at 4 dpi was still rather weak. More correctly folded sE2 could be obtained from day 8 samples after infiltration, with a price of reducing the total yield of sE2 due to protein degradation. In all, plant expressed sE2 folded slowly and poorly in the ER; they tended to form aggregates, resulting in degradation of large portions of sE2 produced in leaf cells.

Increased Expression of HCV E2 with the Help of Arabidopsis calreticulin and calnexin

Expression of Arabidopsis calreticulin and calnexin in N. benthamiana. In order to express Arabidopsis calreticulin and calnexin in N. benthamiana, the coding sequences of the AtCRT and AtCNX were released from constructs pBYR-AtCRT and pBYR-AtCNX by digestive enzymes respectively, and then inserted into the non-replicating binary vector pPS1 between corresponding digestion sites respectively, driven by the dual-enhancer CaMV 35S promoter. The vspB 3′ UTR element was replaced by extensin 3′ UTR to improve the functions of 3′ UTR. The expressions of AtCRT and AtCNX in leaves were meaured by reverse-transcription PCR (RT-PCR) using primers AtCRT-Xba-F and AtCRT-Kpn-R for AtCRT, and primers AtCNX-Xba-F and AtCNX-Kpn-R for AtCNX. RNA was extracted from 100 mg leaves expressing calreticulin or calnexin 48 h after agroinfiltration. The procedures of RNA extraction and RT-PCR were previously described above. RNA extraction and purification from each sample were performed in the same way at the same time. The RT-PCR products were observed on agarose gels by electrophoresis (FIG. 4). The electrophoresis result showed that AtCRT and AtCNX were successfully expressed in N. benthamiana leaves, and their expression did not cause necrosis of plant leaves (data not shown).

Co-Expression of the Soluble Form of HCV E2 with Arabidopsis Calreticulin.

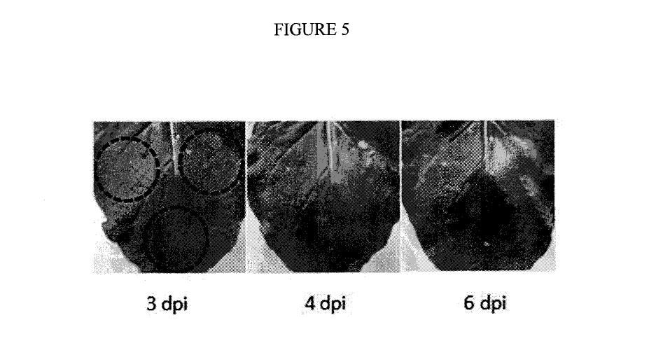

The calreticulin construct and the sE2 construct were co-infiltrated into 6 to 7 weeks old leaves at 1:1 ratio to study the effect of calreticulin on sE2 production and structure. Two types of controls were also infiltrated on the same leaves; one was an empty vector pPS1 and the other was sE2 construct plus pPS1 vector for expression of sE2 alone. The final OD₆₀₀ value of Agrobacterium was 0.2 for all the three treatments, which means for those Agrobacterium with mixed constructs, the OD₆₀₀ value for each construct was 0.1. The expression pattern of sE2 was monitored on leaves of three plants for 8 days after infiltration. It was noticed that with calreticulin treatment, sE2 expression caused even stronger necrosis of leaf cells than sE2 expression alone (FIG. 5). The necrosis began at 3 dpi and developed very quickly on the following day. At day 6 after infiltration, the whole infiltrated area turned yellow and was pretty dried, whereas the leaf spot expressing sE2 alone had much fewer yellow spots in the infiltrated area. The negative control spots infiltrated with pPS1 did not show any necrosis.

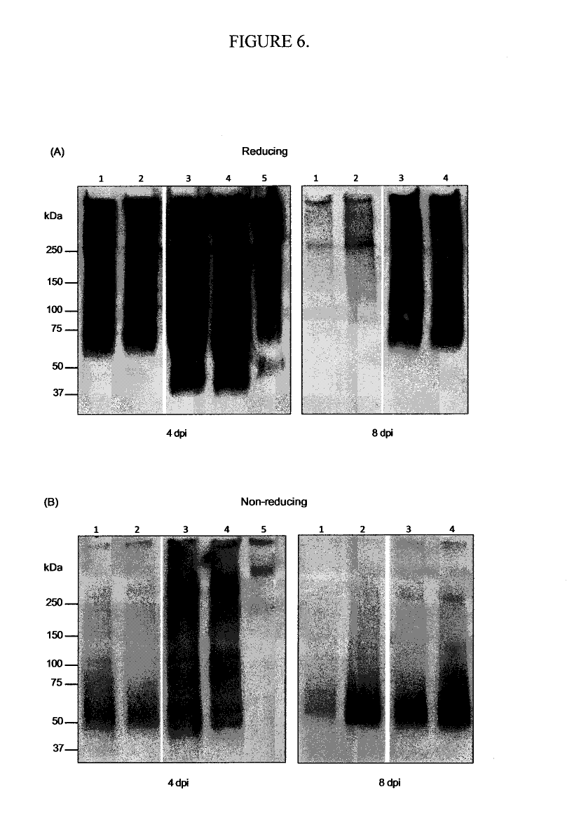

sE2 and sE2/calreticulin leaf samples were harvested at 4 dpi and 8 dpi for protein analysis. 15 μg of total soluble proteins extracted from each leaf sample were added in the SDS-PAGE sample buffer either with 150 mM DTT reducing reagent or without it. For analysis of total sE2 level, the reduced protein samples were further boiled for 10 min so that they could be linearized and recognized by the anti-linear E2 antibodies in the Western blot analysis. On the other hand, to analyze the conformation of sE2, the non-reduced protein samples were directly used in the Western blot to be recognized by the anti-conformational E2 antibodies. The result of the reducing Western blot (FIG. 6A) showed that sE2/calreticulin co-expressing samples produced higher amount of sE2 than sE2/pPS1 samples at both day 4 and day 8 after infiltration. Also, in a comparison of day 4 to day 8 samples, the degree of protein degradation was less in sE2/calreticulin samples than that in sE2/pPS1 samples. These results indicated that calreticulin played a role in preventing protein degradation so that more sE2 could be accumulated in leaves. On the non-reducing Western blot (FIG. 6B), a higher amount of correctly folded sE2 was observed in sE2/calreticulin samples compared to that in sE2/pPS1 samples at day 4 and day 8, with a higher amount in day 8 samples than in day 4 samples. Some high molecular weight bands could also be seen suggesting there were polymers of sE2 in day 4 samples, but they were gone in day 8 samples. This may be because some sE2 was not fully folded at 4 dpi so their hydrophobic regions could still interact with others, although they were in the correct folding track and already formed the conformational epitope, which could be detected by the anti-conformational E2 antibody. In summation, calreticulin greatly increased the yield of sE2 in plant leaves from an early time point and efficiently suppressed protein degradation which was normally observed in sE2 expression. It also helped accumulation of more correct forms of sE2 at an early time point, suggesting its role in facilitating protein folding.

Co-Expression of the Membrane Bound HCV E2 with Arabidopsis Calnexin.

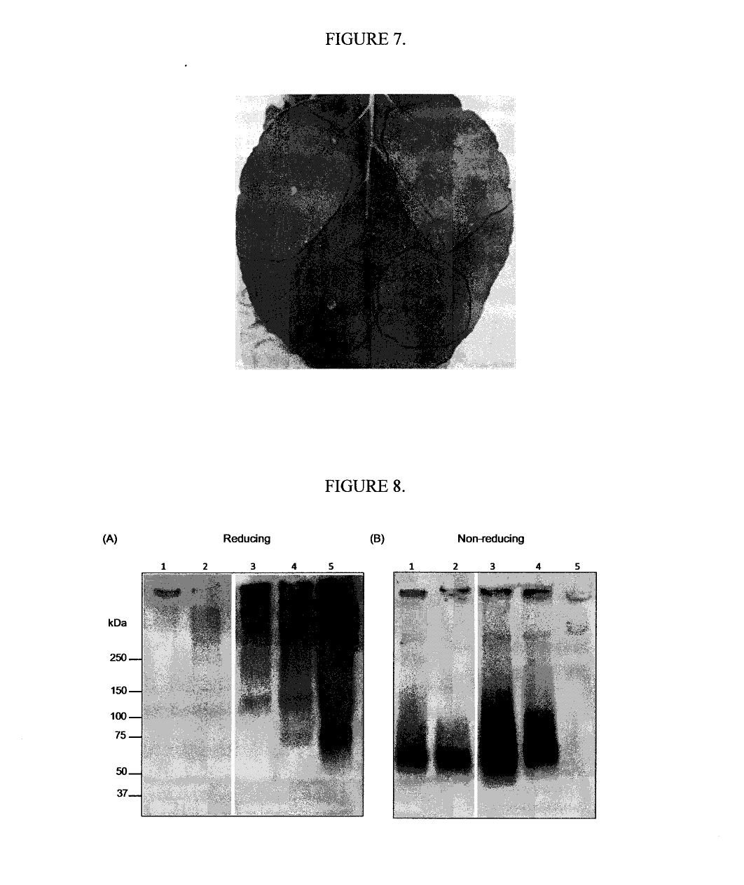

The construct pBYRE2TR expressing a membrane bound HCV E2 (mE2) protein was co-infiltrated with the calnexin construct or the empty binary vector pPS1 into 6 to 7 weeks old leaves at 1:1 ratio to study the effect of calnexin on mE2 production and structure. The empty vector pPS1 and the calnexin construct were also respectively infiltrated into the same leaves as negative controls. The final OD₆₀₀ value of Agrobacterium to be infiltrated was 0.2 for all the treatments. Leaves were monitored for 5 days and harvested at day 5 after infiltration. Co-expression of mE2 and calnexin showed necrosis in leaves at 4 dpi and it became much stronger at 5 dpi (FIG. 7). In addition, mE2/calnexin leaf spots have stronger necrosis than mE2/pPS1 leaf spots. Expression of calnexin alone in leaves showed very little necrotic Proteins were freshly extracted from mE2/calnexin samples and mE2/pPS1 samples harvested at 5 dpi. Since mE2 is a membrane protein, the amount of Triton X-100 was increased to 1% in the extraction buffer to release more membrane proteins into the supernatant of the extracts. The level of denatured mE2 and conformational mE2 were compared between mE2/pPS1 samples and mE2/calnexin samples by Western blot. Both the supernatant and the pellet of protein extract for each sample were tested in the analysis in order to examine all the mE2 proteins produced in leaf samples. The reducing blot showed that a large portion of mE2 produced in mE2/calnexin samples were in the pellet, and the total mE2 signal in mE2/calnexin samples was much stronger than that of mE2/pPS1 samples (FIG. 8A). It appeared the mE2 produced in mE2/pPS1 could not be well recognized by the anti-linear E2 antibody because both the supernatant and the pellet had rather weak mE2 signals. However, the same mE2 protein co-expressed with calnexin had strong mE2 signals. It appeared that the linear epitopes on mE2 proteins were somehow destroyed. On the non-reducing blot shown in FIG. 8B, conformational mE2 signals were observed in mE2/pPS1 samples, indicating that mE2 were expressed in those samples and the correct form of mE2 could be recognized by the anti-conformational E2 antibody. Also, the mE2 signals that could be seen in mE2/calnexin samples were significantly stronger than those in mE2/pPS1 and mE2 proteins were not aggregated. This means that calnexin could help accumulating more correctly folded mE2 in plants; it enhanced the ER's ability on mE2 folding and increased the efficiency of mE2 production.

Co-Expression of the Membrane Bound HCV E2 with Arabidopsis Calnexin and calreticulin.

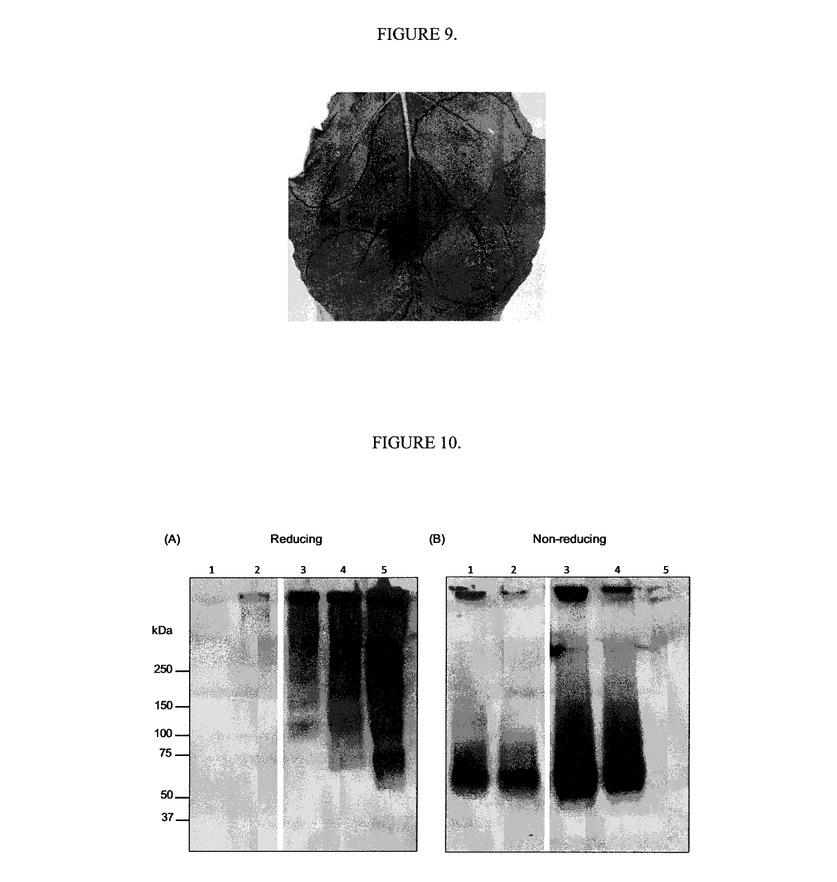

As experiments described herein showed that calreticulin could increase the amount of total sE2 and correctly folded sE2, it was investigated whether calreticulin could coordinate with calnexin to further facilitate membrane bound E2 folding and production. Therefore, the constructs expressing mE2, calnexin and calreticulin were co-infiltrated into 6-7 weeks old leaves at 1:1:1 ratio to test the effect of combined calnexin and calreticulin treatment on mE2 expression. mE2 alone was expressed in a different spot on same leaves by co-infiltration of mE2 construct and pPS1 at 1:2 ratio. Besides, leaf spots infiltrated with pPS1 and co-infiltrated with calnexin and calreticulin were used as controls. The total OD₆₀₀ of Agrobacterium for infiltration was 0.3 per treatment, and leaves of 3 different plants were infiltrated. Infiltrated leaves were monitored for 5 days and phenotype changes were Necrosis occurred at 3 dpi in all the leaf spots expressing transgenes, and at 5 dpi the leaf spots co-expressing mE2, calnexin, calreticulin were much more necrotic than those only expressing mE2 (FIG. 9). Expression of calnexin and calreticulin without mE2 also caused a little necrosis, compared to the pPS1 negative control.

mE2/calnexin/calreticulin samples and mE2/pPS1 samples were harvest at 5 dpi for mE2 protein analysis by Western blot. 1% Triton X-100 was added to the extraction buffer to destroy the membrane structures in cells and included both the supernatant and pellet of each leaf crude extract in the analysis as was done in the mE2/calnexin co-expression experiment. The reducing Western blot analysis again showed no or very low mE2 signal in mE2/pPS1 samples, indicating that the linear epitope of E2 was lost. Nevertheless, strong mE2 signals were observed in the supernatant and pellet of mE2/calnexin/calreticulin samples (FIG. 10A). The pellet contained even more monomeric mE2 than the supernatant. The non-reducing Western blot (FIG. 10B) showed that correctly folded mE2 were accumulated in both mE2/pPS1 samples and mE2/calnexin/calreticulin samples, but the level of correct form of mE2 was significantly higher in mE2/calnexin/calreticulin samples than in mE2/pPS1 samples. This indicated that a combination of calnexin and calreticulin treatment could also effectively increase the level of properly folded mE2. However, compared to FIG. 7, it appeared that mE2 expression pattern was very similar between calnexin treatment and calnexin/calreticulin treatment. No better effect was observed when mE2 was co-expressed with calnexin and calreticulin together than mE2 expressed with calnexin alone.

Quality Improvement of Plant Produced HCV E2 with the Help of bZIP60 and bZIP60ΔC

Over-Expression of bZIP60 and bZIP60ΔC in N. benthamiana Leaves.

The coding sequence of NbbZIP60 and NbbZIP60ΔC were amplified by high-fetidity PCR from total cDNA of N. benthamiana wild type plant leaves. The PCR products were respectively inserted into a binary vector between Nos promoter and Nos 3′ UTR, so that NbbZIP60 and NbbZIP60ΔC could be constitutively expressed when they were transformed into N. benthamiana leaves. The coding sequences of AtbZIP60 and AtbZIP60ΔC (amino acid 1-216) are shown in FIGS. 1D and 1E, respectively. The cDNA fragments of AtbZIP60 and AtbZIP60ΔC were respectively inserted into the non-replicating binary vector pPS1, between the dual-enhancer CaMV 35S promoter and vspB 3′ UTR. Driven by the strong 35S promoter, the resulting constructs psAtbZIP60 and psAtbZIP60-S216 could highly express AtbZIP60 and AtbZIP60ΔC respectively in N. benthamiana leaves.

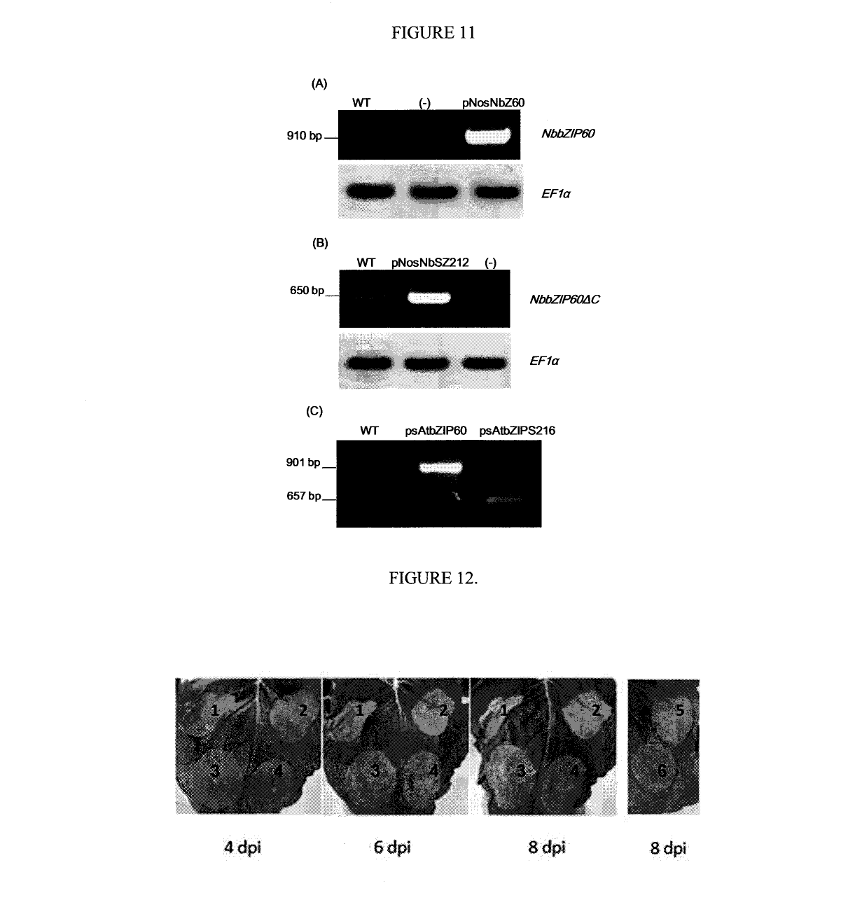

The four constructs were introduced into N. benthamiana leaves via Agrobacterium to examine whether they cause necrosis of plants. The phenotypes of leaves were checked for 1 week and the leaves stayed normal. Therefore, transient expression of bZIP60 or bZIP60ΔC did not cause toxic effects to plants (data not shown). At 2 dpi, leaf samples were harvested for RT-PCR in order to confirm gene overexpression. For each construct, 3 different leaf samples were used as replicates. Wild type leaves were used as controls. RNA extraction and purification from each sample were performed in the same way at the same time. Same amount of total RNA from each sample was used in RT-PCR and the procedure was previously described above. The RT-PCR results showed that NbbZIP60 and NbbZIP60ΔC were overexpressed in leaves because their band signals were much higher than those from wild type samples representing the endogenous NbbZIP60 and NbbZIP60ΔC mRNA levels (FIG. 11A, B). The RT-PCR result shown in FIG. 11C also indicated that AtbZIP60 and AtbZIP60ΔC were highly expressed in N. benthamiana leaves.

Co-Expression of the Soluble Form of HCV E2 with bZIP60 or bZIP60ΔC.

The construct expressing soluble form of HCV E2 (sE2) was co-infiltrated with NbbZIP60 construct, NbbZIP60ΔC construct, AtbZIP60 construct and AtbZIP60ΔC construct respectively into 6-7 weeks old N. benthamiana leaves in order to examine whether bZIP60 and bZIP60ΔC could promote HCV E2 folding. sE2 construct plus empty vector pPS1 were co-infiltrated into the leaves to express sE2 alone for comparison. Agrobacterium carrying different constructs were mixed at 1:1 ratio, and the total OD₆₀₀ for infiltration was 0.2. FIG. 12 showed phenotypes of leaf spots expressing sE2, sE2/NbbZIP60, sE2/NbbZIP60ΔC and sE2/AtbZIP60ΔC at 4, 6, and 8 dpi and leaf spot expressing sE2/AtbZIP60 at 8 dpi. sE2/NbbZIP60 treated leaf spot showed stronger necrosis than sE2 leaf spot and other treated leaf spots. However, sE2/NbbZIP60ΔC and sE2/AtbZIP60ΔC treated leaf spots had similar degrees of necrosis to that of sE2 leaf spot. sE2/AtbZIP60 was co-infiltrated into different leaves in the same growth condition, and co-expression of sE2/AtbZIP60 also showed similar necrotic effect to the sE2 leaf spot.

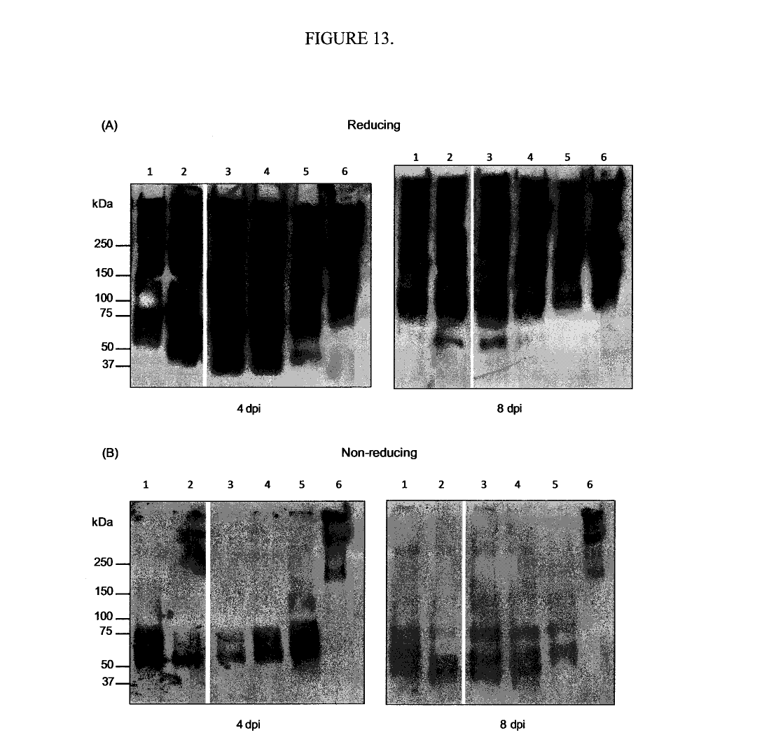

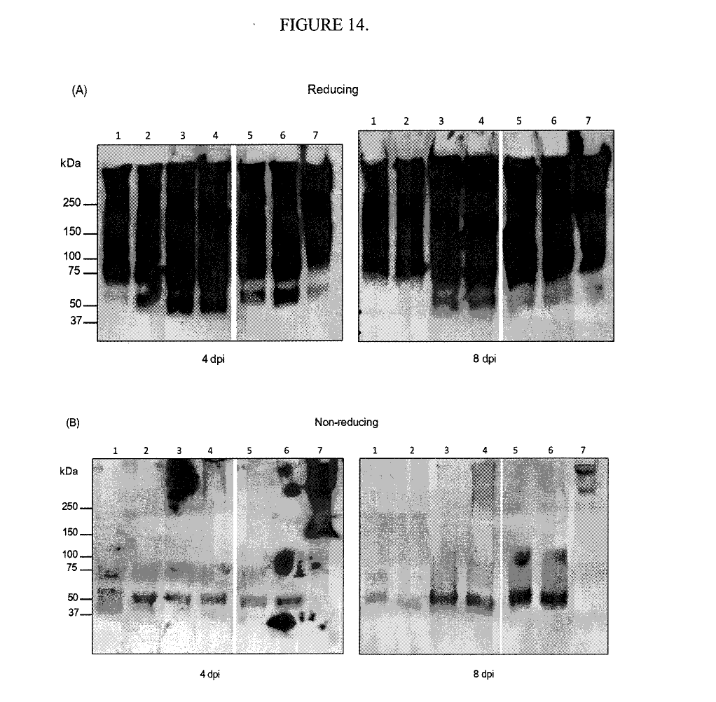

Day 4 and day 8 samples were harvested for analysis of sE2 production with the help of each bZIP60 protein and bZIP60ΔC protein. Reducing and non-reducing Western blots were performed to compare the total sE2 levels and correctly folded sE2 levels between sE2 samples and sE2/bZIP60 or sE2/bZIP60ΔC samples. The results of reducing and non-reducing Western blots were shown in FIG. 13. The reducing blots showed that sE2 alone and sE2 with treatments had similar expression levels of sE2, although sE2/NbbZIP60ΔC, sE2/AtbZIP60, and sE2/AtbZIP60ΔC samples seemed to express more monomeric sE2. A portion of sE2 was degraded in all samples at 8 dpi, and the degradation degrees are similar for all samples. Therefore, any benefit of bZIP60 and bZIP60ΔC was not seen in regards to the yield of sE2. On the other hand, the non-reducing blots showed that at day 4, expression of sE2 with treatments did not increase the level of correct form of sE2. However, in day 8 samples, sE2/NbbZIP60 and sE2/AtbZIP60ΔC samples seemed to have more correctly folded sE2 than sE2 samples, although the benefits were not significant. To ensure NbbZIP60 and AtbZIP60ΔC could help accumulating more properly folded sE2, more sE2/NbbZIP60 and sE2/AtbZIP60ΔC samples were tested by reducing and non-reducing Western blots (FIG. 14). The repeated experiments confirmed that sE2/NbbZIP60 and sE2/AtbZIP60ΔC samples contains more correct form of sE2 proteins than sE2 alone samples at 8 dpi but not 4 dpi, indicating that the helping effects of NbbZIP60 and AtbZIP60ΔC on sE2 production took time. However, compared to the total sE2 level suggested by the reducing Western blots, correct form of sE2 were still very little with NbbZIP60 or AtbZIP60ΔC treatments. Overall, NbbZIP60 and AtbZIP60ΔC did not seem to increase the amount of plant produced sE2, but they helped to improve the quality of sE2 to some extent.

Relationship Between bZIP60 and BIP Expression in N. benthamiana.

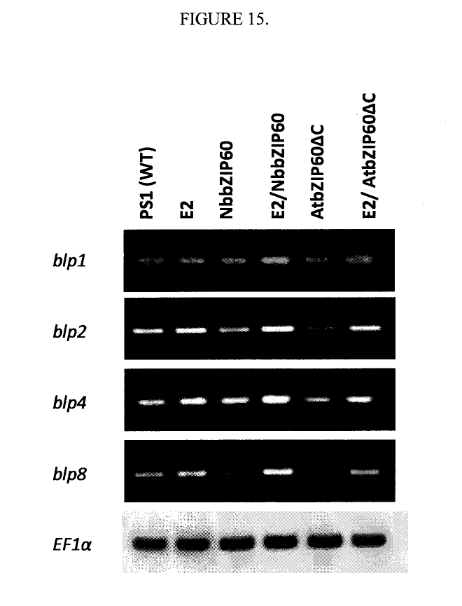

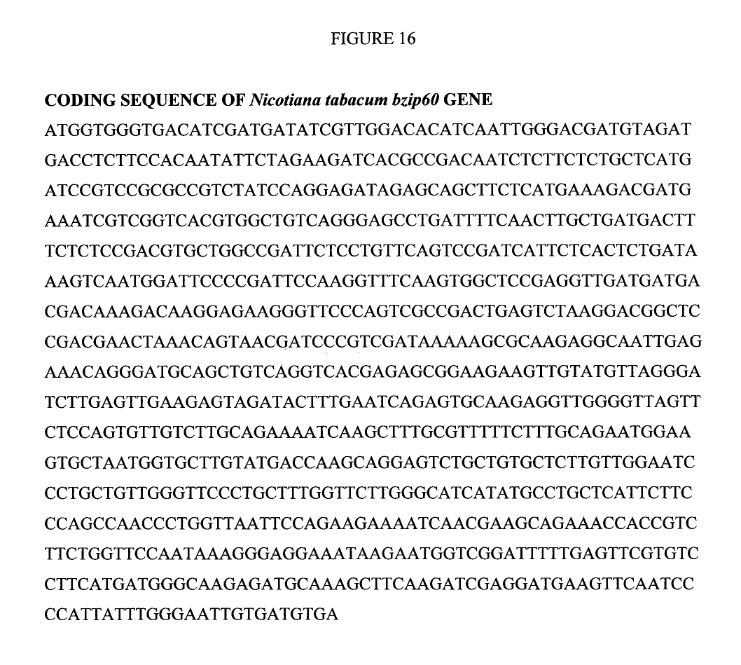

From the observations in the previous experiment, bZIP60 and its putative active form bZIP60ΔC did not significantly promote the folding and production of sE2 in plants. To find out possible reasons, BiP expression levels were examined in leaves infiltrated with sE2/pPS1, sE2/NbbZIP60 or sE2/AtbZIP60ΔC constructs to see if NbbZIP60 or AtbZIP60ΔC treatment under ER stress condition increases the BiP genes expression, since these two treatments showed a little help on sE2 folding. BiP expression levels were also examined in leaves infiltrated with only NbbZIP60 or AtbZIP60ΔC construct to see whether overexpression of NbbZIP60 or NbbZIP60ΔC without ER stress can induce the expression of BiPs. In N. benthamiana, only one BiP cDNA sequence was found in the Genebank which was called luminal binding protein 4 (Blp4) (Genebank accession number FJ463755). But, 5 more cDNA sequences of Blp genes were found in N. tabacum. Since N. benthamiana and N. tabacum are closely relative species, primers were designed based on tobacco Blp sequences and used them to amplify the orthologs of tobacco Blp1 (Genebank accession # X60060.1), Blp2 (X60059.1), Blp4 (X60057.1) and Blp8 (X60062.1) in N. benthamiana. Leaf samples were harvested 48 h after infiltration and total RNA were extracted from them for RT-PCR analysis. Three different leaf samples were tested for each construct to ensure the reliability of the result. Wild type leaves infiltrated with pPS1 were used as controls. RNA extraction and purification from samples were performed at the same time, following the procedures described above. 1 μg total RNA extracted from each sample was used to perform RT-PCR, and cDNA of Blp genes were amplified with their corresponding pairs of primers. A fragment of constitutively expressed EF1α was also amplified from each sample to serve as internal control. The result of Blps expression was observed by electrophoresis of RT-PCR products (FIG. 15). The result showed that N. benthamiana Blp expression levels increased only in response to HCV E2 treatment, but not to NbbZIP60 or AtbZIP60ΔC treatment. In addition, Blp levels were about the same between wild type samples and NbbZIP60 or AtbZIP60ΔC samples, indicating that overexpression of NbbZIP60 or AtbZIP60ΔC in leaves without ER stress did not activate Blp genes expression. Furthermore, among those samples expressing sE2, the expression levels of Blps were also very similar, which suggested that overexpression of NbbZIP60 or AtbZIP60AC under ER stress condition could not induce Blps expression, either. Expression of Blps was increased in response to the ER stress generated by HCV E2 expression, but not induced by NbbZIP60 or AtbZIP60ΔC. This may be the reason why overexpression of bZIP60 and bZIP60ΔC in leaves expressing sE2 did not significantly promote HCV E2 folding and accumulation.

Arabidopsis Calreticulin and Calnexin Promote HCV E2 Protein Production in N. benthamiana

As described herein, Arabidopsis calreticulin and calnexin did help to increase the yield of HCV E2 and also improve the folding quality of HCV E2. It was found that with calreticulin or calnexin treatment, protein degradation was effectively suppressed, which was consistent with other published studies (12. 31). The suppression of protein degradation may be because the unfolded or misfolded proteins were stabilized by calnexin and calreticulin when associated with them, escaping from digestion by proteases. It is also possible that with the help of calreticulin and calnexin, protein folding efficiency was increased so that the ER stress was effectively reduced, weakening the signal that triggered protein degradation in the ER. Also, more HCV E2 proteins were expressing in their correct conformation in calreticulin or calnexin treated samples, especially at early time point. This may be simply because fewer proteins were degraded, or because calreticulin and calnexin directly assisted E2 folding and maturation by recruiting folding factors such as ERp57 to catalyze protein folding (36). This result is encouraging because the goal is to rapidly produce large amounts of functional HCV E2 proteins so as to save time and money when manufacturing this vaccine candidate in the future.

Previous studies also showed that calreticulin and calnexin could efficiently prevent protein aggregation in the ER. However, in the experiments described herein, it is hard to tell whether calreticulin and calnexin prevented protein aggregation. In the Western blot analysis, it seemed the protein aggregates in E2/calreticulin or E2/calnexin samples were even more than those in E2 alone samples. But it should be noted that the total E2 level in calnexin or calreticulin treated samples was also much higher than that in E2 alone samples, so it is difficult to determine the percentage of aggregates in total E2 proteins from Western blot. The quality of the plant produced HCV E2 may be further tested by a CD81 binding assay. The recombinant E2 produced in the plant system will be regarded as functional vaccines if they are able to bind CD81, because CD81 is the putative receptor of HCV E2 on human cells. Otherwise, the immune response induced by E2 vaccination cannot effectively block the entry of HCV into cells.

Overexpression of bZIP60 or bZIP60ΔC has Small Effect on Facilitating HCV E2 Folding in the ER

It was hypothesized that overexpression of the ER stress transducer bZIP60 or its active form bZIP60ΔC in N. benthamiana leaves expressing HCV E2 could up-regulate the expression of a group of UPR genes especially Bip, which could then efficiently assist HCV E2 folding and maturation. However, the experiment showed that Bips (or Blps) expression could not be up-regulated by either NbbZIP60 or AtbZIP60ΔC, no matter whether HCV E2 caused ER stress or not, but their expression was indeed increased in response to HCV E2 expression. As a result, a significant help was not received from overexpression of bZIP60 or bZIP60ΔC for HCV E2 folding and accumulation. Only the truncated form of AtbZIP60 and NbbZIP60 helped a little in expressing more correctly folded E2, but the total yield of correct form of E2 was still relatively low.

From those results, it can be confirmed that HCV E2 induced ER stress creates a response in leaf cells due to the increased expression level of BiP genes, but it seems that BiPs are not activated by the NbbZIP60 pathway. This is possible because there are other bZIP proteins in Arabidopsis that also have the capability of Bip activation, such as bZIP28. It is possible that in N. benthamiana bZIP60 loses the function to activate BiP, and that function is maintained in other bZIP proteins. It is also possible that NbbZIP60 can activate some other BiP genes or UPR genes that are not tested in this experiment. But even if that is the case, those UPR genes do not seem to be effective in facilitating glycoprotein folding, because the protein level of correctly folded E2 in E2/AtbZIP60ΔC and E2/NbbZIP60 co-expressed samples were not increased a lot. Therefore, experiments to test whether NbbZIP60 can activate UPR genes in N. benthamiana may be designed. BiP and other UPR genes which are activated by AtbZIP60 in Arabidopsis contain the ER stress response element (ERSE) or the plant unfolded protein response element (p-UPRE) before their promoters (23). Thus, reporter constructs may be made that have the ERSE or the p-UPRE element inserted before the promoter, which can then be tested to determine if NbbZIP60ΔC can transactivate the expression of reporter genes. If it can, that means NbbZIP60 has the ability to activate UPR genes containing ERSE or p-UPRE elements. Then maybe there are other unidentified Bip genes in N. benthamiana that can be induced by NbbZIP60. If it is unable to, that means NbbZIP60 may not play a role in UPR gene activation in N. benthamiana. Therefore, other methods to up-regulate Bip expression may be tried in order to promote HCV E2 production.

### REFERENCES

1. Deleersnyder, V., Pillez, A., Wychowski, C., Blight, K., Xu, J., Hahn, Y. S., Rice, C. M., Dubuisson, J. (1997). Formation of native hepatitis C virus glycoprotein complexes. Journal of Virology, 71, 697-704.
2. Heile, J. M., Fong, Y. L., Rosa, D., Burger, K., Saletti, G., Campagnoli, S., . . . Abrignani, S. (2000). Evaluation of Hepatitis C Virus Glycoprotein E2 for Vaccine Design: an Endoplasmic Reticulum-Retained Recombinant Protein Is Superior to Secreted Recombinant Protein and DNA-Based Vaccine Candidates. Journal of Virology, 74, 6885-6892.
3. Alberti, A., Chemello, L., Benvegnu, L. (1999). Natural history of hepatitis C. J Hepatol, 31(Suppl. 1), 17-24.
4. Choo, Q. L., Kuo, G., Weiner, A. J., Overby, L. R., Bradley, D. W., Houghton, M. (1989). Isolation of a cDNA clone derived from a blood-borne non-A, non-B viral hepatitis genome. Science, 244, 359-362.
5. Rosa, D., Campagnoli, S., Moretto, C., Guenzi, E. H., Cousens, L., Chin, M. Dong, C. Weiner, A. J., Lau, J. Y. N., Choo, Q. L., Chien, D., Pileri, P., Houghton, M., Abrignani, S. (1996). A quantitative test to estimate neutralizing antibodies to the hepatitis C virus: cytofluorimetric assessment of envelope glycoprotein 2 binding to target cells. Proc. Natl. Acad. Sci. USA, 93, 1759-1763.
6. Pileri, P., Uematsu, Y., Campagnoli, S., Galli, G., Falugi, F., Petracca, R., Weiner, A. J., Houghton, M., Rosa, D., Grandi, G., Abrignani, S. (1998). Binding of hepatitis C virus to CD81. Science, 282, 938-941.
7. Van den Berg, B., Ellis, R. J., Dobson, C. M. (1999). Effects of macromolecular crowding on protein folding and aggregation. EMBO J, 18, 6927-6933.
8. Agashe, V. R., Hartl, F. U. (2000). Roles of molecular chaperones in cytoplasmic protein folding. Seminars in Cell & Developmental Biology, 11, 15-25. doi:10.1006.
9. Michalak, M., Milner, R. E., Burns, K. Opas, M. (1992). Calreticulin. Biochem. J., 285, 681-692.
10. Bergeron, J. J., Brenner, M. B., Thomas, D. Y. Williams, D. B. (1994). Calnexin: a membrane-bound chaperone of the endoplasmic reticulum. Trends Biochem Sci., 19, 124-128.
11. Hebert, D. N., Foellmer, B. Helenius, A. (1995). Glucose trimming and reglucosylation determine glycoprotein association with calnexin in the endoplasmic reticulum. Cell, 81, 425-433.
12. Hebert, D. N., Foellmer, B., Helenius, A. (1996). Calnexin and calreticulin promote folding, delay oligomerization and suppress degradation of influenza hemagglutinin in microsomes. EMBOJ., 15, 2961-8.
13. Peterson, J. R., Ora, A., Van, P. N., Helenius, A. (1995). Transient, Lectin-like Association of Calreticulin with Folding Intermediates of Cellular and Viral Glycoproteins. Mol Biol Cell., 6, 1173-1184.
14. Mayer, M., Kies, U., Kammermeier, R., Buchner, J. (2000). BiP and PDI cooperate in the oxidative folding of antibodies in vitro. J. Biol. Chem. 275, 29421-29425.
15. Stronge, V S., Saito, Y., Ihara, Y., Williams, D. B. (2001). Relationship between calnexin and BiP in suppressing aggregation and promoting refolding of protein and glycoprotein substrates. J Biol Chem., 276, 39779-39787.
16. Ron, D., Walter, P. (2007). Signal integration in the endoplasmic reticulum unfolded protein response. Nat Rev Mol Cell Biol., 8, 519-529.
17. Mori, K., Kawahara, T., Yoshida, H., Yanagi, H., Yura, T. (1996). Signalling from the endoplasmic reticulum to the nucleus: transcription factor with a basic-leucine zipper motif is required for the unfolded protein-response pathway. Genes Cells, 1, 803-817.
18. Schröder, M. and Kaufman, R. J. (2005) The mammalian unfolded protein response. Annu. Rev. Biochem. 74, 739-789.
19. Pavio, N., Romano, P. R., Graczyk, T. M., Feinstone, S. M., Taylor, D. R. (2003). Protein synthesis and endoplasmic reticulum stress can be modulated by the hepatitis C virus envelope protein E2 through the eukaryotic initiation factor 2alpha kinase PERK. Journal of Virology, 77, 3578-3585.
20. Yoshida, H., Okada, T., Haze, K., Yanagi, H., Yura, T., Negishi, M., Mori, K. (2000).

ATF6 activated by proteolysis binds in the presence of NF-Y [CBF] directly to the cis-acting element responsible for the mammalian unfolded protein response. Mol. Cell Biol., 20, 6755-6767.
21. Tardif, K. D., Mori, K., Kaufman, R. J., Siddiqui, A. (2004). Hepatitis C virus suppresses the IRE1-XBP1 pathway of the unfolded protein response. J Biol Chem., 279, 17158-17164.
22. Urade, R. (2009). The endoplasmic reticulum stress signaling pathways in plants. Biofactors, 35, 326-331. Review.
23. Iwata, Y., Fedoroff, N. V., Koizumi, N. (2008). Arabidopsis bZIP60 Is a Proteolysis-Activated Transcription Factor Involved in the Endoplasmic Reticulum Stress Response. The Plant Cell, 20, 3107-3121.
24. Liu, J. X., Srivastava, R., Che, P., Howell, S. H. (2007). An endoplasmic reticulum stress response in Arabidopsis is mediated by proteolytic processing and nuclear relocation of a membrane-associated transcription factor, bZIP28. Plant Cell, 19, 4111-4119.
25. Iwata, Y., Fedoroff, N. V., Koizumi, N. (2009). The Arabidopsis membrane-bound transcription factor AtbZIP60 is a novel plant-specific endoplasmic reticulum stress transducer. Plant Signal Behavior, 4, 514-516. doi: 10.1105.
26. Tateda, C., Ozaki, R., Onodera, Y., Takahashi, Y., Yamaguchi, K., Berberich, T., Koizumi, N., Kusano, T. (2008). NtbZIP60, an endoplasmic reticulum-localized transcription factor, plays a role in the defense response against bacterial pathogens in Nicotiana tabacum. J Plant Res., 121, 603-611.
27. Grakoui, A., Wychowski, C., Lin, C., Feinstone, S. M., Rice, C. M. (1993). Expression and identification of hepatitis C virus polyprotein cleavage products. Journal of Virology, 67, 1385-1395.
28. Dubuisson, J., Hsu, H. H., Cheung, R. C., Greenberg, H. B., Russell, D. G., Rice, C. M. (1994). Formation and intracellular localization of hepatitis C virus envelope glycoprotein complexes expressed by recombinant vaccinia and Sindbis viruses. Journal of Virology, 68, 6147-6160.
29. Choukhi, A., Ung, S., Wychowski, C., Dubuisson, J. (1998). Involvement of endoplasmic reticulum chaperones in the folding of hepatitis C virus glycoproteins. Journal of Virology, 72, 3851-3858.
30. Iwata, Y., Koizumi, N. (2005). An Arabidopsis transcription factor, AtbZIP60, regulates the endoplasmic reticulum stress response in a manner unique to plants. Proc Natl Acad Sci USA., 102, 5280-5285.
31. Hebert, D. N., Zhang, J. X., Chen, W., Foellmer, B., Helenius, A. (1997). The number and location of glycans on influenza hemagglutinin determine folding and association with calnexin and calreticulin. J Cell Biol., 139, 613-623.
32. Huang, Z., Chen, Q., Hjelm, B., Arntzen, C., Mason, H. (2009). A DNA replicon system for rapid high-level production of virus-like particles in plants. Biotechnol Bioeng., 103, 706-714.
33. Judge, N. A., Mason, H. S., O'Brien, A. D. (2004). Plant cell-based intimin vaccine given orally to mice primed with intimin reduces time of Escherichia coli O157:H7 shedding in feces. Infect. Immun. 72, 168-175.
34. Becker, D., Kemper, E., Schell, J., Masterson, R. (1992). New plant binary vectors with selectable markers located proximal to the left T-DNA border. Plant Mol Biol. 20, 1195-1197.
35. Laufs, J., Jupin, I., David, C., Schumacher, S., Heyraud-Nitschke, F., Gronenborn. B. (1995). Geminivirus replication: genetic and biochemical characterization of Rep protein function, a review. Biochimie, 77, 765-773.
36. High, S., Lecomte, F. J., Russell, S. J., Abell, B. M., Oliver, J. D. (2000) Glycoprotein folding in the endoplasmic reticulum: a tale of three chaperones? FEBS Lett., 476, 38-41. Review.

TABLE 1
Primers Used In Example 1

| Name | Sequence |
|---|---|
| NbbZIP60- | 5′ TGGCATGGTGGGTGACATCGATGATATC 3′ |
| Nco-F | |
| NbbZIP60- | 5′ CCGAGCTCTCACATCACAATTCCCAAATA 3′ |
| Sac-R | |
| NbbZIP60- | 5′ CCGAGCTCTCAAGACTCCTGCTTGGTCAT 3′ |
| S212 | |
| pUni51-F | 5′ CGGGTACCTCAAGACTCCTGCTTCGACATC 3′ |
| AtbZIP60- | 5′ GCGGTACCCGTTGTCACGCCG 3′ |
| Kpn-R | |
| AtbZIP60- | 5′ CGGGTACCTCAAGACTCCTGC 3′ |
| S216-K | |
| NtB1p1-F | 5′ GCTGCTGTTCAAGGTGGTA 3′ |
| NtB1p1-R | 5′ TGGTTGGGATGACGGTGTT 3′ |
| NtB1p2-F | 5′ GCAACCCAATTATCACAGC 3′ |
| NtB1p2-R | 5′ GTAACCCTCACCTCAACCT 3′ |
| NtB1p4-F | 5′ ACGGAAAGGACATCAGCAAG 3′ |
| NtB1p4-R | 5′ GTGCCCGAGTAAGTGGTTCA 3′ |
| NtB1p8-F | 5′ GCAACCCAATTATCACAGC 3′ |
| NtB1p8-R | 5′ GTAACCCTCACCTCAACCT 3′ |
| AtCRT-Xba-F | 5′ CCTCTAGAACAATGGCGAAAC 3′ |
| AtCRT-Kpn-R | 5′ GGGGTACCTTAAAGCTCGTCA 3′ |
| AtCNX-Xba-F | 5′ CCTCTAGAACAATGAGACAAC 3′ |
| AtCNX-Kpn-R | 5′ GGGGTACCTTGTTCTAATTAT 3′ |
| EF1α-F | 5′ CTGGTGGTTTTGAAGCTGGTA 3′ |
| EF1α-R | 5′ GGTGGTAGCATCCATCTTGTT 3′ |

### Example 2

### Ebola Virus Glycoprotein Expression Enhanced by Co-Expression of Calreticulin

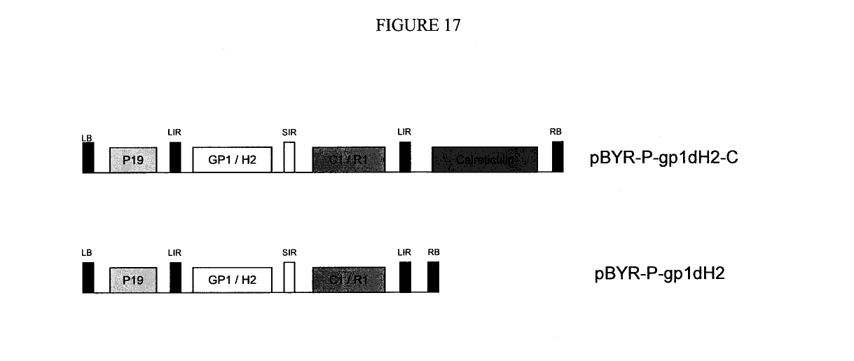

Ebola virus causes a highly mortal hemorrhagic fever in humans and is considered a bioterror threat agent. The Ebola virus glycoprotein (GP1) was expressed as a fusion with the heavy chain of anti-GP1 monoclonal antibody 6D8 (Phoolcharoen et al., (2011) Plant Biotechnol. J., 9(7):807-16; Phoolcharoen et al., (2011) Proc. Natl. Acad. Sci. USA 108(51):20695-20700). It was noted that expression of the GP1 fusion protein caused substantial necrosis in leaves of N. benthamiana, and it was hypothesized that ER stress due to slow GP1 protein folding was the cause. Thus, experiments were conducted to determine whether over-expression of ER chaperone calreticulin with GP1-H2 could enhance expression. Two expression vectors were constructed (FIG. 17) that contain a geminiviral replicon for expression of the GP1-heavy chain fusion (GP1-H2). pBYR-P-gp1dH2 also contains an expression cassette for p19, a gene silencing inhibitor. pBYR-P-gp1dH2-C contains the p19 cassette and another cassette for expression of A. thaliana calreticulin (CRT).

For expression testing, leaves of N. benthamiana plants were inoculated with Agrobacterium tumefaciens GV3101 carrying the T-DNA plasmids shown in FIG. 17, essentially as described (Phoolcharoen et al., (2011) Plant Biotechnol. J., 9(7):807-16). The two constructs, identical except for the presence or absence of the CRT expression cassette, were inoculated on opposite sides of the same leaves, so that direct comparisons of the constructs could be made in the same leaves. Four days after agroinoculation, leaf samples were harvested and extracted in nondenaturing buffer (phosphate-buffered saline, 50 mM sodium ascorbate, 1 mM EDTA, 10 μg/ml leupeptin, 0.1% Triton X-100), using homogenization with a bead-beater. The samples were centrifuged at 5,000 g at 4° C. for 10 min, and the supernatants collected and treated with an equal volume of 2×SDS sample buffer (no reducing agent). The pellets were resuspended in an equal volume of 1×SDS sample buffer (no reducing agent). Samples in 1×SDS sample buffer were resolved by electrophoresis in 4-12% polyacrylamide gradient gels, and proteins electro-transferred to PVDF membrane for Western blot probing.

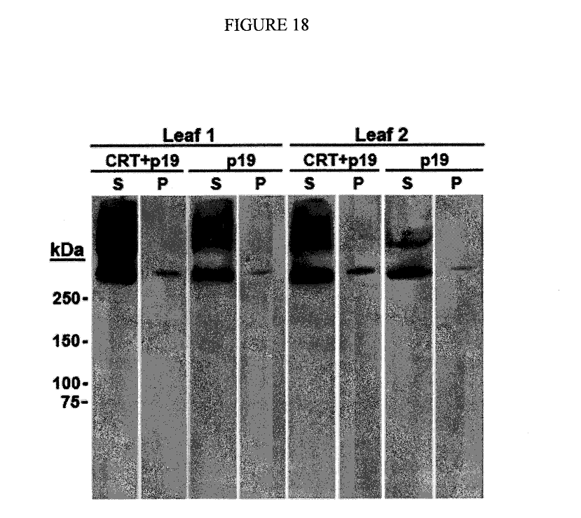

In order to assess correct folding of GP1, the blot was probed with a conformation-dependent anti-GP1 mouse monoclonal antibody 13C6 (Phoolcharoen et al., (2011) Plant Biotechnol. J., 9(7):807-16). FIG. 18 shows the Western blots with samples obtained from two different leaves. It was observed that in samples from both leaves, the co-expression of CRT resulted in a higher level of GP1 signal than without CRT. The great majority of GP1-H2 protein was in the soluble fraction (S) whether or not CRT was co-expressed. Thus, it was concluded that CRT co-expression enhanced accumulation of GP1-H2 fusion protein in leaves.

### Example 3

### Over-Expression of Nicotiana benthamiana bZIP60 Transcription Factor for Upregulation of ER Chaperones and Enhanced Expression of Recombinant Proteins Targeted to the ER

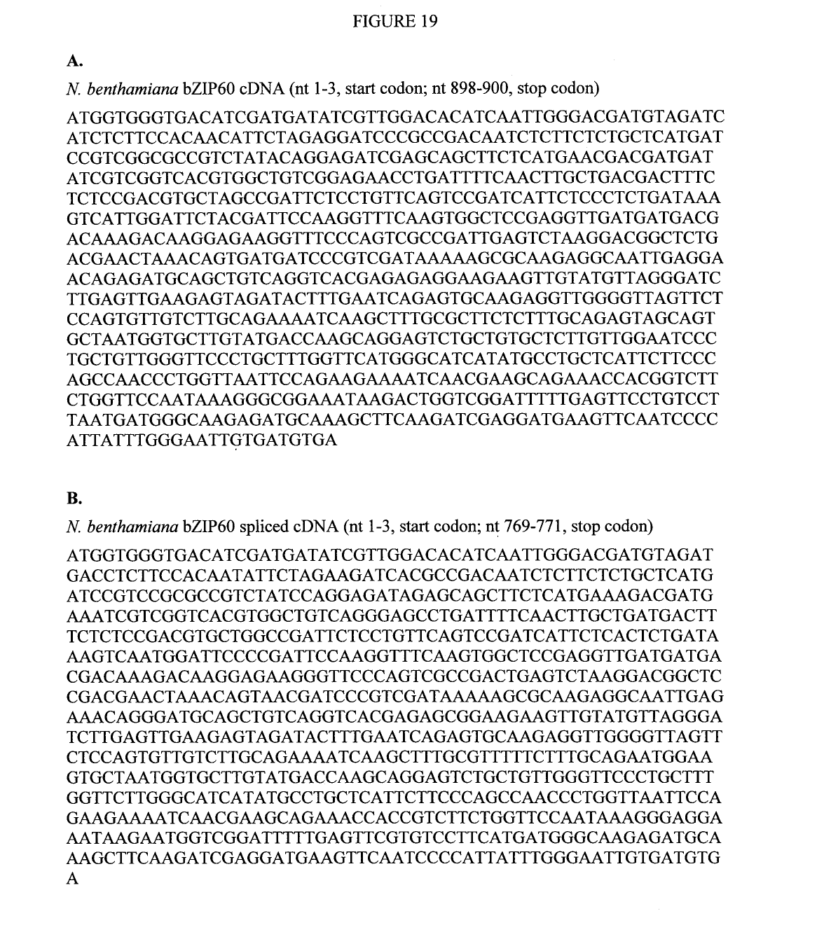

The N. benthamiana bZIP60 cDNA was cloned as described in Example 1. The nucleotide sequence of this cDNA is shown in FIG. 19A. The theory at the time postulated that a membrane-anchored bZIP60 was released from the membrane by proteolysis under certain conditions, and the released N-terminal fragment was transported to the nucleus to upregulate ER chaperones. In 2011, new research showed (Deng et al., (2011) Proc Natl Acad Sci USA. 2011 Apr. 26; 108(17):7247-52) that in Arabidopsis thaliana, heat stress induces the cytoplasmic splicing of bZIP60 mRNA to create a coding sequence that includes a C-terminal nuclear targeting signal. Transport of the protein product of the spliced bZIP60 mRNA into the nucleus results in upregulation of genes related to ER stress, including chaperones.

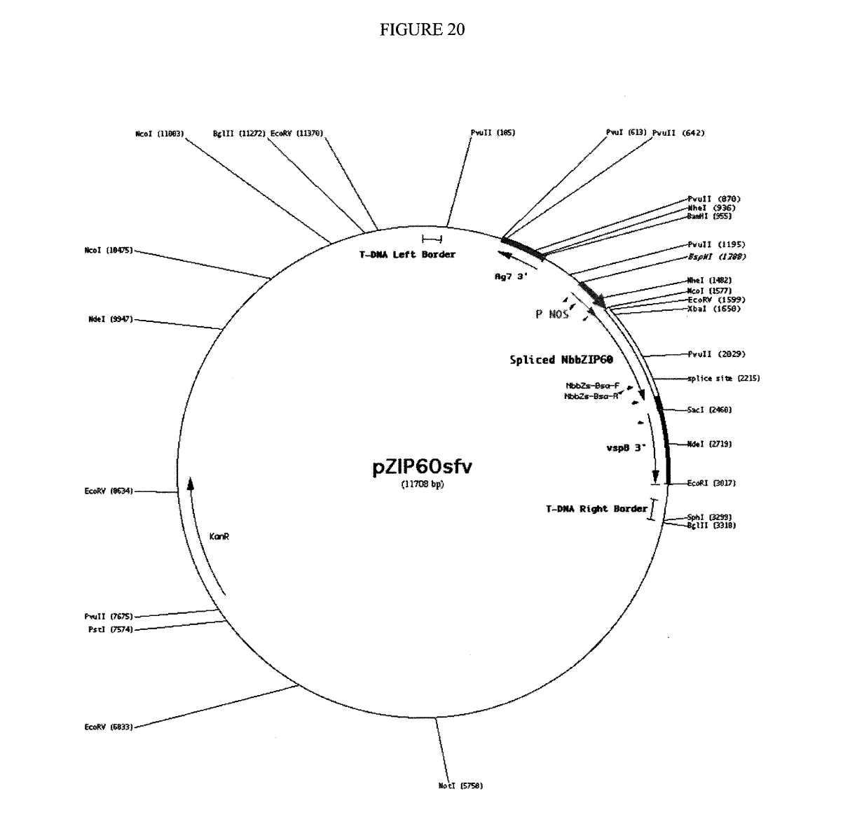

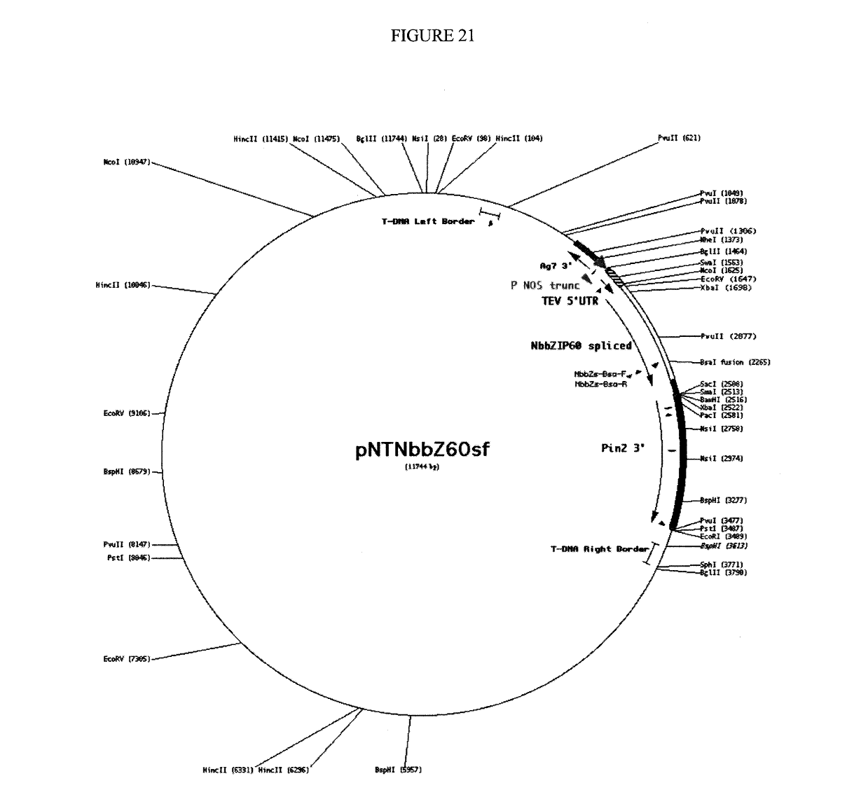

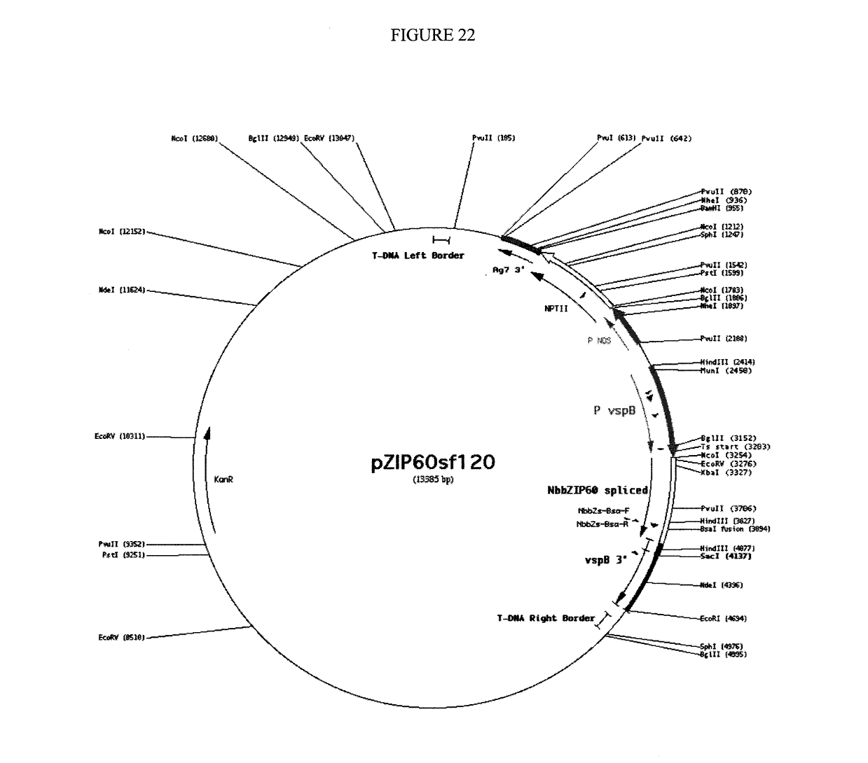

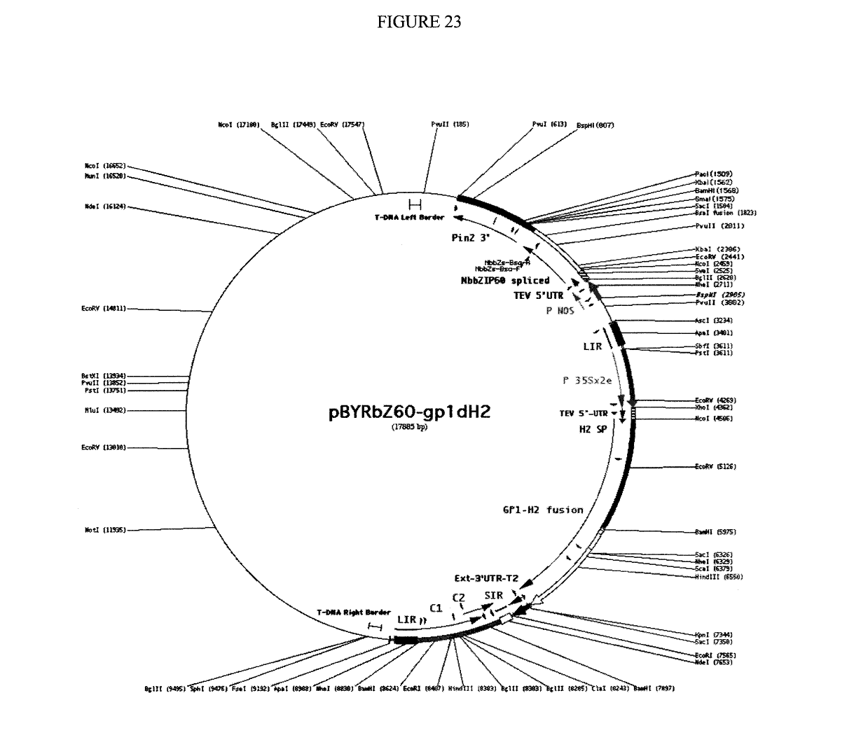

The sequence of the N. benthamiana bZIP60 cDNA was examined and found it to be identical to A. thaliana sequence across the region that was shown to be spliced. Thus, a spliced version (FIG. 19B) of the N. benthamiana bZIP60 (NbbZIP60) cDNA was constructed as follows. The NbbZIP60 cDNA cloned in pICNbbZIP60 was amplified by high-fidelity PCR in two parts. The 5′ segment used the primers IC-F (5′-CACCTCACCCATCTTTTATTAC) and NbbZs-Bsa-R (5′-GGCGGTCTCCAGCAGACTCCTGCTTGGT). The 3′ segment used the primers NbbZs-Bsa-F (5′-GGCGGTCTCCTGCTGTTGGGTTCCCT) and IC-R (5′-TCTCTTCGATTCAAGTGGAG). The 5′ segment was digested with NcoI-BsaI, and the 3′ segment was digested with BsaI-SacI, and the two fragments were ligated with pUC-Np!Kpin2 that was digested with NcoI-SacI. The resulting recombinant clone was verified by DNA sequencing. The spliced NbbZIP60 coding sequence on a NcoI-SacI fragment was ligated into T-DNA expression vectors, including pZIP60sfv (FIG. 20, nopaline synthase (NOS) promoter and soybean vspB terminator), pNTNbbZ60sf (FIG. 21, truncated NOS promoter, tobacco etch virus (TEV) 5′UTR, potato pint terminator), and pZIP60sf120 (FIG. 22, soybean vspB promoter and terminator). Several constructs were produced in order to obtain an expression cassette that provided the optimal level of spliced NbbZIP60 expression. An expression cassette for spliced NbbZIP60 may further be incorporated into a T-DNA vector that contains a geminiviral replicon, e.g. for the expression of Ebola GP1-H2 fusion (FIG. 23).

Transient expression is conducted as described in Example 1. In one instance, one may co-infiltrate N. benthamiana leaves with two Agrobacterium lines, one that carries an T-DNA expression vector for an ER-targeted protein (e.g. Ebola GP1, HCV gpE2) and one that carries a T-DNA expression vector for spliced NbbZIP60 (e.g., FIGS. 20-22). In another instance, one may infiltrate leaves with a single T-DNA vector that contains separate expression cassettes for an ER-targeted protein and for spliced NbbZIP60 (FIG. 23).

Expression may be evaluated by ELISA or Western blotting with antibody probes that are specific for conformation-dependent epitopes and thus can detect correctly folded protein.

### Example 4

### Influenza Virus Hemagglutinin Expression Enhanced by Co-Expression of Calreticulin

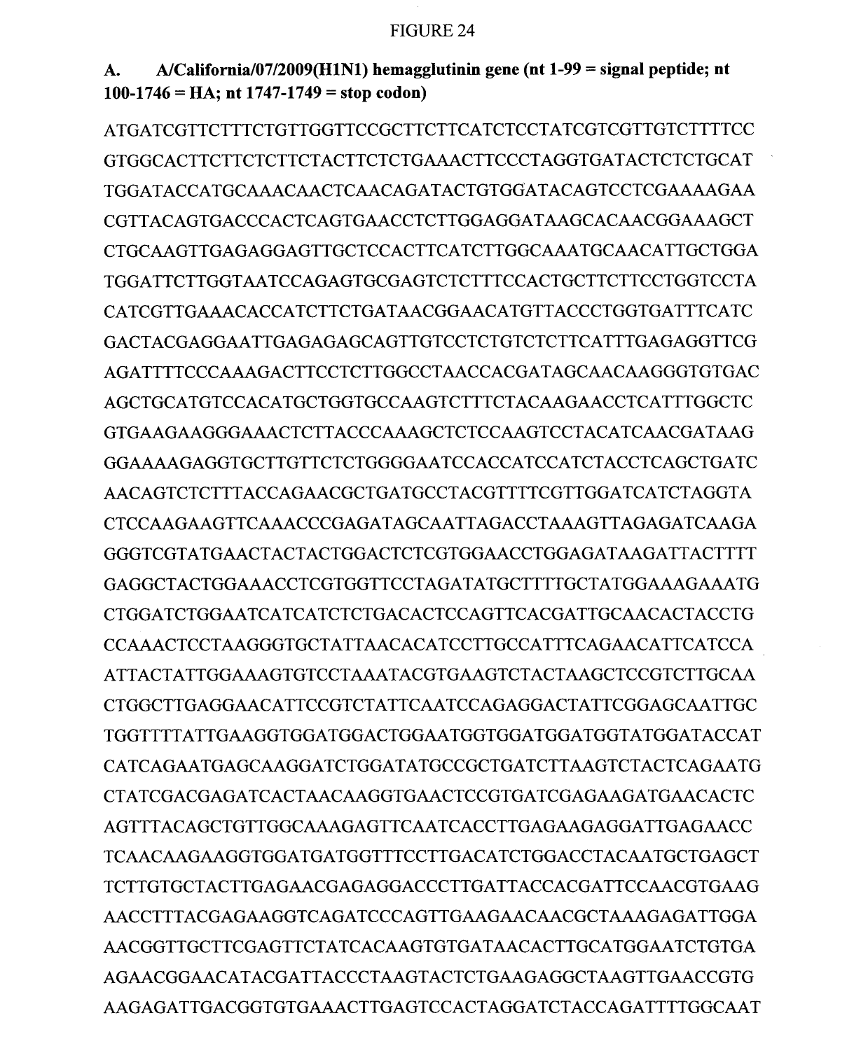

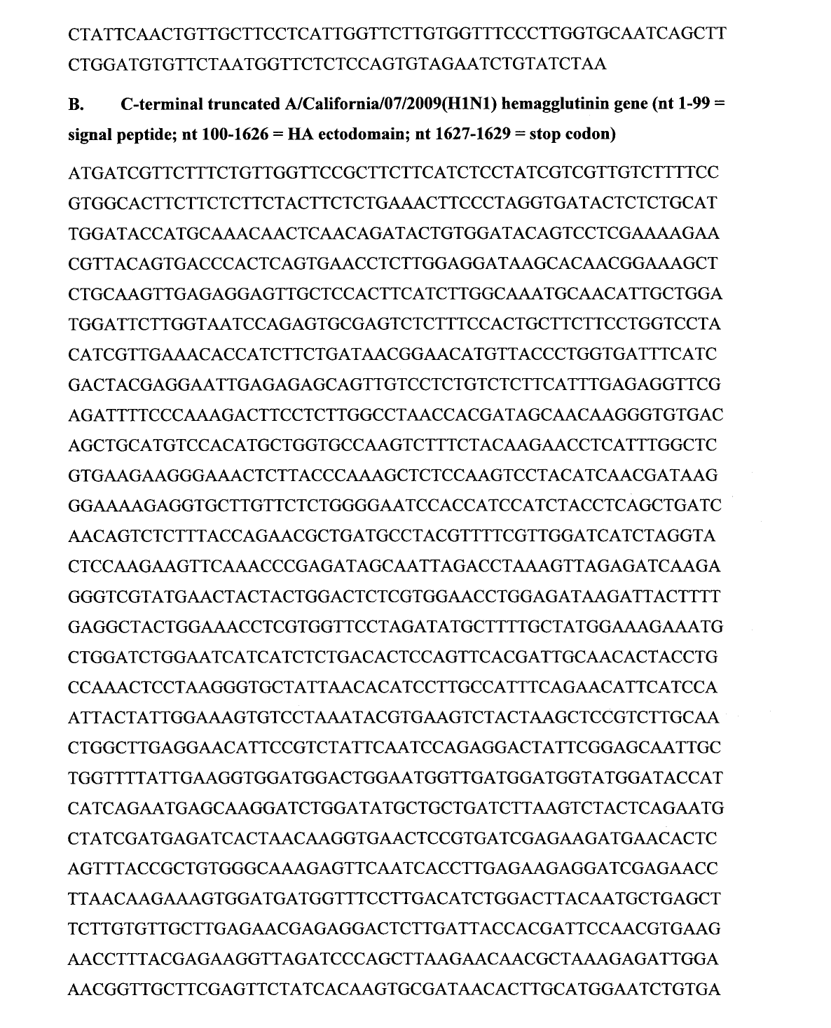

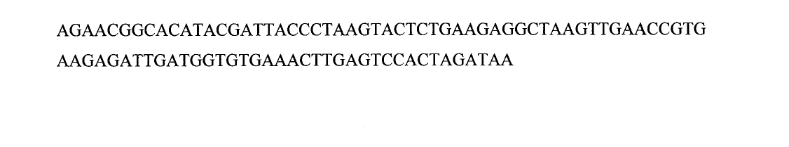

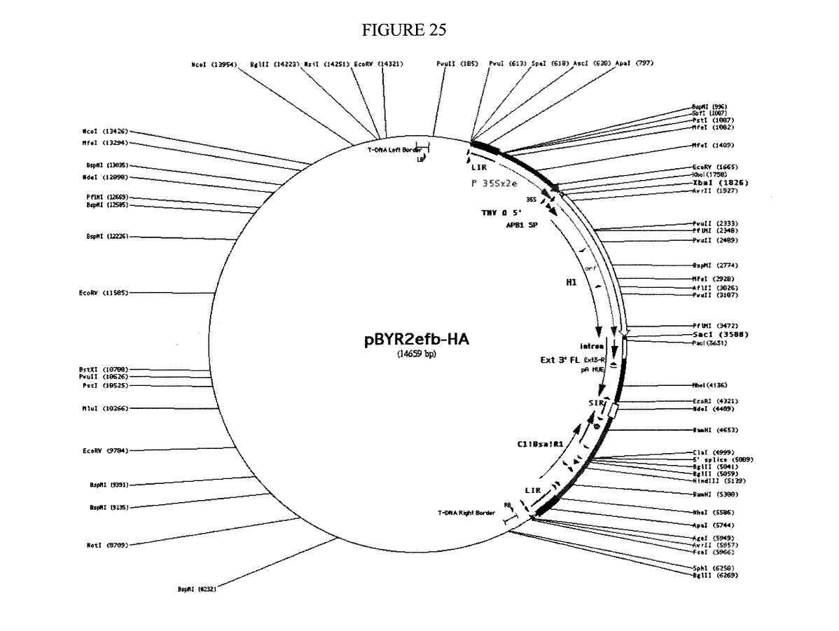

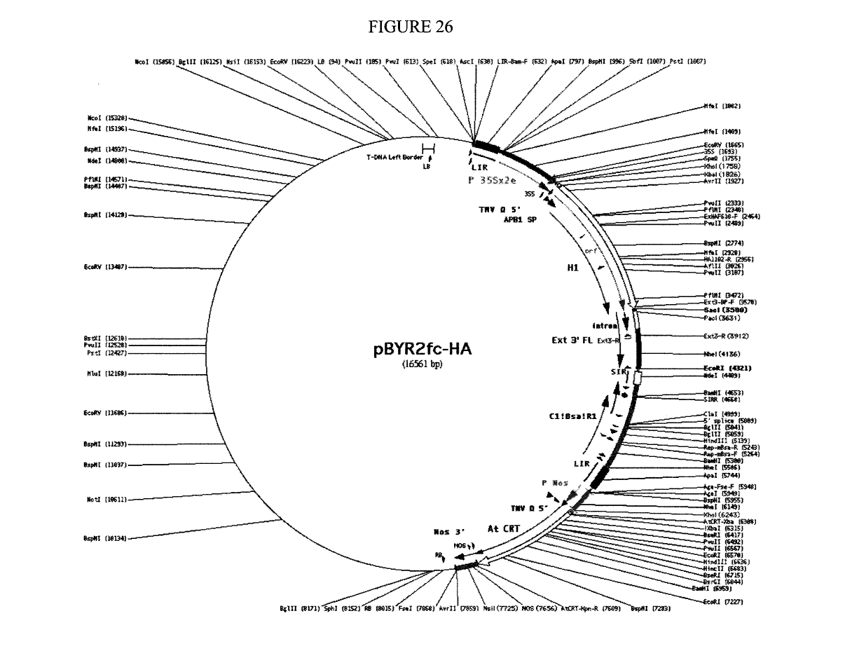

Influenza virus hemagglutinin (HA) is a glycoprotein component of the viral envelope and the major antigenic molecule of the virus and of influenza vaccines. Recombinant HA is an alternative to current virus-based vaccines, and has been produced in plants (D'Aoust, et al., (2008) Plant Biotechnol. J., 6, 930-940; WO 09/076778). Using a geminiviral vector system (Huang et al., (2009) Biotechnol. Bioeng. 103: 706-714; Huang et al., (2010) Biotechnol. Bioeng. 106: 9-17) the expression of a plant-optimized gene encoding HA from virus strain A/California/07/2009 (H1N1) was tested. The gene sequence is provided in FIG. 24A. A C-terminal deletion of the HA gene was also created in order to remove the membrane anchor domain (FIG. 24B). The geminiviral vector pBYR2efb-HA (FIG. 25) containing the full-length HA gene in a geminiviral replicon was constructed. We modified the vector to contain an expression cassette for Arabidopsis thaliana calreticulin (AtCRT), yielding pBYR2fc-HA (FIG. 26). We transferred the T-DNA vectors into Agrobacterium tumefaciens GV3101 and used these clones to inoculate leaves of N. benthamiana as described (Huang et al., (2009) Biotechnol. Bioeng. 103: 706-714). Leaf tissues from triplicate samples were extracted and assayed using a commercial kit ELISA (Sinobiologicals SEK001). The data in Table 2 show that co-expression of AtCRT with HA produced higher levels of HA.

TABLE 2
Expression of HA determined by ELISA in leaf samples
inoculated with pBYR2efb-HA or pBYR2fc-HA.

| Plasmid | Proteins expressed | HA, μg/g leaf mass (mean +/− SD) |
|---|---|---|
| pBYR2efb-HA | HA | 22.6 +/− 4.8 |
| pBYT2fc-HA | HA + CRT | 27.4 +/− 7.8 |

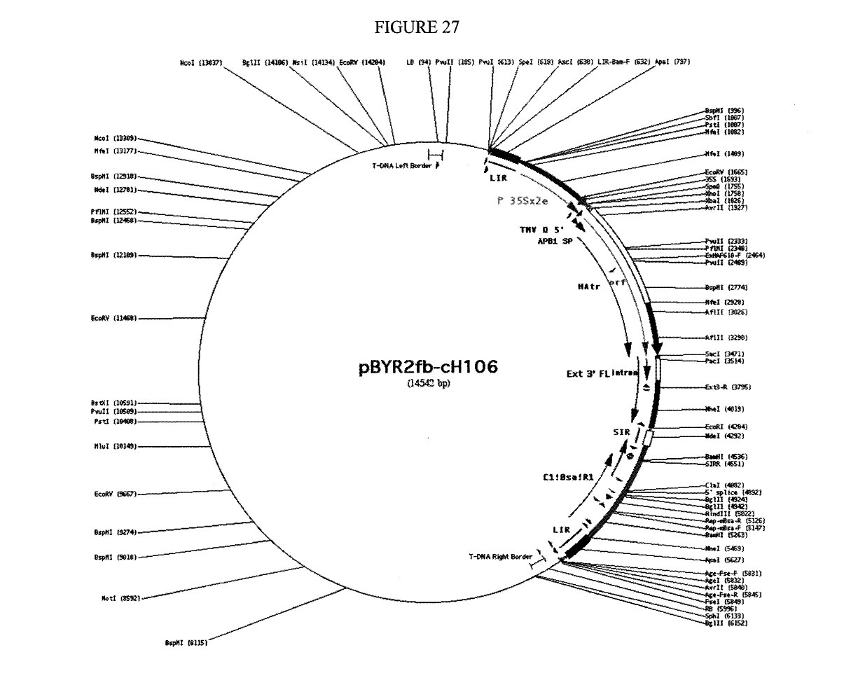

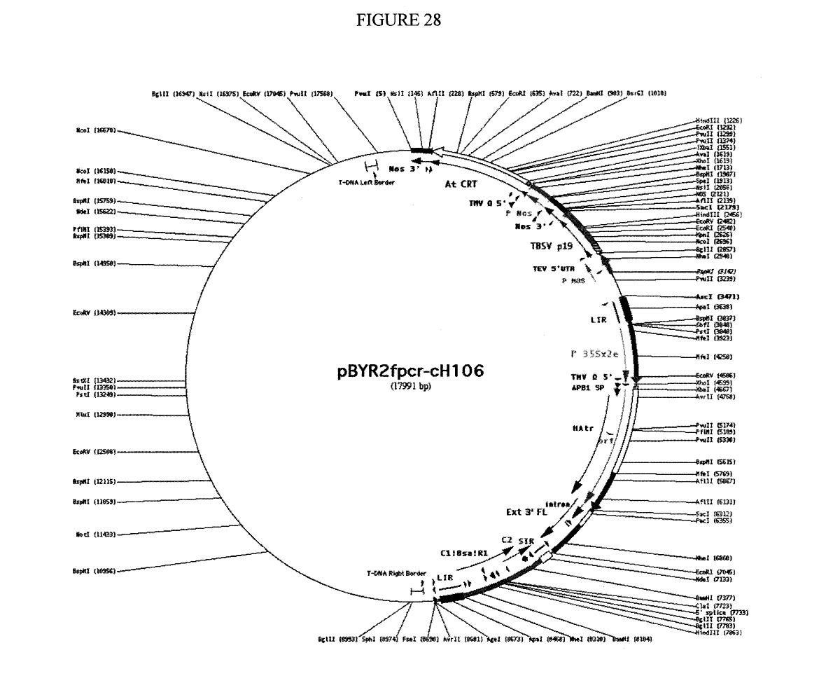

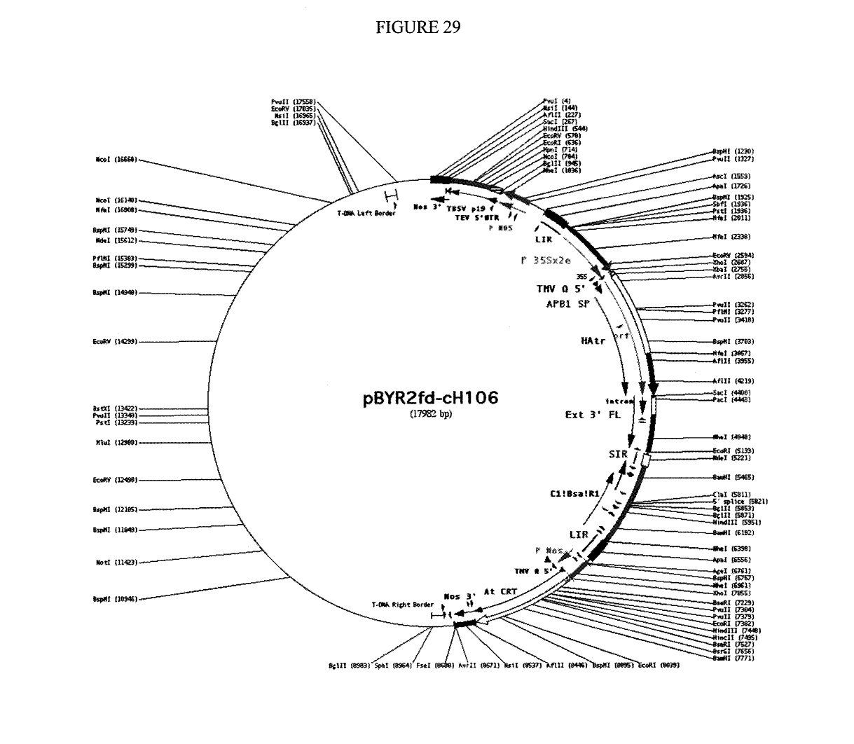

Geminiviral vectors pBYR2fb-cH106, pBYR2fper-cH106, and pBYR2fd-cH106 (FIGS. 27, 28 and 29), containing the C-terminal truncated form of HA gene, were constructed. In addition, pBYR2fper-cH106 contains expression cassettes for AtCRT and the gene silencing inhibitor p19 arranged in tandem adjacent to the left T-DNA border; while pBYR2fd contains the expression cassettes for AtCRT adjacent to the right border and the gene silencing inhibitor p19 adjacent to the left border. We compared expression of the HA ectodomain in benthamiana leaf tissue as described above for full-length HA. The results it Table 3 show that co-expression of AtCRT and p19 greatly enhanced the production of HA, more than doubling in the case of pBYR2fd-cH106. The data also show that the placement of the CRT cassette in relation to the geminiviral replicon containing HA gene, and in relation to the p19 expression cassette, is important. When the AtCRT and p19 genes were placed on either side of the replicon (pBYR2fd-cH106), expression was ˜37% higher than when placed in tandem. This data indicates that AtCRT co-expression results in enhanced production of HA.

TABLE 3
Expression of HA determined by ELISA in leaf samples inoculated
with pBYR2fb-cH016, pBYR2fpcr-cH106, or pBYR2fd-cH106.

| | | HA, μg/g leaf mass |
|---|---|---|
| Plasmid | Proteins expressed | (mean +/− SD) |
| pBYR2fb-cH106 | HA | 50.4 +/− 7.2 |
| pBYR2fpcr-cH106HA | HA + CRT + p19 | 80.5 +/− 28.6 |
| pBYR2fd-cH106 | HA + CRT + p19 | 110.8 +/− 42.6 |

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A plant cell comprising
(i) a first vector comprising two expression cassettes, a first expression cassette comprising a nucleic acid encoding a target protein, wherein the target protein is a recombinant viral glycoprotein, and a second expression cassette comprising a nucleic acid encoding a plant endoplasmic reticulum (ER) protein; or
(ii) a first vector comprising a first expression cassette comprising a nucleic acid encoding a target protein, wherein the target protein is a recombinant viral glycoprotein, and a second vector comprising a second expression cassette comprising a nucleic acid encoding a plant endoplasmic reticulum (ER) protein.

2. The plant cell of claim 1, further comprising a third expression cassette comprising a nucleic acid encoding a plant endoplasmic reticulum protein.

3. The plant cell of claim 1, wherein the nucleic acid in the second expression cassette encodes an ER protein that is different than the ER protein encoded by the nucleic acid in the third expression cassette.

4-9. (canceled)

10. The plant cell of claim 1, wherein the ER protein is an Arabidopsis thaliana, Nicotiana tabacum, Solanum lycopersicum or Nicotiana benthamiana protein.

11. The plant cell of claim 1, wherein the ER protein is a molecular chaperone or a transcription factor.

12. (canceled)

13. The plant cell of claim 11, wherein the ER protein is a molecular chaperone, and the molecular chaperone is selected from a general chaperone, a lectin chaperone and a non-classical chaperone.

14-19. (canceled)

20. The plant cell of claim 11, wherein the ER protein is a transcription factor, the transcription factor is basic leucine zipper transcription factor 60 (bZIP60) or basic leucine zipper transcription factor 28 (bZIP28).

21-24. (canceled)

25. The plant cell of claim 1, wherein the glycoprotein is selected from a HCV protein, an Ebola virus protein and an influenza protein.

26-32. (canceled)

33. The plant cell of claim 1, wherein the first and/or second vector is a geminiviral vector or a non-replicating binary vector.

34. The plant cell of claim 1, wherein the cell is an Arabidopsis thaliana, Nicotiana tabacum, Solanum lycopersicum, or Nicotiana benthamiana cell.

35. A method of making a target protein comprising isolating the target protein from a plant cell of claim 1.

36-43. (canceled)

44. The method of claim 35, wherein an increased amount of properly folded target protein is isolated from the plant cell as compared to an amount of properly folded glycoprotein isolated from a plant cell that was not inoculated with a strain of Agrobacterium comprising a vector comprising an expression cassette comprising a nucleic acid encoding a plant ER protein.

45-51. (canceled)

52. The method of claim 35, wherein the glycoprotein is selected from a HCV protein, an Ebola virus protein and an influenza protein.

53-64. (canceled)

65. The method of claim 35, wherein the first and/or second vector is a geminiviral vector or a non-replicating binary vector.

66. The method of claim 35, wherein the cell is an Arabidopsis thaliana, Nicotiana tabacum, Solanum lycopersicum, or Nicotiana benthamiana cell.

67. A vector comprising two expression cassettes, a first expression cassette comprising a nucleic acid encoding a target protein, wherein the target protein is a recombinant viral glycoprotein, and a second expression cassette comprising a nucleic acid encoding a plant endoplasmic reticulum (ER) protein.

68. A strain of Agrobacterium comprising a vector of claim 67.

69-77. (canceled)

78. A method for improving the recombinant production or yield of protein from a recombinant production in plant cells, which method comprises transiently overexpressing in a host plant cell a molecular chaperone of the endoplasmic reticulum, wherein the plant host cell expresses the recombinant protein.

79-85. (canceled)

86. A method for improving the production of a recombinant HCV E2 protein, which method comprises transfecting a host N. benthamiana cell expressing the recombinant HCV protein with an expression construct wherein expression of said construct in said N. benthamiana that produces a transient expression of at least one or both of the molecular chaperones calnexin, calreticulin and said transient expression of the molecular chaperones leads to an improvement in the production or yield of recombinant HCV E2 protein from said N. benthamiana cell as compared to the production or yield of recombinant HCV E2 protein from said N. benthamiana cell that has not been transfected with such an expression construct.


SEQUENCE LISTING (42 sequences)

SEQ ID NO: 1 - protein, 9 aa, Artificial Sequence
  Description of Artificial Sequence Synthetic peptide
HHHHHHDEL

SEQ ID NO: 2 - DNA, 29 bp, Artificial Sequence
  Description of Artificial Sequence Synthetic primer
agcttctagaacaatggttggaaactggg

SEQ ID NO: 3 - DNA, 26 bp, Artificial Sequence
  Description of Artificial Sequence Synthetic primer
cccgctagcaatacttgatcccacac

SEQ ID NO: 4 - DNA, 40 bp, Artificial Sequence
  Description of Artificial Sequence Synthetic oligonucleotide
ctagccaccatcaccatcaccatgacgagctttaagagct

SEQ ID NO: 5 - DNA, 32 bp, Artificial Sequence
  Description of Artificial Sequence Synthetic oligonucleotide
cttaaagctcgtcatggtgatggtgatggtgg

SEQ ID NO: 6 - DNA, 32 bp, Artificial Sequence
  Description of Artificial Sequence Synthetic primer
cctctagaacaatggcgaaactaaaccctaaa

SEQ ID NO: 7 - DNA, 27 bp, Artificial Sequence
  Description of Artificial Sequence Synthetic primer
ggggtaccttaaagctcgtcatgggcg

SEQ ID NO: 8 - DNA, 34 bp, Artificial Sequence
  Description of Artificial Sequence Synthetic primer
cctctagaacaatgagacaacggcaactattttc

SEQ ID NO: 9 - DNA, 30 bp, Artificial Sequence
  Description of Artificial Sequence Synthetic primer
ggggtaccttgttctaattatcacgtctcg

SEQ ID NO: 10 - DNA, 28 bp, Artificial Sequence
  Description of Artificial Sequence Synthetic primer
tggcatggtgggtgacatcgatgatatc

SEQ ID NO: 11 - DNA, 29 bp, Artificial Sequence
  Description of Artificial Sequence Synthetic primer
ccgagctctcacatcacaattcccaaata

SEQ ID NO: 12 - DNA, 29 bp, Artificial Sequence
  Description of Artificial Sequence Synthetic primer
ccgagctctcaagactcctgcttggtcat

SEQ ID NO: 13 - DNA, 30 bp, Artificial Sequence
  Description of Artificial Sequence Synthetic primer
cgggtacctcaagactcctgcttcgacatc

SEQ ID NO: 14 - DNA, 21 bp, Artificial Sequence
  Description of Artificial Sequence Synthetic primer
gcggtacccgttgtcacgccg

SEQ ID NO: 15 - DNA, 21 bp, Artificial Sequence
  Description of Artificial Sequence Synthetic primer
cgggtacctcaagactcctgc

SEQ ID NO: 16 - DNA, 19 bp, Artificial Sequence
  Description of Artificial Sequence Synthetic primer
gctgctgttcaaggtggta

SEQ ID NO: 17 - DNA, 19 bp, Artificial Sequence
  Description of Artificial Sequence Synthetic primer
tggttgggatgacggtgtt

SEQ ID NO: 18 - DNA, 19 bp, Artificial Sequence
  Description of Artificial Sequence Synthetic primer
gcaacccaattatcacagc

SEQ ID NO: 19 - DNA, 19 bp, Artificial Sequence
  Description of Artificial Sequence Synthetic primer
gtaaccctcacctcaacct

SEQ ID NO: 20 - DNA, 20 bp, Artificial Sequence
  Description of Artificial Sequence Synthetic primer
acggaaaggacatcagcaag

SEQ ID NO: 21 - DNA, 20 bp, Artificial Sequence
  Description of Artificial Sequence Synthetic primer
gtgcccgagtaagtggttca

SEQ ID NO: 22 - DNA, 19 bp, Artificial Sequence
  Description of Artificial Sequence Synthetic primer
gcaacccaattatcacagc

SEQ ID NO: 23 - DNA, 19 bp, Artificial Sequence
  Description of Artificial Sequence Synthetic primer
gtaaccctcacctcaacct

SEQ ID NO: 24 - DNA, 21 bp, Artificial Sequence
  Description of Artificial Sequence Synthetic primer
cctctagaacaatggcgaaac

SEQ ID NO: 25 - DNA, 21 bp, Artificial Sequence
  Description of Artificial Sequence Synthetic primer
ggggtaccttaaagctcgtca

SEQ ID NO: 26 - DNA, 21 bp, Artificial Sequence
  Description of Artificial Sequence Synthetic primer
cctctagaacaatgagacaac

SEQ ID NO: 27 - DNA, 21 bp, Artificial Sequence
  Description of Artificial Sequence Synthetic primer
ggggtaccttgttctaattat

SEQ ID NO: 28 - DNA, 21 bp, Artificial Sequence
  Description of Artificial Sequence Synthetic primer
ctggtggttttgaagctggta

SEQ ID NO: 29 - DNA, 21 bp, Artificial Sequence
  Description of Artificial Sequence Synthetic primer
ggtggtagcatccatcttgtt

SEQ ID NO: 30 - DNA, 22 bp, Artificial Sequence
  Description of Artificial Sequence Synthetic primer
cacctcacccatcttttattac

SEQ ID NO: 31 - DNA, 28 bp, Artificial Sequence
  Description of Artificial Sequence Synthetic primer
ggcggtctccagcagactcctgcttggt

SEQ ID NO: 32 - DNA, 26 bp, Artificial Sequence
  Description of Artificial Sequence Synthetic primer
ggcggtctcctgctgttgggttccct

SEQ ID NO: 33 - DNA, 20 bp, Artificial Sequence
  Description of Artificial Sequence Synthetic primer
tctcttcgattcaagtggag

SEQ ID NO: 34 - DNA, 1152 bp, Artificial Sequence
  Description of Artificial Sequence Synthetic polynucleotide
atggttggaaactgggctaaggtcctggtagtccttttgctctttgctggagtcgacgcagaaactcatgttacaggtggaagtgcaggacacactgtgtctggatttgttagtcttcttgcaccaggagccaaacaaaatgtgcagcttattaacactaatggctcatggcatctcaattcaactgcactgaactgtaatgattctcttaacacaggatggttggcaggtctgttttatcatcacaagttcaattcttcaggatgtcctgaaagattagcctcatgcaggccacttactgattttgatcaaggctggggtcctattagttatgcaaacggatctggacccgaccagagaccatattgttggcactacccaccaaaaccttgcggtattgttcccgctaagtcagtatgtggtcctgtttattgtttcactccatcacccgtggtagttggaacaacagataggagtggcgctccaacatattcctggggtgaaaatgatactgatgtatttgtgcttaacaacactaggccacctttgggaaattggttcggttgtacttggatgaactcaactggattcaccaaagtctgtggtgctcctccttgtgttatcggaggggctggaaacaacaccttgcattgccccactgattgttttagaaaacatcctgatgccacatactctaggtgcggctctggtccttggattacaccaaggtgccttgtcgactacccttataggctttggcattatccttgtactattaactataccatctttaaaattagaatgtatgtgggaggtgtagagcacaggttggaagctgcatgcaattggacaagaggtgaaaggtgcgatttggaagatagggacaggtcagagctttcacctttattgttgacaactacacagtggcaagtgctcccttgttccttcacaaccttaccagccttgtctactggacttatccacctccatcagaacattgttgatgtgcagtatttgtacggtgtgggatcaagtattgcttcctgggccatcaagtgggaatacgttgttttgcttttccttttgcttgctgacgctagagtttgctcatgcttgtggatgatgttattgatatcccaagcagaggcttaa

SEQ ID NO: 35 - DNA, 13953 bp, Artificial Sequence
  Description of Artificial Sequence Synthetic polynucleotide
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgnnnactgagtgcgatattatgtgtaatacataaattgatgatatagctagcttagctcatcgggggatccgtcgaactagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatctggattgagagtgaatatgagactctaattggataccgaggggaatttatggaacgtcagtggagcatttttgacaagaaatatttgctagctgatantgaccttangcgacttttgaacgcgcaataatggnttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgataacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagaatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgagtatttttacaacaattaccaacaacaacaaacaacaaacaacattacaattactatttacaatctagaacaatgatgatggcttctaaggatgctacatcatctgtggatggagctagtggagctggtcaattggttccagaggttaatgcttctgaccctcttgctatggatcctgtagcaggttcttccacagcagttgctactgctggacaagttaatcctattgatccatggataattaacaactttgtgcaagccccccaaggtgaattcactatttccccaaacaacaccccaggtgatgttttgtttgatttgagtttgggtccccatcttaatcctttcttgctccatctctcacaaatgtataatggttgggttggtaacatgagagttaggattatgcttgctggtaatgcctttactgctggtaagataatagtttcttgcataccccctggttttggttcacataatcttactatagcacaagcaactctctttcctcatgtgattgctgatgttaggactcttgaccccattgaggtgcctttggaagatgttaggaatgttctctttcataacaacgatagaaatcaacaaaccatgaggcttgtgtgcatgctctacacccccttgaggactggtggtggtactggtgattcttttgtagttgcaggaagggttatgacttgcccaagtcctgattttaatttcttgtttttagtccctcctacagtggagcaaaaaaccaggcccttcacactcccaaatctcccattgagttctctctctaactcaagagcccctctcccaattagtagtatgggcatttccccagacaatgtccaaagtgtgcaattccaaaatggtaggtgtactcttgatggaagacttgttggcaccaccccagtaagcttgtcacatgttgccaagataagaggtacctccaatggcactgtgatcaaccttactgaattggatggcacaccctttcacccttttgagggccctgcccccattggatttccagatcttggtggttgtgattggcatatcaatatgacacaatttggccattctagccaaacccaatatgatgtcgacaccacccctgacacttttgtcccccatcttggttcaattcaagcaaatggcattggaagtggtaattatgttggtgttctttcttggatttcccccccatcacacccatctggctcccaagttgacctttggaagatccccaattatggatcaagtattactgaggcaacacatcttgccccttctgtatacccccctggttttggagaggtattggtctttttcatgtcaaaaatgccaggtcctggcgcttataatttgccatgtctcttaccacaagagtacatttcacatcttgctagcgagcaagcccctactgtaggtgaggctgccctgctccactatgttgaccctgatactggtaggaatcttggagaattcaaagcataccctgatggtttcctcacttgtgtccccaatggtgctagcagcggtccacaacaactgccaatcaatggtgtctttgtctttgtttcatgggtgtcaagattttatcaattaaagcctgtgggaactgcctctagcgcaagaggtaggcttggtcttaggaggtaagagctcaaagcagaatgctgagctaaaagaaaggctttttccattttcgagagacaatgagaaaagaagaagaagaagaagaagaagaagaagaagaaaagagtaaataataaagccccacaggaggcgaagttcttgtagctccatgttatctaagttattgatattgtttgccctatattttatttctgtcattgtgtatgttttgttcagtttcgatctccttgcaaaatgcagagattatgagatgaataaactaagttatattattatacgtgttaatattctcctcctctctctagctagccttttgttttctctttttcttatttgattttctttaaatcaatccattttaggagagggccagggagtgatccagcaaaacatgaagattagaagaaacttccctcttttttttcctgaaaacaatttaacgtcgagatttatctctttttgtaatggaatcatttctacagttatgacgaattcgagatcggccgcggctgagtggctccttcaatcgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccgga

SEQ ID NO: 36 - DNA, 888 bp, Arabidopsis thaliana
atggcggaggaatttggaagcatagatttactcggagatgaagatttcttcttcgatttcgatccttcaatcgtaattgattctcttccggcggaggattttcttcagtcttcaccggattcatggatcggagaaatcgagaatcaattgatgaacgatgagaatcatcaagaggagagttttgtggaattggatcagcaatcggtttcagatttcatagcggatctactcgttgattatccaactagcgattctggctccgttgatttggcggctgataaagttctaaccgtcgattctcccgccgccgctgatgattccgggaaggagaattcggatttggttgttgagaagaagtctaatgattctggtagcgagattcatgatgatgatgacgaagaaggagacgatgatgctgtggctaaaaaacgaagaaggagagtaagaaatagagatgcggcggttagatcgagagagaggaagaaggaatatgtacaagatttagagaagaagagtaagtatctcgaaagagaatgcttgagactaggacgtatgcttgagtgcttcgttgctgaaaaccagtctctacgttactgtttgcaaaagggtaatggcaataatactaccatgatgtcgaagcaggagtctgctgtgctcttgttggaatccctgctgttgggttccctgctttggcttctgggagtaaacttcatttgcctattcccttatatgtcccacacaaagtgttgcctcctacgtccagaaccagaaaagctggttctaaacgggctcgggagtagtagcaaaccgtcttataccggcgttagtcggagatgtaagggttcgaggcctaggatgaaataccaaatcttaacccttgcggcgtga

SEQ ID NO: 37 - DNA, 651 bp, Artificial Sequence
  Description of Artificial Sequence Synthetic polynucleotide
atggcggaggaatttggaagcatagatttactcggagatgaagatttcttcttcgatttcgatccttcaatcgtaattgattctcttccggcggaggattttcttcagtcttcaccggattcatggatcggagaaatcgagaatcaattgatgaacgatgagaatcatcaagaggagagttttgtggaattggatcagcaatcggtttcagatttcatagcggatctactcgttgattatccaactagcgattctggctccgttgatttggcggctgataaagttctaaccgtcgattctcccgccgccgctgatgattccgggaaggagaattcggatttggttgttgagaagaagtctaatgattctggtagcgagattcatgatgatgatgacgaagaaggagacgatgatgctgtggctaaaaaacgaagaaggagagtaagaaatagagatgcggcggttagatcgagagagaggaagaaggaatatgtacaagatttagagaagaagagtaagtatctcgaaagagaatgcttgagactaggacgtatgcttgagtgcttcgttgctgaaaaccagtctctacgttactgtttgcaaaagggtaatggcaataatactaccatgatgtcgaagcaggagtcttga

SEQ ID NO: 38 - DNA, 900 bp, Nicotiana tabacum
atggtgggtgacatcgatgatatcgttggacacatcaattgggacgatgtagatgacctcttccacaatattctagaagatcacgccgacaatctcttctctgctcatgatccgtccgcgccgtctatccaggagatagagcagcttctcatgaaagacgatgaaatcgtcggtcacgtggctgtcagggagcctgattttcaacttgctgatgactttctctccgacgtgctggccgattctcctgttcagtccgatcattctcactctgataaagtcaatggattccccgattccaaggtttcaagtggctccgaggttgatgatgacgacaaagacaaggagaagggttcccagtcgccgactgagtctaaggacggctccgacgaactaaacagtaacgatcccgtcgataaaaagcgcaagaggcaattgagaaacagggatgcagctgtcaggtcacgagagcggaagaagttgtatgttagggatcttgagttgaagagtagatactttgaatcagagtgcaagaggttggggttagttctccagtgttgtcttgcagaaaatcaagctttgcgtttttctttgcagaatggaagtgctaatggtgcttgtatgaccaagcaggagtctgctgtgctcttgttggaatccctgctgttgggttccctgctttggttcttgggcatcatatgcctgctcattcttcccagccaaccctggttaattccagaagaaaatcaacgaagcagaaaccaccgtcttctggttccaataaagggaggaaataagaatggtcggatttttgagttcgtgtccttcatgatgggcaagagatgcaaagcttcaagatcgaggatgaagttcaatccccattatttgggaattgtgatgtga

SEQ ID NO: 39 - DNA, 900 bp, Nicotiana benthamiana
atggtgggtgacatcgatgatatcgttggacacatcaattgggacgatgtagatcatctcttccacaacattctagaggatcccgccgacaatctcttctctgctcatgatccgtcggcgccgtctatacaggagatcgagcagcttctcatgaacgacgatgatatcgtcggtcacgtggctgtcggagaacctgattttcaacttgctgacgactttctctccgacgtgctagccgattctcctgttcagtccgatcattctccctctgataaagtcattggattctacgattccaaggtttcaagtggctccgaggttgatgatgacgacaaagacaaggagaaggtttcccagtcgccgattgagtctaaggacggctctgacgaactaaacagtgatgatcccgtcgataaaaagcgcaagaggcaattgaggaacagagatgcagctgtcaggtcacgagagaggaagaagttgtatgttagggatcttgagttgaagagtagatactttgaatcagagtgcaagaggttggggttagttctccagtgttgtcttgcagaaaatcaagctttgcgcttctctttgcagagtagcagtgctaatggtgcttgtatgaccaagcaggagtctgctgtgctcttgttggaatccctgctgttgggttccctgctttggttcatgggcatcatatgcctgctcattcttcccagccaaccctggttaattccagaagaaaatcaacgaagcagaaaccacggtcttctggttccaataaagggcggaaataagactggtcggatttttgagttcctgtccttaatgatgggcaagagatgcaaagcttcaagatcgaggatgaagttcaatccccattatttgggaattgtgatgtga

SEQ ID NO: 40 - DNA, 877 bp, Nicotiana benthamiana
atggtgggtgacatcgatgatatcgttggacacatcaattgggacgatgtagatgacctcttccacaatattctagaagatcacgccgacaatctcttctctgctcatgatccgtccgcgccgtctatccaggagatagagcagcttctcatgaaagacgatgaaatcgtcggtcacgtggctgtcagggagcctgattttcaacttgctgatgactttctctccgacgtgctggccgattctcctgttcagtccgatcattctcactctgataaagtcaatggattccccgattccaaggtttcaagtggctccgaggttgatgatgacgacaaagacaaggagaagggttcccagtcgccgactgagtctaaggacggctccgacgaactaaacagtaacgatcccgtcgataaaaagcgcaagaggcaattgagaaacagggatgcagctgtcaggtcacgagagcggaagaagttgtatgttagggatcttgagttgaagagtagatactttgaatcagagtgcaagaggttggggttagttctccagtgttgtcttgcagaaaatcaagctttgcgtttttctttgcagaatggaagtgctaatggtgcttgtatgaccaagcaggagtctgctgttgggttccctgctttggttcttgggcatcatatgcctgctcattcttcccagccaaccctggttaattccagaagaaaatcaacgaagcagaaaccaccgtcttctggttccaataaagggaggaaataagaatggtcggatttttgagttcgtgtccttcatgatgggcaagagatgcaaagcttcaagatcgaggatgaagttcaatccccattatttgggaattgtgatgtga

SEQ ID NO: 41 - DNA, 1749 bp, Influenza A virus
atgatcgttctttctgttggttccgcttcttcatctcctatcgtcgttgtcttttccgtggcacttcttctcttctacttctctgaaacttccctaggtgatactctctgcattggataccatgcaaacaactcaacagatactgtggatacagtcctcgaaaagaacgttacagtgacccactcagtgaacctcttggaggataagcacaacggaaagctctgcaagttgagaggagttgctccacttcatcttggcaaatgcaacattgctggatggattcttggtaatccagagtgcgagtctctttccactgcttcttcctggtcctacatcgttgaaacaccatcttctgataacggaacatgttaccctggtgatttcatcgactacgaggaattgagagagcagttgtcctctgtctcttcatttgagaggttcgagattttcccaaagacttcctcttggcctaaccacgatagcaacaagggtgtgacagctgcatgtccacatgctggtgccaagtctttctacaagaacctcatttggctcgtgaagaagggaaactcttacccaaagctctccaagtcctacatcaacgataagggaaaagaggtgcttgttctctggggaatccaccatccatctacctcagctgatcaacagtctctttaccagaacgctgatgcctacgttttcgttggatcatctaggtactccaagaagttcaaacccgagatagcaattagacctaaagttagagatcaagagggtcgtatgaactactactggactctcgtggaacctggagataagattacttttgaggctactggaaacctcgtggttcctagatatgcttttgctatggaaagaaatgctggatctggaatcatcatctctgacactccagttcacgattgcaacactacctgccaaactcctaagggtgctattaacacatccttgccatttcagaacattcatccaattactattggaaagtgtcctaaatacgtgaagtctactaagctccgtcttgcaactggcttgaggaacattccgtctattcaatccagaggactattcggagcaattgctggttttattgaaggtggatggactggaatggtggatggatggtatggataccatcatcagaatgagcaaggatctggatatgccgctgatcttaagtctactcagaatgctatcgacgagatcactaacaaggtgaactccgtgatcgagaagatgaacactcagtttacagctgttggcaaagagttcaatcaccttgagaagaggattgagaacctcaacaagaaggtggatgatggtttccttgacatctggacctacaatgctgagcttcttgtgctacttgagaacgagaggacccttgattaccacgattccaacgtgaagaacctttacgagaaggtcagatcccagttgaagaacaacgctaaagagattggaaacggttgcttcgagttctatcacaagtgtgataacacttgcatggaatctgtgaagaacggaacatacgattaccctaagtactctgaagaggctaagttgaaccgtgaagagattgacggtgtgaaacttgagtccactaggatctaccagattttggcaatctattcaactgttgcttcctcattggttcttgtggtttcccttggtgcaatcagcttctggatgtgttctaatggttctctccagtgtagaatctgtatctaa

SEQ ID NO: 42 - DNA, 1629 bp, Influenza A virus
atgatcgttctttctgttggttccgcttcttcatctcctatcgtcgttgtcttttccgtggcacttcttctcttctacttctctgaaacttccctaggtgatactctctgcattggataccatgcaaacaactcaacagatactgtggatacagtcctcgaaaagaacgttacagtgacccactcagtgaacctcttggaggataagcacaacggaaagctctgcaagttgagaggagttgctccacttcatcttggcaaatgcaacattgctggatggattcttggtaatccagagtgcgagtctctttccactgcttcttcctggtcctacatcgttgaaacaccatcttctgataacggaacatgttaccctggtgatttcatcgactacgaggaattgagagagcagttgtcctctgtctcttcatttgagaggttcgagattttcccaaagacttcctcttggcctaaccacgatagcaacaagggtgtgacagctgcatgtccacatgctggtgccaagtctttctacaagaacctcatttggctcgtgaagaagggaaactcttacccaaagctctccaagtcctacatcaacgataagggaaaagaggtgcttgttctctggggaatccaccatccatctacctcagctgatcaacagtctctttaccagaacgctgatgcctacgttttcgttggatcatctaggtactccaagaagttcaaacccgagatagcaattagacctaaagttagagatcaagagggtcgtatgaactactactggactctcgtggaacctggagataagattacttttgaggctactggaaacctcgtggttcctagatatgcttttgctatggaaagaaatgctggatctggaatcatcatctctgacactccagttcacgattgcaacactacctgccaaactcctaagggtgctattaacacatccttgccatttcagaacattcatccaattactattggaaagtgtcctaaatacgtgaagtctactaagctccgtcttgcaactggcttgaggaacattccgtctattcaatccagaggactattcggagcaattgctggttttattgaaggtggatggactggaatggttgatggatggtatggataccatcatcagaatgagcaaggatctggatatgctgctgatcttaagtctactcagaatgctatcgatgagatcactaacaaggtgaactccgtgatcgagaagatgaacactcagtttaccgctgtgggcaaagagttcaatcaccttgagaagaggatcgagaaccttaacaagaaagtggatgatggtttccttgacatctggacttacaatgctgagcttcttgtgttgcttgagaacgagaggactcttgattaccacgattccaacgtgaagaacctttacgagaaggttagatcccagcttaagaacaacgctaaagagattggaaacggttgcttcgagttctatcacaagtgcgataacacttgcatggaatctgtgaagaacggcacatacgattaccctaagtactctgaagaggctaagttgaaccgtgaagagattgatggtgtgaaacttgagtccactagataa